(19) United States
(12) Patent Application Publication
(10) Pub. No.: US 2006/0223777 A1
(43) Pub. Date: Oct. 5, 2006

(54) Highly functional short hairpin RNA

(75) Inventors: Annaleen Vermeulen, Lafayette, CO (US); Angela Reynolds, Conifer, CO (US); Jon Karpilow, Boulder, CO (US); Devin Leake, Denver, CO (US); Xiaoqin Cheng, Broomfield, CO (US); Stephanie A. Hartsel, Berthoud, CO (US); Anastasia Khvorova, Boulder, CO (US)

Correspondence Address: WORKMAN NYDEGGER;(F/K/A WORKMAN NYDEGGER & SEELEY), 60 EAST SOUTH TEMPLE, 1000 EAGLE GATE TOWER, SALT LAKE CITY, UT, 84111, US

(73) Assignee: Dharmacon, Inc.

(21) Appl. No.: 11/390,829

(22) Filed: Mar. 28, 2006

Related U.S. Application Data
(60) Provisional application No. 60/666,474, filed on Mar. 29, 2005.

(51) Int. Cl. A61K 48/00; C07F 9/6512; C07H 21/02; C12N 15/00

(52) U.S. Cl. 514/44; 536/23.1; 514/81; 435/455; 544/243

(57) ABSTRACT

The present invention provides improved hairpin and fractured hairpin constructs for use in gene silencing through the RNA interference pathway. An exemplary short hairpin polynucleotide for use in gene silencing can include a polynucleotide having from about 42 nucleotides to about 106 nucleotides configured for being processed by Dicer. The polynucleotide can include a first region having from about 19 to about 35 nucleotides, a loop region coupled to the first region, the loop region having from about 4 to about 30 nucleotides, and a second region having from about 19 to about 35 nucleotides and having at least about 80% complementarity to the first region. Optionally, one of the first region or second region can have an overhang having less than about 6 nucleotides. Also, the short hairpin can be formed of a plurality of polynucleotides that cooperate to form a hairpin structure.

### CROSS-REFERENCE TO RELATED APPLICATIONS

This patent application claims benefit of U.S. Provisional Application Ser. No. 60/666,474, entitled “HIGHLY FUNCTIONAL SHRNA,” filed Mar. 29, 2005, which provisional application is incorporated herein by reference.

### BACKGROUND OF THE INVENTION

1. The Field of the Invention

The present invention relates to unimolecular RNA that can function within the RNA interference pathway. More particularly, the present invention relates to unimolecular RNA that form short hairpin RNA (“shRNA”) that can function in the RNA interference pathway to induce gene silencing.

2. The Related Technology

RNA interference (“RNAi”) is a cellular process in eukaryotic cells in which the enzymatic degradation of mRNA is directed by double stranded RNA (“dsRNA”) that share substantial homology with a target mRNA. This phenomenon was first observed in plants in 1990 (Napoli, C., C. Lemieux, and R. Jorgensen, Introduction of a chimeric chalcone synthase gene into petunia results in reversible co-suppression of homologous genes in trans. Plant Cell, 1990. 2(4): p. 279-289) and later identified in other eukaryotes including fungi, worms, and other organisms (Romano, N. and G. Macino, Quelling: transient inactivation of gene expression in Neurospora crassa by transformation with homologous sequences. Mol. Microbiol., 1992. 6(22): p. 3343-53). Mechanistically, it is now known that long dsRNA can be cleaved into short interfering RNA (“siRNAs”) duplexes by Dicer, a Type III RNase. Subsequently, these small duplexes interact with the RNA Induced Silencing Complex (“RISC”), a multisubunit complex that contains both helicases and endonuclease activities that mediate degradation of homologous transcripts. While initial attempts to induce RNAi in mammalian cells were unsuccessful, which may be due to the interferon response pathway, it was later discovered that mammalian cells transfected with synthetic siRNAs could induce the RNAi pathway (Elbashir, S. M., et al., Duplexes of 21-nucleotide RNAs mediate RNA interference in cultured mammalian cells. Nature, 2001. 411(6836): p. 494-8).

While RNAi has vast potential in gene function analysis, drug discovery programs, therapeutics and the like, a number of challenges have arisen over the course of its development. First, researchers have observed wide-ranging variability in the level of silencing induced by different siRNA. Also, siRNA that are selected at random may induce 75% knockdown of the intended target at a moderate frequency (e.g., 50-55%), making these reagents less than dependable. However, recent development of more advanced selection methods, including algorithm-based rational design selection, can select siRNA by key sequence and thermodynamic parameters that are target sequence independent. Rationally designed siRNA have provided the ability to choose systematically more potent duplexes (Khvorova, A., Reynolds, A. and Jayasena, S. Functional siRNAs and miRNAs exhibit strand bias. Cell, 2003. 115(1): p. 209-216; U.S. patent application Ser. No. 10/714,333, filed Nov. 13, 2003; U.S. patent application Ser. No. 10/940,892, filed Sep. 14, 2004, which are incorporated herein by reference).

Identification of important sequence and thermodynamic parameters has been based on data obtained from testing of siRNA comprised of two separate strands. However, molecules of siRNA comprised of two separate strands may have disadvantages when used as therapeutic compounds because of the possibility of either separate strand being present alone as an impurity. Current regulations require that for FDA approval of bipartite molecules, such as siRNA comprised of two separate strands, both the duplex and the individual strands that make up the duplex must be thoroughly tested. As these essential and necessary procedures dramatically amplify the cost of drug development, alternatives, such as unimolecular structures, are highly desirable.

Therefore, it would be advantageous to have a reliable and convenient method of converting siRNA comprised of two separate strands into highly functional unimolecular siRNA that can be chemically synthesized or expressed from a vector. Also, it would be beneficial to have a unimolecular siRNA design that provides improved silencing regardless of the functionality of the related siRNA formed from two separate strands.

### SUMMARY OF THE INVENTION

The present invention is directed to methods and compositions pertaining to hairpin and fractured hairpin nucleic acids for use in gene silencing. Accordingly, the present invention provides kits, compositions, and methods for increasing the efficiency of RNA interference.

In one embodiment, the present invention can include a polynucleotide for use in gene silencing. Such a polynucleotide can be RNA and/or DNA, and include from about 42 nucleotides to about 106 nucleotides configured for being processed by Dicer. Additionally, the polynucleotide can include the following: a first region having from about 19 to about 35 nucleotides; a loop region coupled to the first region, the loop region having from about 4 to about 30 nucleotides; a second region having from about 19 to about 35 nucleotides and having at least about 80% complementarity to the first region; and optionally, an overhang region on one of the first region or second region and having less than about 6 nucleotides.

Additionally, the polynucleotide can include about 71 nucleotides. The polynucleotide can also include at least one of the following: the first region having about 31 nucleotides; the loop region having about 7 nucleotides; the second region having about 31 nucleotides; or an overhang region having 2 nucleotides. Preferably, the loop region comprises nucleotides having the sequence of 5′-AUAUGUG-3′ (SEQ. ID. NO. 1).

The polynucleotide can also have at least one of the following: a sense region having a first 5′ sense nucleotide and a second 5′ sense nucleotide, wherein the first and second 5′ sense nucleotides have a 2′ modification; an antisense region having no antisense nucleotides with a 2′ modification; an antisense region having a second 5′ antisense nucleotide with a 2′ modification; or an antisense region having a first 5′ antisense with no 2′ modification. Usually, the 2′ modification is a 2′-O-alkyl modification, and preferably a 2′-O-methyl modification.

The polynucleotide can be configured to be processed by Dicer in order to form a duplex having a sense strand and an antisense strand. The duplex produced by Dicer can include at least one of the following: a sense strand having a first 5′ sense nucleotide at a first terminal nucleotide position and a second 5′ sense nucleotide at a second nucleotide position adjacent to the terminal nucleotide position, wherein the first and second 5′ sense nucleotides have a 2′-O-alkyl modification; an antisense strand having no antisense nucleotides with a 2′ modification; an antisense strand having a first 5′ antisense nucleotide at a first terminal nucleotide position and a second 5′ antisense nucleotide at a second nucleotide position adjacent to the terminal nucleotide position, wherein the second 5′ antisense nucleotide includes a 2′-O-alkyl modification; or an antisense region having a first 5′ antisense nucleotide at a first terminal position with no 2′ modification. Also, the first 5′ antisense nucleotide includes a 5′ phosphate group.

In one embodiment, the present invention can include a plurality of polynucleotides that are capable of forming a fractured hairpin for use in gene silencing. A fractured hairpin can include a first polynucleotide strand, and a second polynucleotide strand. The second polynucleotide can be capable of forming a hairpin structure with the first polynucleotide that can be processed by Dicer. Usually, the hairpin structure can have from about 42 to about 106 nucleotides. Accordingly, the second polynucleotide strand can include the following: a first region having at least 80% complementarity with the first strand and can be capable of forming a first duplex region with the first strand; a second region coupled to the first region; a third region coupled to the second region; and a fourth region coupled to the third region and having at least 80% complementarity with the second region, wherein the fourth region can be capable of forming a second duplex region with the second region such that the third region forms a loop adjacent to the second duplex region.

Optionally, the fractured hairpin can include an overhang region having less than about 6 nucleotides and preferably 2 nucleotides on one of the first polynucleotide strand or first region of the second polynucleotide strand. Also, the third region that forms a loop can include nucleotides having the sequence of 5′-AUAUGUG-3′ (SEQ. ID. NO. 1). Further, the first strand can include an antisense nucleotide with a 5′ phosphate group. In one option, the first strand antisense nucleotide with the 5′ phosphate group is adjacent to the fourth region of the second strand. In another option, the first strand antisense nucleotide with the 5′ phosphate group is not adjacent to the fourth region of the second strand.

The fractured hairpin can be processed by Dicer such that the first polynucleotide strand and second polynucleotide strand can result in at least one of the following: a sense strand having a first 5′ sense nucleotide at a first terminal nucleotide position and a second 5′ sense nucleotide at a second nucleotide position adjacent to the terminal nucleotide position, wherein the first and second 5′ sense nucleotides have a 2′-O-alkyl modification; an antisense strand having no antisense nucleotides with a 2′ modification; an antisense strand having a first 5′ antisense nucleotide at a first terminal nucleotide position and a second 5′ antisense nucleotide at a second nucleotide position adjacent to the terminal nucleotide position, wherein the second 5′ antisense nucleotide includes a 2′-O-alkyl modification; or an antisense region having a first 5′ antisense nucleotide at a first terminal position with no 2′ modification.

In one embodiment, the present invention can include a short hairpin RNA for use in gene silencing. The short hairpin RNA can include at least one polynucleotide, and have from about 42 nucleotides to about 106 nucleotides. Also, the short hairpin can be configured for being processed by Dicer. Additionally, the short hairpin RNA can include the following: a first region; a loop region coupled to the first region; and a second region coupled to the loop region and being capable of forming a first duplex region with the first region. The short hairpin RNA can be characterized by at least one of the first or second region having at least two tandem nucleotides. Each of the two tandem nucleotides can include a 2′ modification such that processing by Dicer results in a sense strand having a first 5′ sense nucleotide at a first terminal nucleotide position and a second 5′ sense nucleotide at a second nucleotide position adjacent to the terminal nucleotide position, wherein each of the first and second 5′ sense nucleotides have the 2′-modification. Preferably, the 2′ modification is a 2′-O-alkyl modification, and more preferably it is a 2′-O-methyl modification.

Optionally, the first or second region can include a nucleotide having a 2′ modification such that processing by Dicer results in an antisense strand having a first 5′ sense nucleotide at a first terminal nucleotide position and a second 5′ sense nucleotide at a second nucleotide position adjacent to the terminal nucleotide position, wherein the second 5′ sense nucleotide has the 2′-modification. Alternatively, the processing by Dicer can result in an antisense strand substantially devoid of nucleotides having a 2′ modification.

Also, the short hairpin RNA can have about 71 nucleotides. Such a short hairpin RNA can be comprised of at least one of the following: the first region having about 31 nucleotides; the loop region having about 7 nucleotides; the second region having about 31 nucleotides; or an overhang region having 2 nucleotides. Also, the loop region can have the sequence of SEQ. ID. NO. 1.

In one embodiment, the present invention provides a nucleic acid comprising a single strand of RNA with a first region, a loop region, and a second region. Preferably the first region and the second region are substantially complementary to each other and are each between 19 and 35 nucleotides in length, more preferably between 26 and 32 nucleotides in length. The first region or the second region will preferably contain at least 19 nucleotides that are at least substantially complementary to the target mRNA. Under one particularly preferred embodiment, the first region and the second region each comprise 31 nucleotides, the loop region is 7 bases long and there is an overhang region of 2 bases on either the 5′ or the 3′ end of the molecule, thereby forming an unimolecular ribonucleic acid of 71 bases. In addition, preferably the loop structure is 5′-AUAUGUG-3′, (SEQ. ID NO. 1) derived from the hsa-mir-17 sequence.

In another embodiment, the present invention provides a fractured hairpin molecule. A fractured hairpin molecule is comprised of at least two separate strands: a first strand; and a second strand. In the case where the fractured hairpin comprises two different strands, the second strand comprises four different regions (I, II, III, IV) where the first region (I) is capable of annealing or hybridizing with the first strand, the second region (II) is capable of annealing or hybridizing with the fourth region (IV), and the third region (III), which is physically located between the second region (II) and the fourth region (IV), forms a loop structure.

Similarly, under one preferred embodiment, the nucleotide composition of the fractured hairpins is 71 bases, where the complete sense and antisense regions each comprise 31 bases, there is a two base overhang on either the 5′ or the 3′ end of the fractured hairpin molecule and the loop structure is 7 bases long.

Additionally, the present invention can provide hairpin and/or fractured hairpin molecules that after Dicer processing yield duplexes that are comprised of two separate strands that have desired modifications that enhance stability and/or specificity of the resulting duplex.

Moreover, present invention can be directed to a method for inducing gene silencing with the polynucleotides of the present invtention. The method comprises exposing a hairpin or fractured hairpin to a cell that is expressing or is capable of expressing said target nucleic acid.

These and other advantages and features of the present invention will become more fully apparent from the following description and appended claims, or may be learned by the practice of the invention as set forth hereinafter.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings:

FIGS. 1A-1F depict the structure of embodiments of hairpins containing various modification patterns and the predicted molecules that are derived from these structures upon Dicer processing.

FIG. 2 depicts an outline of an embodiment of an ACE synthesis cycle.

FIG. 3 depicts the structure of an embodiment of a 2′ ACE protected RNA.

FIGS. 4A-4D depict the structures of various embodiments of fractured hairpins and the predicted products of these molecules upon Dicer digestion. In cases where the fractured hairpin has the structure shown in FIG. 4A, preferably, the 5′ end of the antisense strand is phosphorylated and the 5′ end of the sense strand is either unphosphorylated or contains one or more modifications that block phosphorylation. In cases where the fractured hairpin has the structure shown in FIG. 4B, preferably the 5′ end of the sense strand is either unphosphorylated or contains one or more modifications that block phosphorylation. In cases where the fractured hairpin has the structure shown in FIG. 4C, preferably, the 5′ end of the antisense strand is phosphorylated. In cases where the fractured hairpin has the structure shown in FIG. 4D, preferably, the 5′ end of the antisense strand is phosphorylated and the 5′ end of the sense strand is unphosphorylated or contains one or more modifications that block phosphorylation.

FIGS. 5A-5B depict an embodiment of a synthesis protocol for fractured hairpins.

FIG. 6 depicts an embodiment of a synthesis protocol for fractured hairpins using donor-acceptor groups. Using strands that are modified with acceptor and donor groups can yield a modified hairpin.

FIG. 7 depicts the performance of embodiments of siRNA and shRNA (e.g., both right-handed and left-handed loops) targeting the DBI gene.

FIG. 8 depicts the performance of embodiments of siRNA and shRNA (e.g., both right-handed and left-handed loops) targeting human cyclophilin B and SEAP.

FIGS. 9A-9B depict the performance of an embodiment of shRNA, siRNA, and 31 mer siRNA directed against DBI.

FIGS. 10A-10D depict the functionality of an embodiment of shRNA having stem lengths ranging from 17-31 base pairs at four different concentrations (e.g., 100, 10, 1, and 0.1 nM).

FIGS. 11A-11B show the structure of embodiments of hairpins used in studies that compare the functionality of shRNA and fractured shRNA targeting DBI. The siRNA targeted by both of these sequences (e.g., DBI25 and DBI34) provide less than 50% silencing.

FIGS. 11C-11D show the level of silencing obtained with shRNA and fractured hairpins using different organizations (e.g., AS-//-AS-loop-S).

FIG. 12 is a polyacrylamide gel comparing the pattern of fragments obtained when Dicer digests dsRNA and fractured shRNA. The “*” represents a labeled phosphate.

### DETAILED DESCRIPTION OF THE PREFERRED EMBODIMENTS

The present invention will now be described in connection with preferred embodiments. These embodiments are presented to aid in an understanding of the present invention and are not intended, and should not be construed, to limit the invention in any way. All alternatives, modifications, and equivalents that may become apparent to those of ordinary skill upon reading this disclosure are included within the spirit and scope of the present invention.

As such, this disclosure is not a primer on compositions and methods for performing RNA interference or shRNA. Basic concepts known to those skilled in the art have not been set forth in detail.

Embodiments of the present invention can be directed to compositions and methods for performing RNA interference, including shRNA-induced gene silencing. Through the use of shRNA, modified shRNA, fractured shRNA, modified fractured shRNA, and derivatives thereof, the efficiency of RNA interference may be improved. Accordingly, the present invention provides kits, compositions, and methods for increasing the efficiency of RNA interference.

Generally, shRNA can include a unimolecular RNA having a first region, a loop region, and a second region. Preferably the first region and the second region are substantially complementary to each other, and each can be between 19 and 35 nucleotides in length, and more preferably between 26 and 32 nucleotides in length. The first region and/or the second region can contain at least nineteen bases that are complementary to a target mRNA. In a particularly preferred embodiment, the first region and the second region can each comprise 31 nucleotides, the loop region is 7 bases long and there is an overhang region of 2 bases on either the 5′ or the 3′ end of the molecule, thereby forming an unimolecular RNA of 71 bases. There also may be an overhang that is located on the 5′ or 3′ end of either the first and/or the second strand, which can be comprised of one to six bases. Preferably, the overhang is present on the 3′ end of one or both strands. In addition, preferably the loop structure is comprised of a polynucleotide having a 5′-AUAUGUG-3′, (SEQ. ID NO. 1) sequence derived from the hsa-mir-17 sequence.

Additionally, the present invention can include a fractured hairpin siRNA (“sfhRNA”). An sfhRNA is comprised of at least two separate strands: a first strand; and a second strand. In the case where the sfhRNA comprises two different strands, the second strand comprises four different regions (I, II, III, IV) as follows: the first region (I) is capable of annealing or hybridizing with the first strand; the second region (II) is capable of annealing or hybridizing with the fourth region (IV); and the third region (III), which is physically located between the second region (II); and the fourth region (IV) forms a loop structure. Similarly, when the sfhRNA is 71 bases, the complete sense and antisense regions can each comprise 31 bases, and there is a two base overhang on either the 5′ or the 3′ end of the sfhRNA and the loop structure is 7 bases long. The total length of the fractured hairpin is such that the first strand and second strand are capable of forming a fractured hairpin that contains a region of substantially, if not 100% self-complementary between 19 and 35 nucleotides. Further, as with the first embodiment, preferably the loop structure is 5′-AUAUGUG-3′ (SEQ. ID NO. 1) derived from the hsa-mir-17 sequence.

The shRNA and/or sfhRNA can undergo Dicer processing that yields duplexes that are comprised of two separate strands that have desired modifications that enhance stability and/or specificity of the resulting duplex. The modifications and their locations within molecules are described in a number of commonly owned applications including PCT/US04/10343, which was filed on Apr. 1, 2004 and published as WO 2004/090,105 A2 on Oct. 21, 2004; PCT/US 04/14270, which was filed on May 6, 2004 and published as WO 2004/099,387 A2 on Nov. 18, 2004, PCT/US2005/003,365 which was filed on Feb. 4, 2005; and U.S. patent application Ser. No. 11/019,831, which was filed on Dec. 22, 2004, each of which is incorporated by reference herein.

Moreover, the shRNA and/or sfhRNA having the design of the invention can be useful in implementing gene silencing. Also, they may be preferred over duplexes having lengths that are similar or equivalent to the length of the stem of the hairpin in some instances, due to the fact that shRNA and/or sfhRNAs of this design can be less likely to induce cellular stress and/or toxicity.

A. Definitions

The following terminology is defined herein to clarify the terms used, in describing embodiments of the present invention and is not intended to be limiting. As such, the following terminology is provided to supplement the understanding of such terms by one of ordinary skill in the relevant art.

As used herein, the term “antisense strand” is meant to refer to a polynucleotide or region of a polynucleotide that is at least substantially (e.g., about 80% or more) or 100% complementary to a target nucleic acid of interest. Also, the antisense strand of a dsRNA is at least substantially complementary to its sense strand. An antisense strand may be comprised of a polynucleotide region that is RNA, DNA, or chimeric RNA/DNA. Additionally, any nucleotide within an antisense strand can be modified by including substituents coupled thereto, such as in a 2′ modification. The antisense strand can be modified with a diverse group of small molecules and/or conjugates. For example, an antisense strand may be complementary, in whole or in part, to a molecule of messenger RNA (“mRNA”), an RNA sequence that is not mRNA including non-coding RNA (e.g., tRNA and rRNA), or a sequence of DNA that is either coding or non-coding. The terms “antisense strand” and “antisense region” are intended to be equivalent and are used interchangeably.

The antisense region or antisense strand may be part of a larger strand that comprises nucleotides other than antisense nucleotides. For example, in the case of a unimolecular structure the larger strand would contain an antisense region, a sense region and a loop region, and might also contain overhang nucleotides and additional stem nucleotides that are complementary to other stem nucleotides, but not complementary to the target. In the case of a fractured hairpin, the antisense region may be part of a strand that also comprises overhang nucleotides and/or a loop region and two other regions that are self-complementary.

As used herein, the term “2′ carbon modification” refers to a nucleotide unit having a sugar moiety, for example a moiety that is modified at the 2′ position of the sugar subunit. A “2′-O-alkyl modified nucleotide” is modified at this position such that an oxygen atom is attached both to the carbon atom located at the 2′ position of the sugar and to an alkyl group. Examples include 2′-O-methyl, 2′-O-ethyl, 2′-O-propyl, 2′-O-isopropyl, 2′-O-butyl, 2-O-isobutyl, 2′-O-ethyl-O-methyl (—OCH₂CH₂OCH₃), 2′-O-ethyl-OH (—OCH₂CH₂OH) and the like. A “2′ carbon sense modification” refers to a modification at the 2′ carbon position of a nucleotide on the sense strand or within a sense region of polynucleotide. A “2′ carbon antisense modification” refers to a modification at the 2′ carbon position of a nucleotide on the antisense strand or within an antisense region of polynucleotide.

As used herein, the terms “complementary” and “complementarity” are meant to refer to the ability of polynucleotides to form base pairs with one another. Base pairs are typically formed by hydrogen bonds between nucleotide units in anti-parallel polynucleotide strands. Complementary polynucleotide strands can base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine.

Perfect complementarity or 100% complementarity refers to the situation in which each nucleotide unit of one polynucleotide strand can hydrogen bond with a nucleotide unit of an anti-parallel polynucleotide strand. Less than perfect complementarity refers to the situation in which some, but not all, nucleotide units of two strands can hydrogen bond with each other. For example, for two 20-mers, if only two base pairs on each strand can hydrogen bond with each other, the polynucleotide strands exhibit 10% complementarity. In the same example, if 18 base pairs on each strand can hydrogen bond with each other, the polynucleotide strands exhibit 90% complementarity. “Substantial complementarity” refers to polynucleotide strands exhibiting 79% or greater complementarity, excluding regions of the polynucleotide strands, such as overhangs, that are selected so as to be non-complementary. Accordingly, complementarity does not consider overhangs that are selected so as not to be similar or complementary to the nucleotides on the anti-parallel strand.

As used herein, the terms “fractured hairpin” or “sfhRNA” refers to a hairpin that comprises two or more distinct strands. Such molecules can be organized in a variety of fashions (e.g., 5′-sense-fracture-sense-loop-antisense, 5′-sense-loop-antisense-fracture-antisense, 5′ antisense-fracture-antisense-loop-sense, 5′ antisense-loop-sense-fracture-sense) and the fracture in the molecule can comprise a nick, a nick bordered by one or more unpaired nucleotides, or a gap. For ease of nomenclature, when a fractured hairpin is comprised of two strands, there may be a first strand and a second strand. The second strand may comprise (in linear order) a first region, a second region, a third region, and a fourth region. The first strand may comprise a portion of the sense or antisense region or all of the sense or antisense region of the molecule that after Dicer processing forms the double-stranded siRNA. The first region of the second strand can be capable of hybridizing or annealing to the first strand. The second region and the fourth regions of the second strand can be capable of hybridizing or annealing to each other. The third region of the second strand can correspond to the loop, which is physically positioned between the second region and the fourth region.

As used herein, the term “mismatch” includes a situation in which Watson-Crick base pairing does not take place between a nucleotide of a sense strand and a nucleotide of an antisense strand, where the non-base paired nucleotides are flanked by a duplex comprising base pairs in the 5′ direction of the mismatch beginning directly after (e.g., in the 5′ direction) the non-base paired nucleotides and in the 3′ direction of the mismatch beginning directly after (e.g., in the 3′ direction) the non-base paired nucleotides. An example of a mismatch would be an A across from a G, a C across from an A, a U across from a C, an A across from an A, a G across from a G, a C across from a C, and the like. Mismatches are also meant to include an abasic residue across from a nucleotide or modified nucleotide, an acyclic residue across from a nucleotide or modified nucleotide, a gap, or an unpaired loop. In its broadest sense, a mismatch as used herein includes any alteration at a given position that decreases the thermodynamic stability at or in the vicinity of the position where the alteration appears, such that the thermodynamic stability of the duplex at the particular position is less than the thermodynamic stability of a Watson-Crick base pair at that position.

As used herein, the term “nucleotide” is meant to refer to a ribonucleotide, a deoxyribonucleotide, or modified form thereof, as well as an analog thereof. Nucleotides include species that comprise purines (e.g., adenine, hypoxanthine, guanine, and their derivatives and analogs) and pyrimidines (e.g., cytosine, uracil, thymine, and their derivatives and analogs). Nucleotides are well known in the art. Nucleotide analogs can include nucleotides having modifications in the chemical structure of the base, sugar and/or phosphate, including, but not limited to, 5′-position pyrimidine modifications, 8′-position purine modifications, modifications at cytosine exocyclic amines, substitution of 5-bromo-uracil, and 2′-position sugar modifications (e.g., 2′ modifications). Such modifications include sugar-modified ribonucleotides in which the 2′-OH is replaced by a group such as an H, OR, R, halo, SH, SR, NH₂, NHR, NR₂, or CN, wherein R is an alkyl or aliphatic moiety. Nucleotide analogs are also meant to include nucleotides with bases such as inosine, queuosine, xanthine, sugars such as 2′-methyl ribose, non-natural phosphodiester linkages such as methylphosphonates, phosphorothioates, and peptides. Also, reference to a first nucleotide or nucleotide at a first position refers to the nucleotide at the 5′-most position of a duplex region, and the second nucleotide is the next nucleotide toward the 3′ end. In instances the duplex region extends to the end of the siRNA, the 5′ terminal nucleotide can be the first nucleotide.

As used herein, the term “nucleotide analogs” include nucleotides having modifications in the chemical structure of the base, sugar and/or phosphate, including, but not limited to, 5-position pyrimidine modifications, 8-position purine modifications, modifications at cytosine exocyclic amines, and substitution of 5-bromo-uracil; and 2′-position sugar modifications, including but not limited to, sugar-modified ribonucleotides in which the 2′-OH is replaced by a group such as an H, OR, R, halo, SH, SR, NH₂, NHR, NR₂, or CN, wherein R is an alkyl moiety as defined herein. Nucleotide analogs are also meant to include nucleotides with bases such as inosine, queuosine, xanthine, sugars such as 2′-methyl ribose, non-natural phosphodiester linkages such as methylphosphonates, phosphorothioates and peptides.

As used herein, the term “modified bases” is meant to refer to nucleotide bases such as, for example, adenine, guanine, cytosine, thymine, uracil, xanthine, inosine, and queuosine that have been modified by the replacement or addition of one or more atoms or groups. Some examples of types of modifications to the base moieties include but are not limited to, alkylated, halogenated, thiolated, aminated, amidated, or acetylated bases, individually or in combination. More specific examples include, for example, 5-propynyluridine, 5-propynylcytidine, 6-methyladenine, 6-methylguanine, N,N,-dimethyladenine, 2-propyladenine, 2-propylguanine, 2-aminoadenine, 1-methylinosine, 3-methyluridine, 5-methylcytidine, 5-methyluridine and other nucleotides having a modification at the 5 position, 5-(2-amino)propyl uridine, 5-halocytidine, 5-halouridine, 4-acetylcytidine, 1-methyladenosine, 2-methyladenosine, 3-methylcytidine, 6-methyluridine, 2-methylguanosine, 7-methylguanosine, 2,2-dimethylguanosine, 5-methylaminoethyluridine, 5-methyloxyuridine, deazanucleotides such as 7-deaza-adenosine, 6-azouridine, 6-azocytidine, 6-azothymidine, 5-methyl-2-thiouridine, other thio bases such as 2-thiouridine and 4-thiouridine and 2-thiocytidine, dihydrouridine, pseudouridine, queuosine, archaeosine, naphthyl and substituted naphthyl groups, any O— and N-alkylated purines and pyrimidines such as N6-methyladenosine, 5-methylcarbonylmethyluridine, uridine 5-oxyacetic acid, pyridine-4-one, pyridine-2-one, phenyl and modified phenyl groups such as aminophenol or 2,4,6-trimethoxy benzene, modified cytosines that act as G-clamp nucleotides, 8-substituted adenines and guanines, 5-substituted uracils and thymines, azapyrimidines, carboxyhydroxyalkyl nucleotides, carboxyalkylaminoalkyl nucleotides, and alkylcarbonylalkylated nucleotides. Modified nucleotides also include those nucleotides that are modified with respect to the sugar moiety, as well as nucleotides having sugars or analogs thereof that are not ribosyl. For example, the sugar moieties may be, or be based on, mannoses, arabinoses, glucopyranoses, galactopyranoses, 4′-thioribose, and other sugars, heterocycles, or carbocycles.

As used herein, the terms “off-target” and “off-target effects” are meant to refer to any instance where an siRNA, such as a synthetic siRNA or shRNA, is directed against a given target mRNA, but causes an unintended effect by interacting either directly or indirectly with another mRNA, a DNA, a cellular protein, or other moiety in a manner that reduces non-target protein expression. Often, this can happen when an siRNA interacts with non-target mRNA that has the same or similar polynucleotide sequence as the siRNA. For example, an “off-target effect” may occur when there is a simultaneous degradation of other non-target mRNA due to partial homology or complementarity between that non-target mRNA and the sense and/or antisense strand of the siRNA.

As used herein, the terms “overhang” or “overhang region” refer to terminal non-base pairing nucleotide(s) resulting from one strand or region extending beyond the terminus of the complementary strand to which the first strand or region forms a duplex. One or both of two polynucleotides or polynucleotide regions that are capable of forming a duplex through hydrogen bonding of base pairs may have a 5′ and/or 3′ end that extends beyond the 3′ and/or 5′ end of complementarity shared by the two polynucleotides or regions. The single-stranded region extending beyond the 3′ and/or 5′ end of the duplex is referred to as an overhang.

As used herein, the term “polynucleotide” is meant to refer to polymers of nucleotides linked together through internucleotide linkages. Also, a polynucleotide includes DNA, RNA, DNA/RNA, hybrids including polynucleotide chains of regularly and/or irregularly alternating deoxyribosyl moieties and ribosyl moieties (i.e., wherein alternate nucleotide units have an —OH, then and —H, then an —OH, then an —H, and so on at the 2′ position of a sugar moiety), and modifications of these kinds of polynucleotides. Also, polynucleotides include nucleotides with various modifications or having attachments of various entities or moieties to the nucleotide units at any position.

As used herein, the term “RNA interference” or “RNAi” are synonymous and refer to the process by which a polynucleotide, siRNA, shRNA or fractured shRNA comprising at least one ribonucleotide unit exerts an effect on a biological process. The process includes, but is not limited to, gene silencing by degrading mRNA, attenuating translation, interactions with tRNA, rRNA, hnRNA, miRNA, cDNA and genomic DNA, as well as methylation of DNA, and/or methylation or acetylation of proteins (e.g., histones) associated with DNA.

As used herein, the term “sense strand” is meant to refer to a polynucleotide or region that has the same nucleotide sequence, in whole or in part, as a target nucleic acid such as a messenger RNA or a sequence of DNA. The term “sense strand” includes the sense region of a polynucleotide that forms a duplex with an antisense region of another polynucleotide. Also, a sense strand can be a first polynucleotide sequence that forms a duplex with a second polynucleotide sequence on the same unimolecular polynucleotide that includes both the first and second polynucleotide sequences. As such, a sense strand can include one portion of a unimolecular siRNA that is capable of forming hairpin structure, such as an shRNA. When a sequence is provided, by convention, unless otherwise indicated, it is the sense strand or region, and the presence of the complementary antisense strand or region is implicit. The phrases “sense strand” and “sense region” are intended to be equivalent and are used interchangeably.

The sense region or sense strand may be part of a larger strand that comprises nucleotides other than sense nucleotides. For example, in the case of a unimolecular structure the larger strand would contain a sense region, an antisense region and a loop region, and might also contain overhang nucleotides and additional stem nucleotides that are complementary to other stem nucleotides, but not complementary to the target. In the case of a fractured hairpin, the sense region may be part of a strand that also comprises overhang nucleotides and/or a loop region and two other regions that are self-complementary.

As used herein, the term “siRNA” is meant to refer to a small inhibitory RNA duplex that induces gene silencing by operating within the RNA interference (“RNAi”) pathway. These siRNA are dsRNA that can vary in length, and can contain varying degrees of complementarity between the antisense and sense strands, and between the antisense strand and the target sequence. Each siRNA can include between 17 and 31 base pairs, more preferably between 18 and 26 base pairs, and most preferably 19 and 21 base pairs. Some, but not all, siRNA have unpaired overhanging nucleotides on the 5′ and/or 3′ end of the sense strand and/or the antisense strand. Additionally, the term “siRNA” includes duplexes of two separate strands, as well as single strands that can form hairpin structures comprising a duplex region, which may be referred to as short hairpin RNA (“shRNA”).

As used herein, the terms “shRNA” or “hairpins” are meant to refer to unimolecular siRNA comprised by a sense region coupled to an antisense region through a linker region. An shRNA may have a loop as long as, for example, 4 to 30 or more nucleotides. In some embodiments it may be preferable not to include any non-nucleotides moieties. The shRNA may also comprise RNAs with stem-loop structures that contain mismatches and/or bulges, micro-RNAs, and short temporal RNAs. RNAs that comprise any of the above structures can include structures where the loops comprise nucleotides, non-nucleotides, or combinations of nucleotides and non-nucleotides. Examples of shRNAs that comprise non-nucleotide loops are identified in U.S. Patent Application Publication No. US-2004-0058886-A1, the disclosure of which is incorporated by reference herein. The sense strand and antisense strand of an shRNA are part of one longer molecule or, in the case of fractured hairpins, two (or more) molecules that form a fractured hairpin structure.

Additionally, while the foregoing term definitions are intended to supplement the knowledge of one of ordinary skill in the art, not every term within this document has been defined. As such, the undefined terms are intended to be construed with the knowledge of one of ordinary skill in the art and/or the plain meaning of the term. Additionally, the foregoing terms are not intended to be limited by the examples provided therein, but are intended to be useful in understanding and practicing the invention as described herein.

B. ShRNA

In a first embodiment, the present invention can include a nucleic acid comprising a unimolecular RNA, such as an shRNA. The shRNA can be a unimolecular RNA that includes a first region, a loop region, and a second region. Preferably the first and second regions are at least substantially complementary to each other, and more preferably about 100% complementary to each other. More preferably, the first and second regions are each between 19 and 35 nucleotides in length. Most preferably, the first region and second region are between 26 and 32 nucleotides in length. Additionally, the first region and the second region within any unimolecular RNA of the invention can be the same length, or differ in length by less than about 5 bases, which as persons skilled in the art are aware can appear in a hairpin structure as a bulge or overhang. Any additional bases at the end of a first region or second region would be classified as part of the loop or overhang region. Furthermore, preferably the loop structure is about 4 to 30 nucleotides in length, and more preferably 7 nucleotides. In one particularly preferred embodiment, the loop is: 5′-AUAUGUG-3′ (SEQ. ID NO. 1) derived from the hsa-mir-17 sequence. Within any hairpin or fractured hairpin, preferable a plurality and more preferably all nucleotides are ribonucleotides.

A hairpin can be organized in either a left-handed hairpin (i.e., 5′-antisense-loop-sense-3′) or a right-handed hairpin (i.e., 5′-sense-loop-antisense-3′). Furthermore, the siRNA of the first embodiment may also contain overhangs at either the 5′ or 3′ end of either the sense strand or the antisense strand, depending upon the organization of the hairpin. Preferably, if there are any overhangs, they are on the 3′ end of the hairpin and comprise between 1 to 6 bases. The overhangs can be unmodified, or can contain one or more specificity or stabilizing modifications, such as a halogen or O-alkyl modification of the 2′ position, or internucleotide modifications such as phosphorothioate, phosphorodithioate, or methylphosphonate modifications. The overhangs can be ribonucleic acid, deoxyribonucleic acid, or a combination of ribonucleic acid and deoxyribonucleic acid.

Additionally, a hairpin can further comprise a phosphate group on the 5′-most nucleotide. The phosphorylation of the 5′-most nucleotide refers to the presence of one or more phosphate groups attached to the 5′ carbon of the sugar moiety of the 5′-terminal nucleotide. Preferably, there is only one phosphate group on the 5′ end of the region that will form the antisense strand following Dicer processing. In one aspect, a right-handed hairpin can include a 5′ end (i.e., the free 5′ end of the sense region) that does not have a 5′ phosphate group, or can have the 5′ carbon of the free 5′-most nucleotide of the sense region being modified in such a way that prevents phosphorylation. This can be achieved by a variety of methods including, but not limited to, addition of a phosphorylation blocking group (e.g., a 5′-O-alkyl group), or elimination of the 5′-OH functional group (e.g., the 5′-most nucleotide is a 5′-deoxy nucleotide). The 5′-deoxy chemistry is known to persons skilled in the art, and it is for example described in PCT/US04/10343, which published as WO 2004/090101 A2 on Oct. 21, 2004 and is incorporated by reference herein. In cases where the hairpin is a left-handed hairpin, preferably the 5′ carbon position of the 5′-most nucleotide is phosphorylated.

Hairpins that have stem lengths longer than 26 base pairs can be processed by Dicer such that some of the first region and/or second region may not be part of the resulting siRNA that facilitates mRNA degradation. Accordingly the first region, which may comprise sense nucleotides, and the second region, which may comprise antisense nucleotides, may also contain a stretch of nucleotides that are complementary (or at least substantially complementary to each other), but are or are not the same as or complementary to the target mRNA. While the stem of the shRNA can be composed of complementary or partially complementary antisense and sense strands exclusive of overhangs, the shRNA can also include the following: (1) the portion of the molecule that is distal to the eventual Dicer cut site contains a region that is substantially complementary/homologous to the target mRNA; and (2) the region of the stem that is proximal to the Dicer cut site (i.e., the region adjacent to the loop) is unrelated or only partially related (e.g., complementary/homologous) to the target mRNA. The nucleotide content of this second region can be chosen based on a number of parameters including but not limited to thermodynamic traits or profiles.

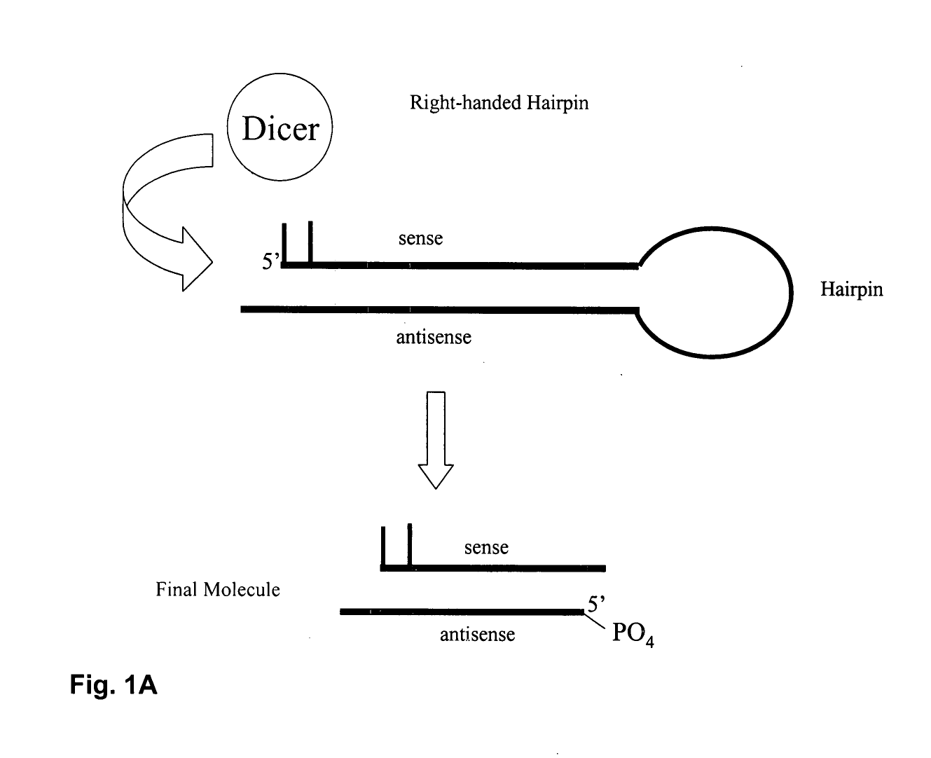

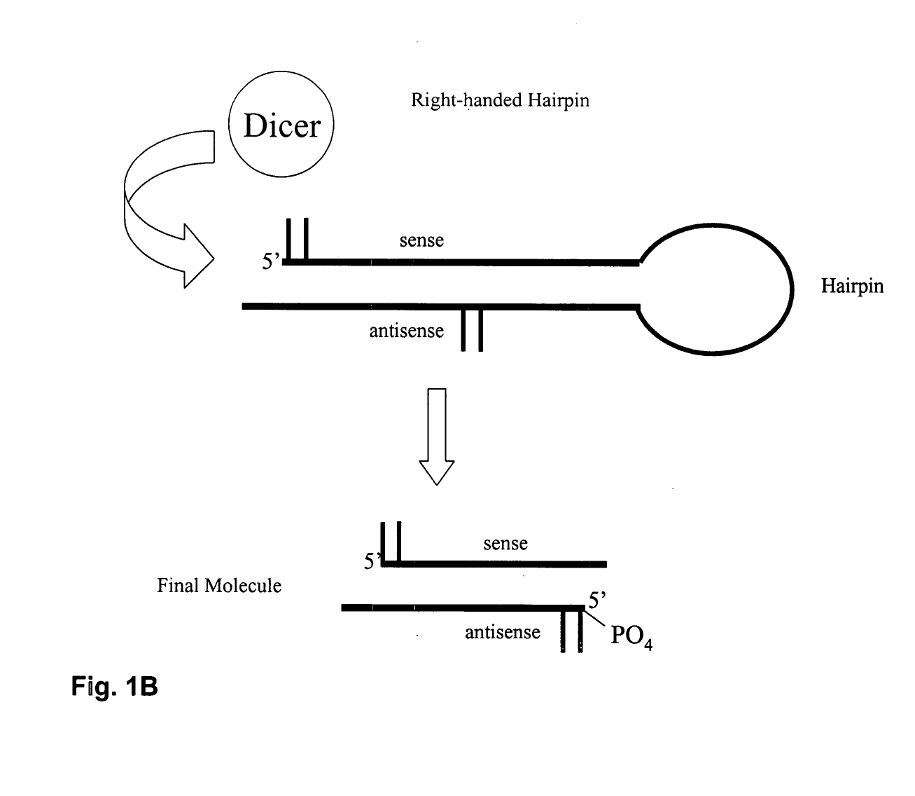

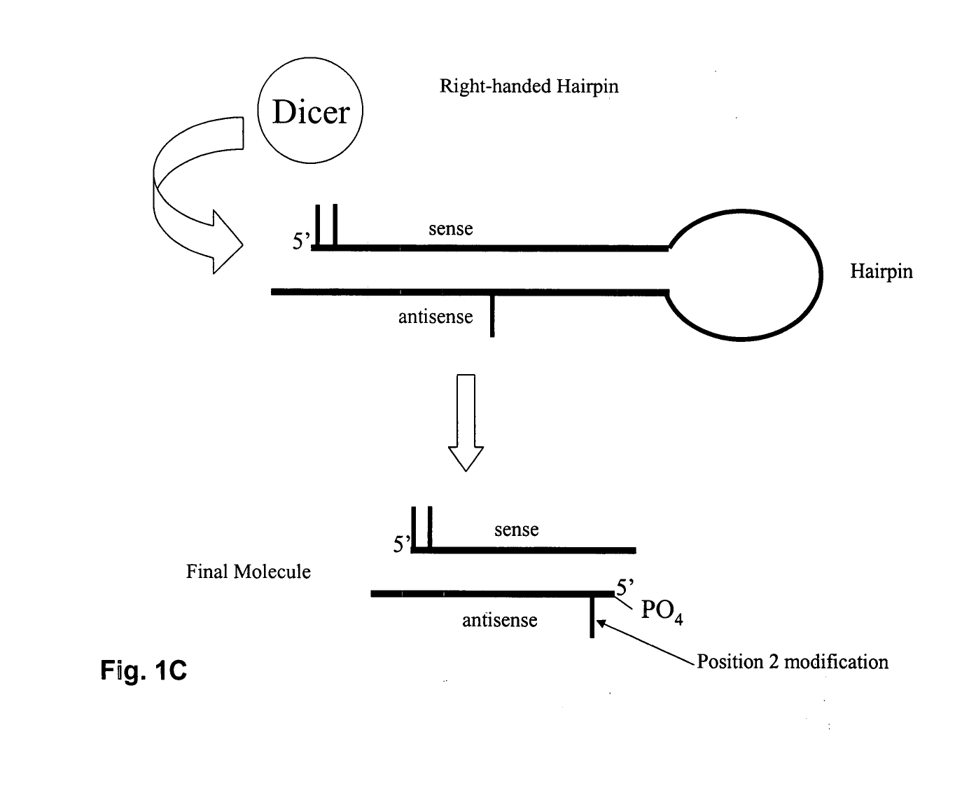

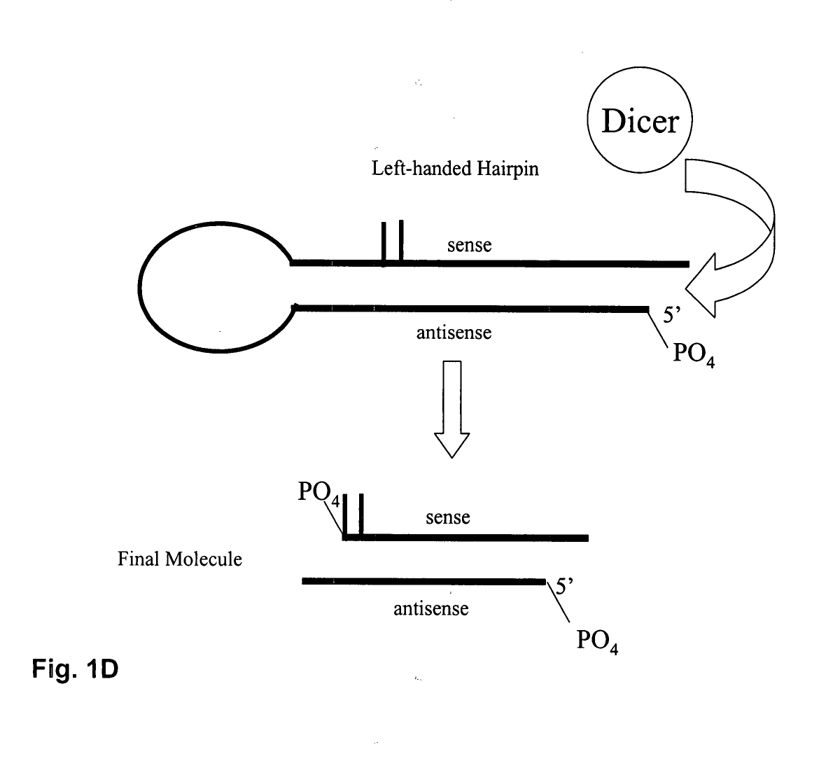

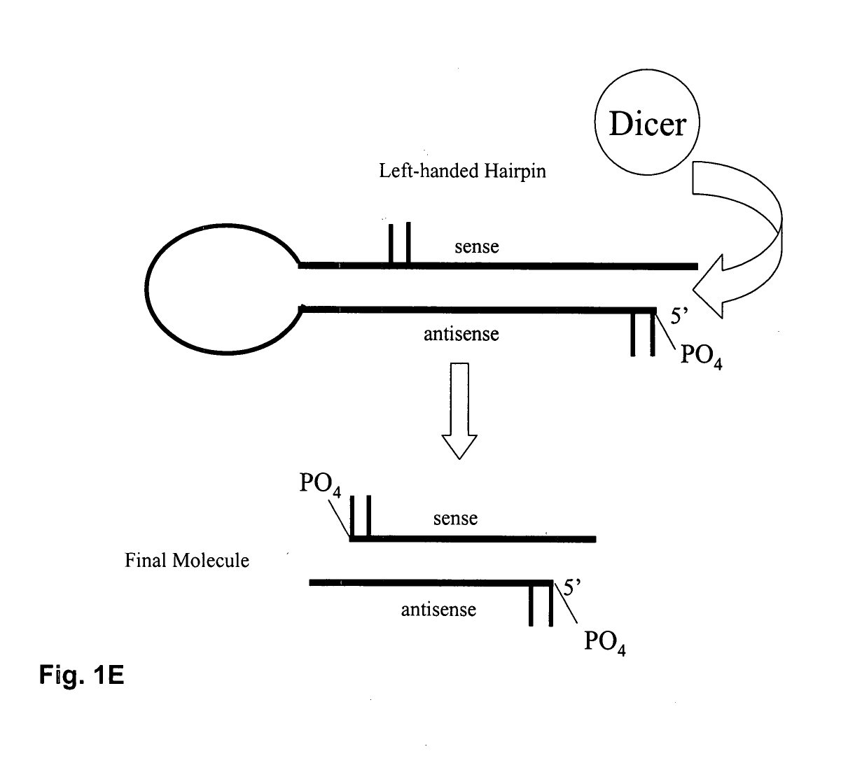

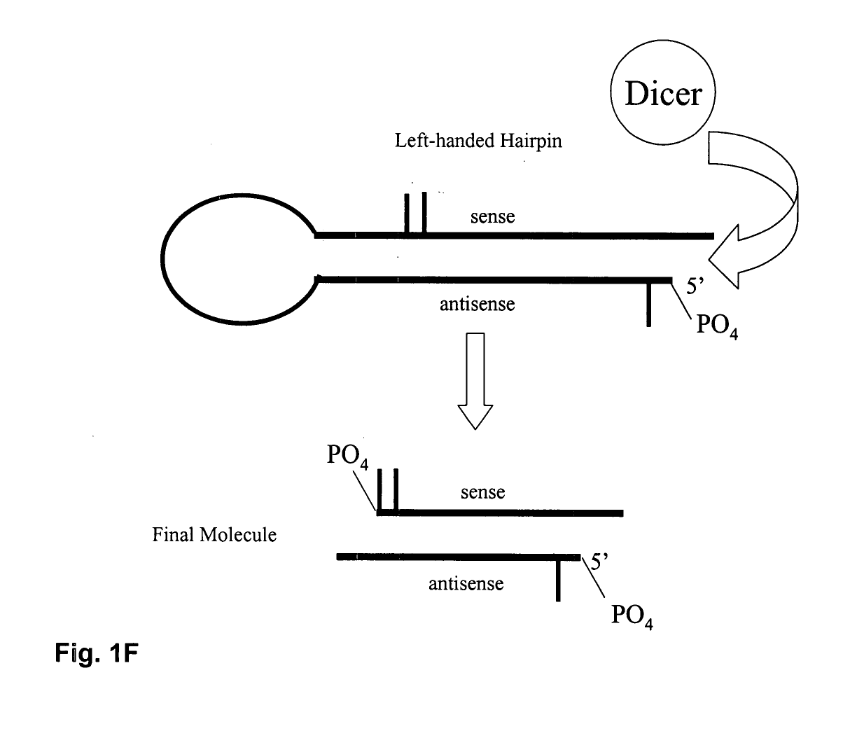

Optionally, additional modifications can be added to enhance shRNA stability (e.g., including but not limited to those described in the preceding paragraphs), functionality, and/or specificity. Such modified shRNAs can retain the modifications in the post-Dicer processed duplex. For instance, in cases in which the hairpin is a right handed hairpin (e.g., 5′-S-loop-AS-3′) containing 2-6 nucleotide overhangs on the 3′ end of the molecule, 2′-O-methyl modifications can be added to nucleotides at position 2, positions 1 and 2, or positions 1, 2, and 3 at the 5′ end of the hairpin (see FIG. 1A). Also, Dicer processing of hairpins with this configuration can retain the 5′ end of the sense strand intact, thus preserving the pattern of chemical modification in the post-Dicer processed duplex. Presence of a 3′ overhang in this configuration can be particularly advantageous since blunt ended molecules containing the prescribed modification pattern can be further processed by Dicer in such a way that the nucleotides carrying the 2′ modifications are removed. In cases where the 3′ overhang is present/retained, the resulting duplex carrying the sense-modified nucleotides can have highly favorable traits with respect to silencing specificity and functionality. Examples of preferred modification patterns are described in detail in U.S. patent application Ser. No. 11/019,831, filed Nov. 22, 2004, with pre-grant publication number 2005/0223427, International Patent application Serial No. PCT/US04/10343, which published as WO 2004/090105 A2 on Oct. 21, 2004, and International Patent application Serial No. PCT/US05/03365 filed on Feb. 4, 2005, the disclosures of which are incorporated by reference herein.

In another non-limiting example of modifications that can be applied to right handed hairpins, 2′-O-methyl modifications (or other 2′ modifications, including but not limited to other 2′-O-alkyl modifications) can be added to nucleotides at position 2, positions 1 and 2, or positions 1, 2, or 3 of the 5′ sense terminus of the hairpin, as well as to the first two (or just the second) nucleotide(s) of the region of the duplex that in the post-Dicer processed molecule represents the 5′ terminus of the antisense strand (see FIGS. 1B and 1C). The positions of internal chemical modifications can be determined in part by the length of the 3′ overhang. The general rules that define the position of the Dicer cut site, and thus the position of the modifications, are outlined in Vermeulen et al., RNA 11(5), 2005, which is incorporated by reference. Thus, in this example the antisense modifications may, for example, be located on the nucleotides that are complementary to sense nucleotides 19 and 20, 20 and 21, or 21 and 22.

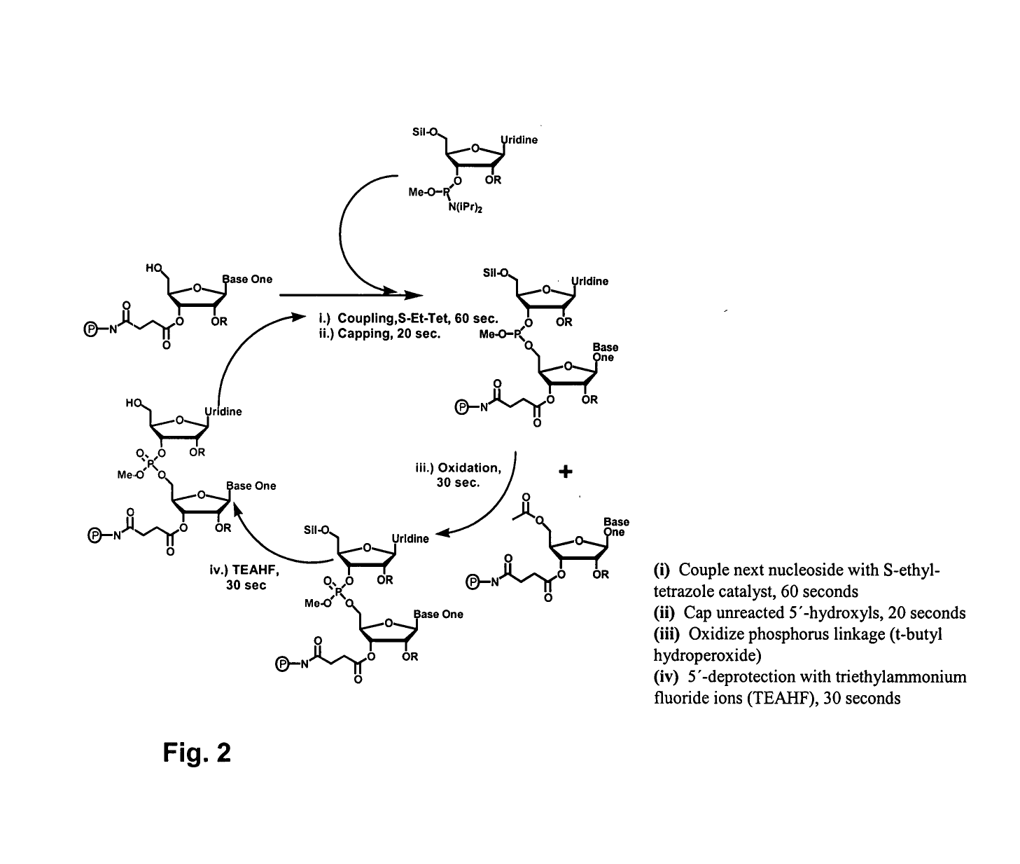

Similarly, in cases in which the hairpin is a left-handed hairpin (5′ AS-loop-S-3′, as illustrated in FIGS. 1D, 1E and 1F), 2′-O-alkyl modifications can be added to key positions within the molecule such that following Dicer digestion, the 2′-O-methyl groups are associated with: (1) the first and second nucleotides of the 5′ terminus of the sense strand; (2) the first and second nucleotides of the 5′ terminus of the sense strand, plus the first, and optionally second, nucleotides of the antisense strand; or (3) the first and second nucleotides of the 5′ terminus of the sense strand plus the second nucleotide of the antisense sense strand. Addition of chemical modifications in these nucleotide positions can greatly reduce the number of off-targeted genes produced by the sense and/or the sense and antisense strands and/or enhance functionality. Depictions of exemplary modifications in both the hairpin construct and the processed molecules appear in FIGS. 1A-1F.

Examples of modifications that can be added to right-handed hairpins to enhance hairpin specificity and functionality can include 5′ deoxy and 5′ blocking modifications. Previous studies have shown that for a strand to participate in RISC mediated RNAi, the 5′ carbon of the 5′ terminal nucleotide must be phosphorylated. As the sense strand of post-Dicer processed shRNA can potentially enter RISC and compete with the antisense (e.g., targeting) strand, modifications that prevent sense strand phosphorylation are valuable in minimizing off-target signatures. Thus, desirable chemical modifications that prevent phosphorylation of the 5′ carbon of the 5′-most nucleotide of right-handed shRNA of the invention can include, but are not limited to, modifications that: (1) add a blocking group (e.g., a 5′-O-alkyl) to the 5′ carbon; or (2) remove the 5′-hydroxyl group (e.g., 5′-deoxy nucleotides). Methods for generating 5′deoxy modified molecules are disclosed in International Patent Application Serial No. PCT/US05/03365, filed Feb. 4, 2005, and published as WO/2005/078094, the disclosure of which is incorporated by reference herein.

In addition to modifications that enhance specificity, modifications that enhance stability can also be added to the invention. In one embodiment, modifications comprising 2′-O-alkyl groups (or other 2′ modifications) can be added to one or more, and preferably all, pyrimidines (e.g., C and/or U nucleotides) of the sense strand. In another embodiment, 2′ F modifications (or other halogen modifications) can be added to one or more, and preferably all pyrimidines (e.g., C and/or U nucleotides) of the antisense strand. In yet a further embodiment, modifications comprising 2′-O-alkyl groups (or other 2′ modifications) can be added to one or more, preferably all, pyrimidines (e.g., C and/or U nucleotides) of the sense strand, plus 2′ F modifications (or other halogen modifications) can be added to one or more, preferably all pyrimidines (e.g., C and/or U nucleotides) of the antisense strand. Modifications such as 2′ F or 2′-O-alkyl of some or all of the Cs and Us of the antisense and/or sense strand/region, respectively, or the loop structure, can greatly enhance the stability of the shRNA molecules without appreciably altering target specific silencing. It should be noted that while these modifications enhance stability, it may be desirable to avoid the addition of these modification patterns to key positions in the hairpin in order to avoid disruption of RNAi (e.g., in and around the Dicer cleavage site).

Additionally stabilization modifications to the phosphate backbone may be included in the siRNAs in some embodiments of the present invention. For example, at least one phosphorothioate, phosphordithioate, and/or methylphosphonate may be substituted for the phosphate group at some or all 3′ positions of nucleotides in the shRNA backbone, or any particular subset of nucleotides (e.g., any or all pyrimidines in the sense and/or antisense strands of the oligonucleotide backbone), as well as in any overhangs, and/or loop structures present. Phosphorothioate and/or methylphosphonate analogues can arise from modification of the phosphate groups in the oligonucleotide backbone. In the phosphorothioate, the phosphate O⁻ can be replaced by a sulfur atom. In methylphosphonates, the oxygen can be replaced with a methyl group. These modifications may be used independently or in combination with the other modifications disclosed herein. Furthermore, in other embodiments, the compositions of the present invention can comprise at least one 2′-orthoester modification, wherein the 2′-orthoester modification is preferably a 2′-bis(hydroxy ethyl)-orthoester modification; 2′ orthoester modified siRNA exhibit enhanced nuclease resistance. All of the above modifications are described in detail in PCT/US04/10343, published as WO/2004/090105, which is incorporated by reference herein.

In a further embodiment, a label can be used in conjunction with the invention. Molecules of the invention containing labels can be useful as tracking agents, which would assist in detection of transfection, as well as detection of where in the cell the molecule is localized. Such labels can be added to one or more positions in the invention including the 5′ end of the molecule, the 3′ end of the molecule, the loop of the molecule, or internal positions associated with the sense and/or antisense regions, or a stem region of the molecules. Examples of commonly used labels include, but are not limited to, a fluorescent label, a radioactive label, a mass label or other well-known labels. The fluorescent label can be selected from the group consisting of TAMRA, BODIPY, Cy3, Cy5, fluoroscein, and Dabsyl. Alternatively, the fluorescent label can be any fluorescent label known in the art.

In other embodiments, any of the compositions of the present invention can further comprise a 3′ cap. The 3′ cap can be, for example, an inverted deoxythymidine.

In other embodiments of the present invention, any of the compositions can comprise a conjugate that enhances delivery, detection, function, specificity, or stability. The conjugate can be selected from the group consisting of amino acids, peptides, polypeptides, proteins, sugars, carbohydrates, lipids (e.g., cholesterol), polymers (e.g., polyethylene glycol), nucleotides, polynucleotides, and combinations thereof.

The above descriptions of the present invention may comprise sequences that were selected at random, or according to any rational design selection procedure. For example, the rational design algorithms are described in U.S. patent application Ser. No. 10/714,333, filed on Nov. 14, 2003, entitled “Functional and Hyperfunctional siRNA”; in International Patent Application Serial Number PCT/US2003/036787, which published on Jun. 3, 2004 as WO 2004/045543 A2, entitled “Functional and Hyperfunctional siRNA”; and in U.S. patent application Ser. No. 10/940,892, filed on Sep. 14, 2004, entitled “Methods and Compositions for Selecting siRNA of Improved Functionality,” having pre-grant publication number 2005/0255487. All of the algorithms and supporting disclosure of the aforementioned patent applications are incorporated by reference herein. Additionally, it may be desirable to select sequences in whole or in part based on average internal stability profiles (“AISPs”) or regional internal stability profiles (“RISPs”) that may facilitate access or processing by cellular machinery.

Embodiments of shRNA in accordance with the present invention can be synthesized by a variety of methods including but not limited to chemical synthesis, in vitro transcription, PCR-based techniques, or expression from a plasmid or viral vector. Such vectors can be stably maintained by integration into the host genome, or maintained as autonomous, self-replicating episomes. In instances where the shRNAs are created by expression from a viral vector, the preferred viral delivery system is one that is lentiviral in nature.

In the case of chemical synthesis, the preferred method of chemical synthesis is 2′-ACE synthesis. The synthesis is preferably carried out as an automated process on an appropriate machine. Several such synthesizing machines are known to those of skill in the art. Each nucleotide is added sequentially (3′- to 5′-direction) to a solid support-bound oligonucleotide. Although polystyrene supports are preferred, any suitable support can be used in the procedure. The first nucleoside at the 3′-end of the chain is covalently attached to a solid support. The nucleotide precursor, an activated ribonucleotide such as a phosphoramidite or H-phosphonate, and an activator such as a tetrazole, for example, S-ethyl-tetrazole (although any other suitable activator can be used) are added (step i in FIG. 2), coupling the second base onto the 5 ′-end of the first nucleoside. The support is washed and any unreacted 5′-hydroxyl groups are capped with an acetylating reagent such as, but not limited to, acetic anhydride or phenoxyacetic anhydride to yield unreactive 5′-acetyl moieties (step ii). The P(III) linkage is then oxidized to the more stable and ultimately desired P(V) linkage (step iii), using a suitable oxidizing agent such as, for example, t-butyl hydroperoxide or iodine and water. At the end of the nucleotide addition cycle, the 5′-silyl group is cleaved with fluoride ion (step iv), for example, using triethylammonium fluoride or t-butyl ammonium fluoride. The cycle is repeated for each subsequent nucleotide.

It should be emphasized that although FIG. 2 illustrates a phosphoramidite having a methyl protecting group, any other suitable group may be used to protect or replace the oxygen of the phosphoramidite moiety. For example, alkyl groups, cyanoethyl groups, or thio derivatives can be employed at this position. Further, the incoming activated nucleoside in step (i) can be a different kind of activated nucleoside, for example, an H-phosphonate, methyl phosphoramidite or a thiophosphoramidite. Also, it should be noted that the initial, or 3′, nucleoside attached to the support can have a different 5′ protecting group such as a dimethoxytrityl group, rather than a silyl group. Cleavage of the dimethoxytrityl group requires acid hydrolysis, as employed in standard DNA synthesis chemistry. Thus, an acid such as dichloroacetic acid (“DCA”) or trichloroacetic acid (“TCA”) is employed for this step alone. Apart from the DCA cleavage step, the cycle is repeated as many times as necessary to synthesize the polynucleotide desired.

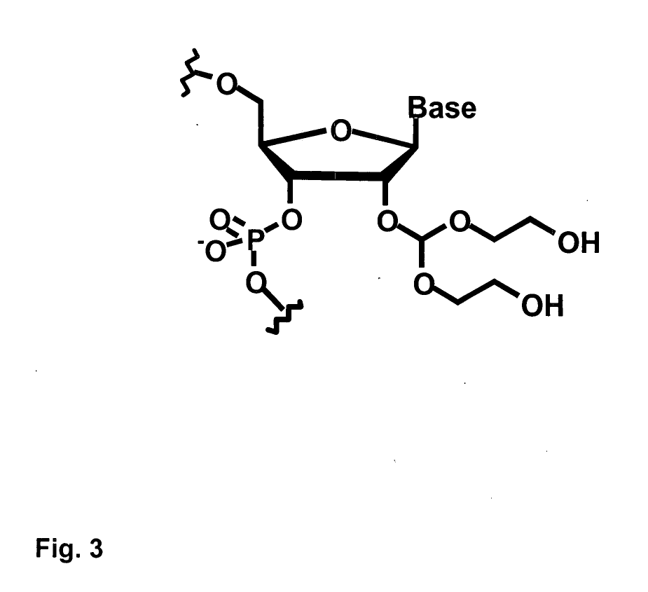

Following synthesis, the protecting groups on the phosphates, which are depicted as methyl groups in FIG. 2, but need not be limited to methyl groups, are cleaved in 30 minutes utilizing 1 M disodium-2-carbamoyl-2-cyanoethylene-1,1-dithiolate trihydrate (“dithiolate”) in DMF (“dimethylformamide”). The deprotection solution is washed from the solid support bound oligonucleotide using water. The support is then treated with 40% methylamine for 20 minutes at 55° C. This releases the RNA oligonucleotides into solution, deprotects the exocyclic amines and removes the acetyl protection on the 2′-ACE groups. The oligonucleotides can be analyzed by anion exchange HPLC at this stage. The 2′-orthoester groups are the last protecting groups to be removed, if removal is desired. The structure of the 2′-ACE protected RNA immediately prior to 2′-deprotection is as represented in FIG. 3.

For automated procedures, solid supports having the initial nucleoside are installed in the synthesizing instrument. The instrument will contain all the necessary ancillary reagents and monomers needed for synthesis. Reagents are maintained under argon, since some monomers, if not maintained under an inert gas, can hydrolyze. The instrument is primed so as to fill all lines with reagent. A synthesis cycle can be designed that defines the delivery of the reagents in the proper order according to the synthesis cycle, delivering the reagents in the order specified. Once a cycle is defined, the amount of each reagent to be added is defined, the time between steps is defined, and washing steps are defined, synthesis is ready to proceed once the solid support having the initial nucleoside is added.

For the RNA analogs described herein, modification is achieved through three different general methods. The first, which is implemented for carbohydrate and base modifications, as well as for introduction of certain linkers and conjugates, employs modified phosphoramidites in which the modification is pre-existing. An example of such a modification can be the carbohydrate 2′-modified species (2′-F, 2′-NH₂, 2′-O-alkyl, etc.), wherein the 2′ orthoester is replaced with the desired modification. The 3′or 5′ terminal modifications can also be introduced such as fluoroscein derivatives, Dabsyl, cholesterol, cyanine derivatives or polyethylene glycol. Certain inter-nucleotide bond modifications can also be introduced via the incoming reactive nucleoside intermediate. Examples of the resultant internucleotide bond modification include but are not limited to methylphosphonates, phosphoramidates, phosphorothioates or phosphorodithioates.

Many modifiers can be employed using the same or similar cycles. Examples in this class would include, for example, 2-aminopurine, 5-methyl cytidine, 5-aminoallyl uridine, diaminopurine, 2-O-alkyl, multi-atom spacers, single monomer spacers, 2′-aminonucleosides, 2′-fluoro nucleosides, 5-iodouridine, 4-thiouridine, acridines, 5-bromouridine, 5-fluorocytidine, 5-fluorouridine, 5-iodouridine, 5-iodocytidine, 5-biotin-thymidine, 5-fluoroscein-thymidine, inosine, pseudouridine, abasic monomer, nebularane, deazanucleoside, pyrene nucleoside, azanucleoside, and the like. Often the rest of the steps in the synthesis would remain the same with the exception of modifications that introduce substituents that are labile to standard deprotection conditions. Here modified conditions would be employed that do not affect the substituent. Second, certain internucleotide bond modifications require an alteration of the oxidation step to allow for their introduction. Examples in this class include phosphorothioates and phosphorodithioates, wherein oxidation with elemental sulfur or another suitable sulfur transfer agent may be required. Third, certain conjugates and modifications are introduced by a “post-synthesis” process, wherein the desired molecule is added to the biopolymer after solid phase synthesis is complete. An example of this would be the addition of polyethylene glycol to a pre-synthesized oligonucleotide that contains a primary amine attached to a hydrocarbon linker. Attachment in this case can be achieved by using a N-hydroxy-succinimidyl ester of polyethylene glycol in a solution phase reaction.

While this outlines the most preferred method for synthesis of synthetic RNA and its analogs, any nucleic acid synthesis method currently known or developed in the future that is capable of assembling these molecules could be employed in their assembly. Examples of alternative methods include 5′-DMT-2′-TBDMS and 5′-DMT-2′-TOM synthesis approaches. Also, some 2′-O-methyl, 2′-F and backbone modifications can be introduced in transcription reactions using modified and wild type T7 and SP6 polymerases.

While the preferred form of the invention is a hairpin comprising a single stranded oligonucleotide, the inventors recognize that chemical synthesis of unimolecular molecules of this length (e.g., >60 nucleotides) is challenging. For this reason, alternative approaches that take into consideration: (1) the efficiency of current RNA synthesis technologies; (2) the thermodynamics of duplex formation; and (3) the subtleties of Dicer processing, have been conceived. One preferred method involves the synthesis of a “fractured hairpin.”

C. Fractured Hairpin ShRNA

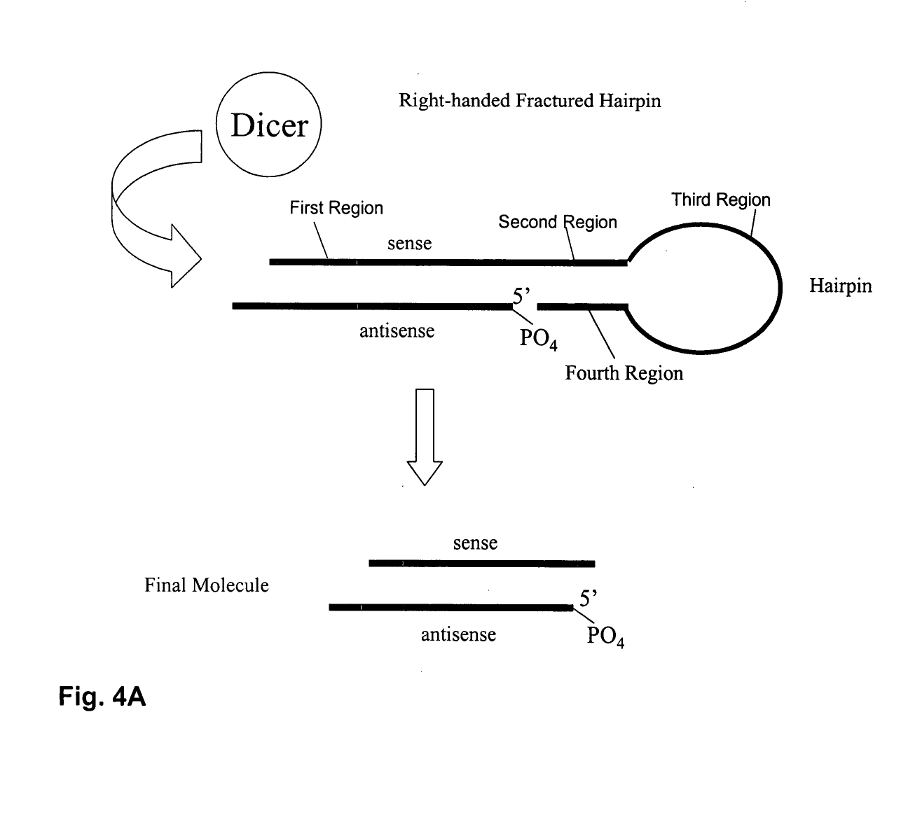

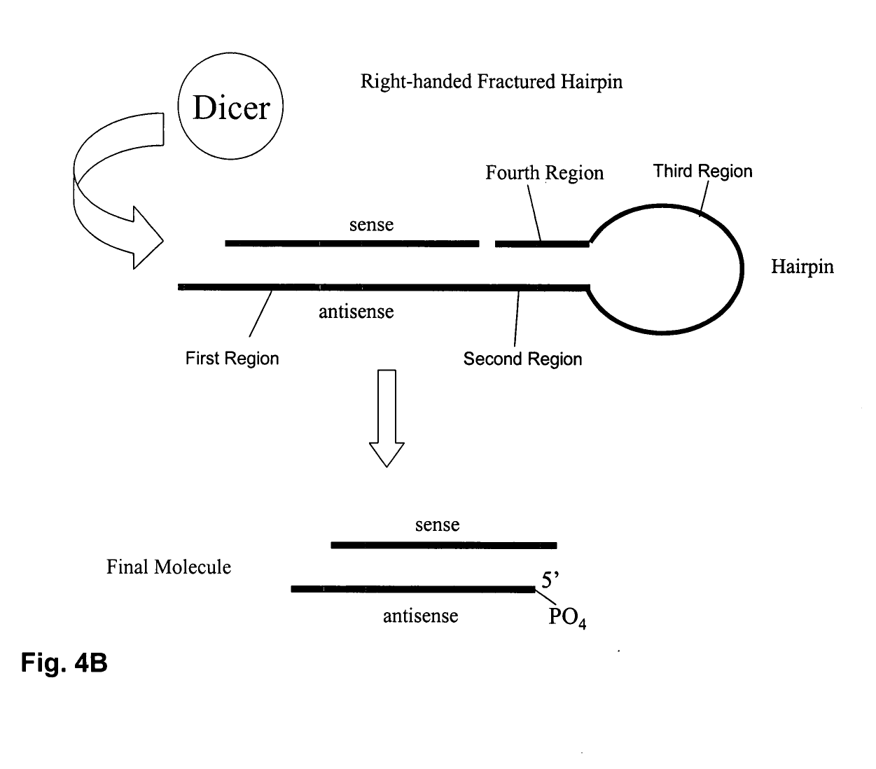

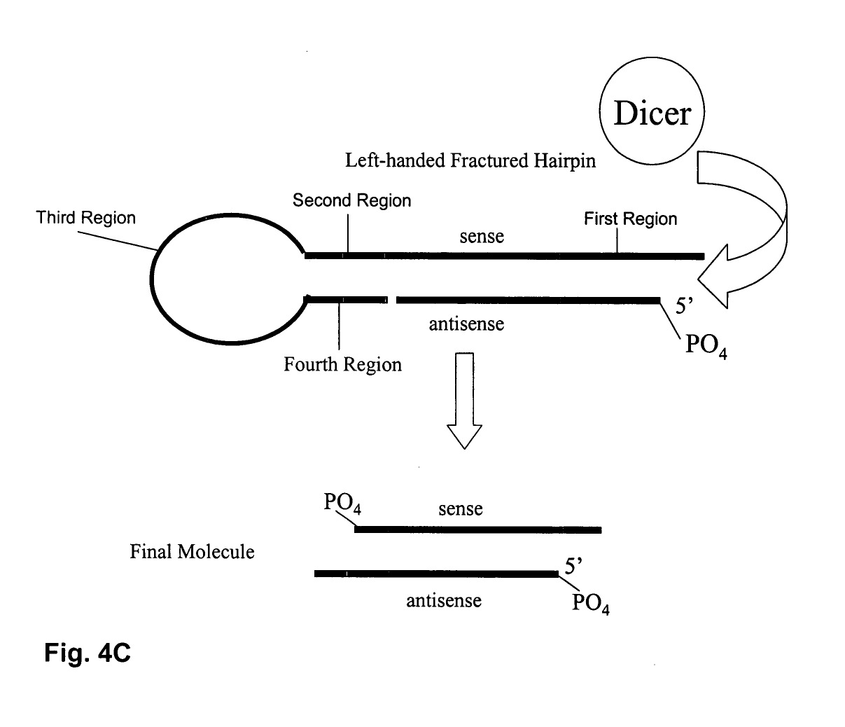

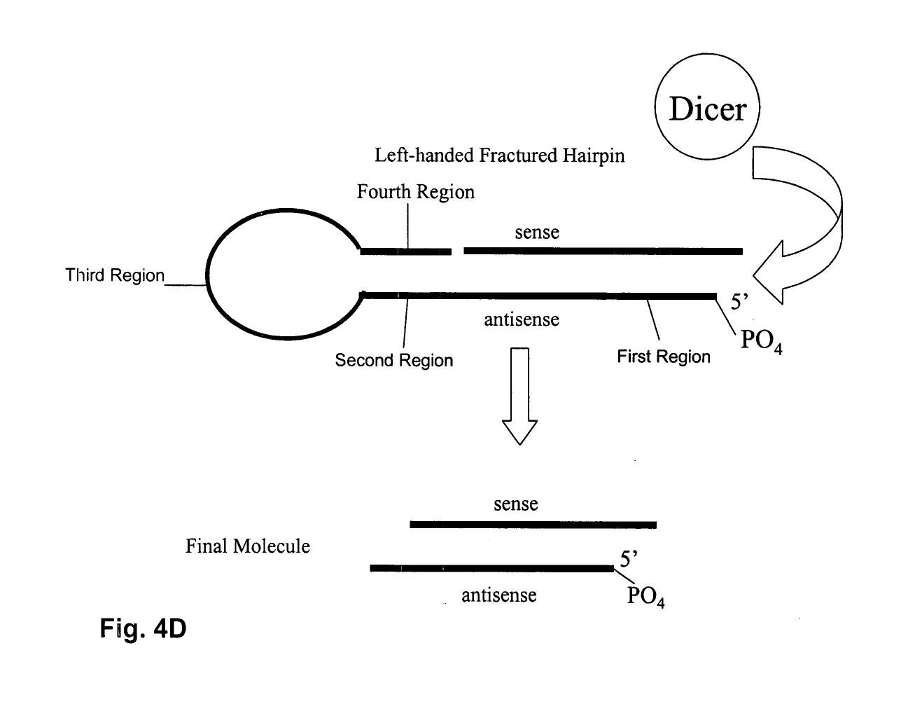

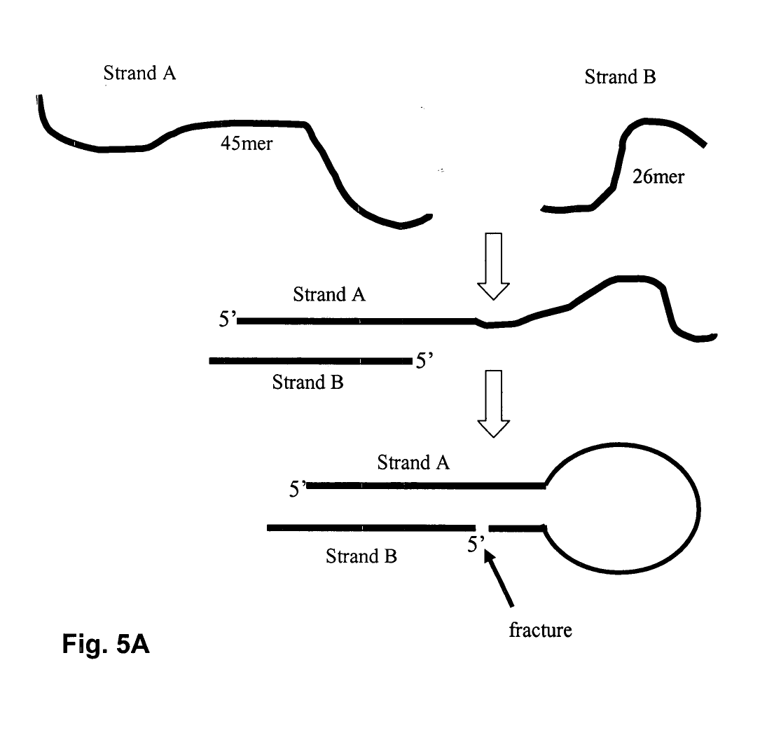

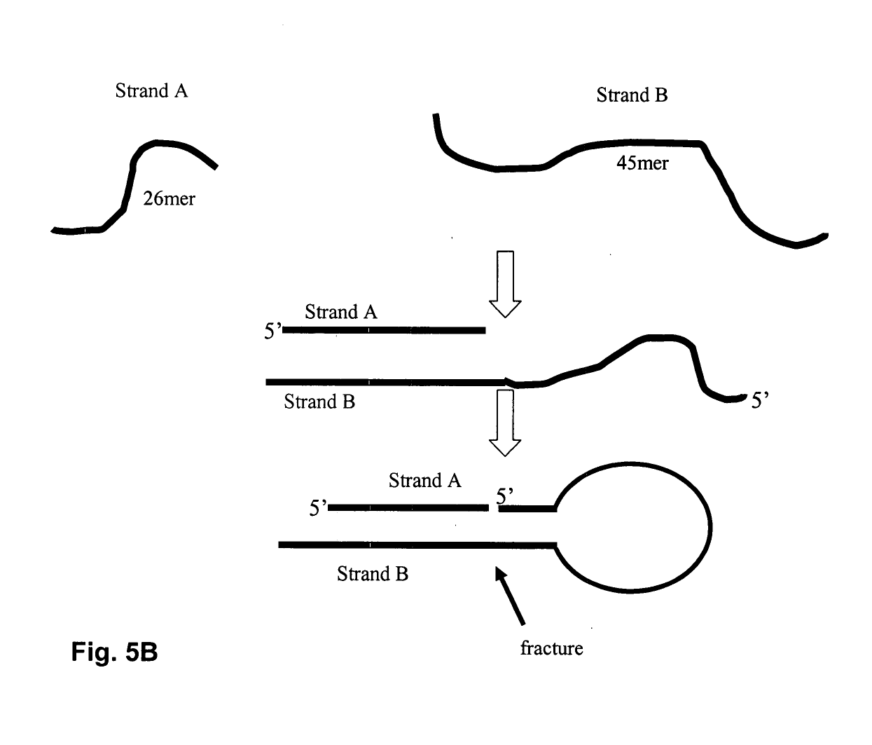

The fractured hairpins or sfhRNA, which are part of a second embodiment of the present invention can comprise two or more separate strands. In a preferred form of a fractured hairpin, the hairpin can comprise two strands (e.g., one long, one short) that anneal into a hairpin (see FIGS. 4A-4D). The length of each strand can be determined by a variety of factors including but not limited to: (1) the relative efficiency of the synthesis methodology; and (2) the desired position of Dicer cleavage. Taking into consideration the efficiency of synthesis, if the desired length of a unimolecular molecule is about 71 nucleotides, and the synthesis technology provides desired yields of oligonucleotides as long as about 45 nucleotides, one non-limiting example of a fractured hairpin can comprise two individual strands, with a 45-mer (e.g., Strand A), and a 26-mer (e.g., Strand B). As shown in FIG. 5A, a region of Strand A can be substantially complementary to Strand B. Furthermore, a portion of Strand A can comprise a region that is capable of annealing with an additional region of Strand A. Thus, mixing of strands A and B under conditions that allow strand annealing can lead to the generation of a fractured hairpin (i.e., a hairpin that contains a break/nick, or gap) that can be used for gene silencing. In this example the sense region and antisense regions are each 31 base pairs long.

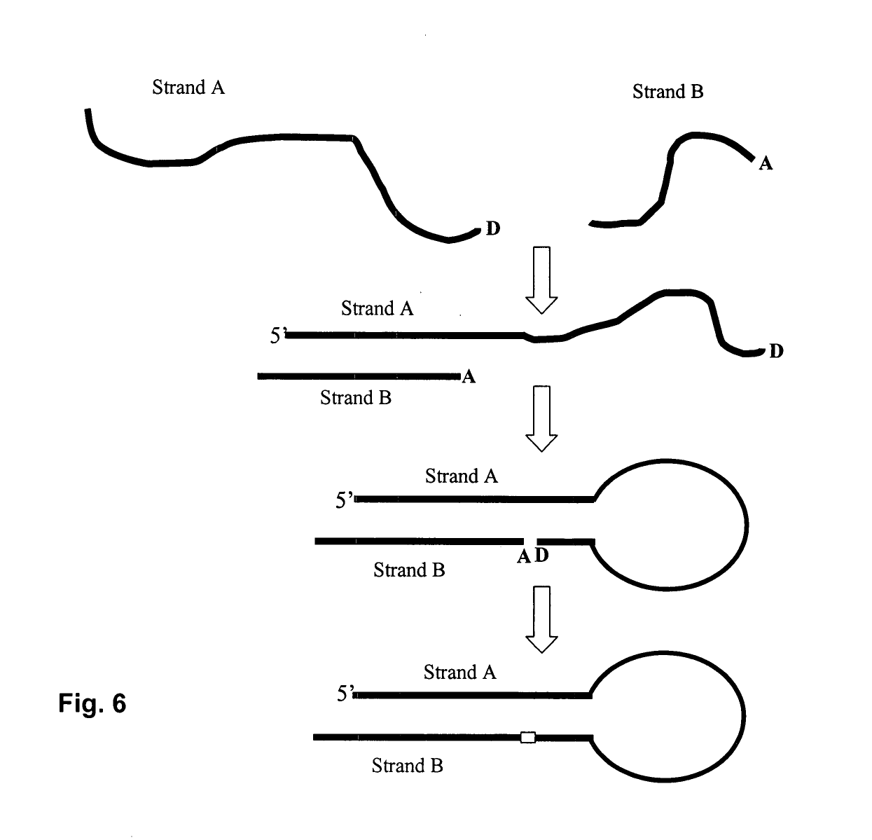

In a modification of the fractured hairpin preparation technique, Strand A and Strand B can be modified with a standard chemical donor-acceptor group or pair (see FIG. 6). In one non-limiting example, the 3′ end of Strand A (e.g., the longer strand) is modified with a donor group while the 5′ end of Strand B (e.g., the shorter strand) can be modified with an acceptor group. These two strands are then mixed under conditions that allow strand annealing to occur, thus placing the Donor and Acceptor groups in close enough proximity can enable interaction and subsequent ligation of the two strands. These procedures can generate a “modified hairpin,” which contains a non-conventional internucleotide linkage (i.e., a linkage that is not a phosphodiester linkage). In a second, non-limiting alternative, the acceptor can be associated with the 3′ end of Strand A, and the donor can be associated with the 5′ end of Strand B. Art recognized donor-acceptor groups for chemical RNA ligation that would be applicable under these conditions include but are not limited to those described in the following list: Amine/carboxylic acid plus activator (e.g., carbodiimide, EEDQ, etc.); Amine/carboxylic acid halide (e.g., chloride, bromide); Amine/carboxylic acid anhydride; Amine/active carboxylic acid ester (e.g., N-hydroxysuccinimidyl, p-nitrophenyl, pentafluorophenyl, N-hydroxybenzotriazolyl, etc.); Amine/imidoester (e.g., methyl imidate, etc.); Amine/carboxylic acid azide; Amine/carboxylic acid azolide (e.g., imidazolide, triazolide, etc.); Amine/phosphoric acid azolide (e.g., imidazolide, triazolide. etc.); Amine/carbonyl (e.g., aldehyde, ketone); Amine/acrylamide (e.g., [Michael addition reaction]); Hydrazide/carbonyl (e.g., aldehyde, ketone); Hydrazine/carbonyl (e.g., aldehyde, ketone); Hydroxylamine/carbonyl (e.g., aldehyde, ketone); Thiol/haloalkane (e.g., chloride, bromide, iodide); Thiol/haloacetamide (e.g., chloride, bromide, iodide); Thiol/maleimide; Thiol/disulfide (e.g., pyridyl disulfide, dithiopropionic acid); Thiol/thioester; Thiol/sulfonate that is alkyl or aryl (e.g., methanesulfonate, p-toluenesulfonate, trifluoromethanesulfonate, etc.); Hydroxyl/sulfonate that is alkyl or aryl (e.g., methanesulfonate, p-toluenesulfonate, trifluoromethanesulfonate, etc.); Amine/sulfonate that is alkyl or aryl (e.g., methanesulfonate, p-toluenesulfonate, trifluoromethanesulfonate, etc.); Thiophosphate/haloalkane (e.g., chloride, bromide, iodide); Thiophosphate/haloacetamide (chloride, bromide, iodide); Thiol/epoxide; Hydroxyl/epoxide; Amine/epoxide; Thiophosphate/epoxide; Diene/dieneophile (e.g., butadiene/maleimide. [Diels-Alder reaction]); Amine/hydroxyl plus formaldehyde (e.g., [Mannich reaction]); Amine/thiol (plus formaldehyde [Mannich reaction]); Amine/alkyne (plus formaldehyde [Mannich reaction]); and Amine/phenol (plus formaldehyde [Mannich reaction]). In yet another preferred embodiment, the two strands of the fractured hairpin are ligated together enzymatically. In this instance, the resulting molecule contains a normal internucleotide linkage (e.g., a phosphodiester linkage) and is therefore described as a conventional hairpin.

The length of the two strands in fractured hairpins can be important to consider. Preferably, the lengths of the two individual strands are determined such that the position of the fracture, nick, or gap does not occur in or near the loop portion of the molecule. In the absence of a donor-acceptor group, breaks in the loop may not form a hairpin. In the presence of donor-acceptor groups, the two strands can form a hairpin. Preferably, the two strands are of appropriate length such that the longer strand efficiently anneals with the smaller one, forms a loop, and then anneals with itself.

In another preferred embodiment, the length of the two strands can be determined by the cleavage properties of Dicer. Dicer cleaves long double stranded RNA and hairpins using two distinct RNase domains (RNase IIIa and RNase IIIb; Zhang et al. (2004) Single Processing Center Models for Human Dicer and Bacterial RNase III, Cell 118: 57-68). The position of cleavage is determined by a variety of factors including but not limited to, the length of the overhang on the 3′ end. In one non-limiting example, a right- or left-handed fractured hairpin is constructed whereby the position of the fracture, nick, or gap is at or downstream of the Dicer RNaseIIIb site of cleavage, or at (or upstream of) the Dicer RNase IIIa site. Positioning of the fracture, nick, or gap at this position can not only enhance the efficiency of Dicer cleavage, but can also direct Dicer to produce a more limited number of products. Specifically, when Dicer processes a long dsRNA or shRNA, it can typically generate between one and three different products of varying lengths (e.g., for long dsRNA or shRNA having 2 nt overhangs on the 3′ termini, Dicer typically generates products whose individual strands are between 22 and 24 nts in length). Depending upon the position of the fracture/nick/gap, the variability of cleavage products generated by Dicer can be narrowed. As a smaller subset of molecules have fewer chances of generating undesirable effects, fractured hairpins are more desirable from a therapeutic standpoint. Also, a fractured hairpin can include any of the stability, specificity, functional, or other modifications described above with respect to the shRNA.

Knowledge of the position of Dicer cleavage is also valuable in designing chemically modified fractured hairpin molecules that have improved specificity. Studies by a number of laboratories have demonstrated that siRNAs and shRNAs can down-regulate genes that contain sequences that are less than 100% homologous with the sequences comprising the siRNA or shRNA (see Jackson, A. L. et al. (2003). Expression profiling reveals off-target gene regulation by RNAi, Nature Biotechnology 21, 635-7). The inventors have recently identified a set of chemical modifications that can alter, minimize, or eliminate off-target effects of siRNA (U.S. patent application Ser. No. 11/019,831, filed Dec. 22, 2004, with pre-grant publication number 2005/0223427, the contents of which are incorporated herein). Unfortunately, preliminary studies have shown that incorporation of these modifications into hairpins (e.g., shRNAs) can, in some cases, lead to difficult to predict Dicer digestion patterns. In cases where the precision of Dicer cleavage is critical, fractured hairpins (containing the chemical modification of interest) can be substituted to generate predictable and highly functional molecules with minimal or altered off-target effects.

D. Modified ShRNA

According to a third embodiment, the present invention is directed to hairpin, fractured hairpin, and/or modified hairpin molecules that contain modified nucleotides. As described above, the modifications are included on specific nucleotides based on the duplex that will be formed after Dicer processing. Thus, for example the 2′ modifications may be distributed according to any one of the following patterns: (1) 2′ O-alkyl modifications on the nucleotides that will be the first and second sense nucleotides after Dicer processing; (2) 2′ O-alkyl modifications on the nucleotides that will be the first and second sense nucleotides and the second antisense nucleotide after Dicer processing; or (3) 2′ O-alkyl modifications on the nucleotides that will be the first and second sense nucleotides and the first and second antisense nucleotide after Dicer processing. In each of these types of molecules, preferably the 2′ O-alkyl modification is 2′O-methyl. The hairpins, fractured hairpins, and/or modified hairpins may also be designed so that the resulting duplex after Dicer processing has a phosphate group of the first antisense nucleotide. Thus, e.g., if the hairpin is oriented 5′ antisense region, loop and sense region, then the terminal 5′ nucleotide may have a phosphate group. The preferable size of these molecules and presence or absence of modifications may be defined in manners similar to as they were defined for the first two embodiments.

E. Gene Silencing

According to a fourth embodiment, the present invention can be directed to a method for inducing gene silencing, said method comprising exposing an shRNA and/or sfhRNA to a target nucleic acid or to a cell that is expressing or is capable of expressing said target nucleic acid. The shRNA may be defined according to the parameters of the first embodiment, and the sfhRNA may be defined according to the parameters of the second embodiment.

Because the invention is not dependent on the sequence of the bases, it can be applied to any sequence, regardless of its base composition, or the method by which the sequence was selected (e.g., randomly selected sequences and rationally designed sequences). Moreover the invention is applicable to a wide range of cell types, such as embryonic cells, oocytes, sperm cells, adipocytes, fibroblasts, myocytes, cardiomyocytes, endothelium, neurons, glia, blood cells, megakaryocytes, lymphocytes, macrophages, neutrophils, eosinophils, basophils, mast cells, leukocytes, granulocytes, keratinocytes, chondrocytes, osteoblasts, osteoclasts, hepatocytes and cells of the endocrine or exocrine glands and organisms, such as plants, animals, protozoa, bacteria, viruses, and fungi. The present invention is particularly advantageous for use in in vivo studies in mammals such as cattle, horse, goats, pigs, sheep, canines, rodents such as hamsters, mice, and rats, and primates such as, gorillas, chimpanzees, and humans. The present invention is most applicable for use for employing RNA interference in humans, tissues derived from humans, or cell lines derived from humans, particularly against the 45,000 genes of a human genome, and genes implicated in diseases including but not limited to diabetes, Alzheimer's, epilepsy, and cancer.

The molecules of the present invention may be administered to a cell by any method that is now known or that comes to be known and that from reading this disclosure, one skilled in the art would conclude would be useful with the present invention. For example, the siRNAs may be passively delivered to cells.

Passive uptake of molecules of the invention can be modulated, for example, by the presence of a conjugate such as a polyethylene glycol moiety or a cholesterol moiety at one or more termini or internal positions of the molecule and/or, in appropriate circumstances, a pharmaceutically acceptable carrier.

Other methods for delivery include, but are not limited to, transfection techniques employing DEAE-Dextran, calcium phosphate, cationic lipids/liposomes, microinjection, electroporation, immunoporation, and coupling of the siRNAs to specific conjugates or ligands such as antibodies, peptides, antigens, or receptors, using forward or reverse transfection (“RTF”) protocols. In one preferred embodiment, the molecules of the invention are delivered to cells using the reverse transfection protocol described in U.S. Provisional Patent Application Ser. No. 60/630,320, which was filed on Nov. 22, 2004, U.S. patent applications having Ser. Nos. 11/283,481, 11/283,482, 11/283,483, and 11/283,484, which were all filed on Nov. 18, 2005, and all are incorporated herein by reference. Briefly, in this procedure, molecules of the invention are dried on a solid surface (e.g., the bottom of a well in a 96, 384, or 1536 well plate), solubilized by the addition of a carrier (e.g., a lipid transfection reagent), followed by the addition of the cell type(s) of choice for transfection.

Further, the method of assessing the level of gene silencing is not limited. Thus, the silencing ability of any given siRNA can be studied by one of any number of art tested procedures including but not limited to Northern analysis, Western Analysis, RT PCR, expression profiling, and others.

The shRNA and sfhRNAs of the present invention may be used in a diverse set of applications, including but not limited to basic research, drug discovery and development, diagnostics, and therapeutics. In research settings, the application can involve introduction of hairpin molecules into cells using either a reverse transfection or forward transfection protocol. For example, the present invention may be used to validate whether a gene product is a target for drug discovery or development. In this application, the mRNA that corresponds to a target nucleic acid sequence of interest is identified for targeted degradation. Inventive siRNAs, shRNAs, or sfhRNAs that are specific for targeting the particular gene are introduced into a cell or organism. The cell or organism is maintained under conditions allowing for the degradation of the targeted mRNA, resulting in decreased activity or expression of the gene. The extent of any decreased expression or activity of the gene is then measured, along with the effect of such decreased expression or activity, and a determination is made that if expression or activity is decreased, then the nucleic acid sequence of interest is an agent for drug discovery or development. In this manner, phenotypically desirable effects can be associated with RNA interference of particular target nucleic acids of interest and in appropriate cases toxicity and pharmacokinetic studies can be undertaken and therapeutic preparations developed.

In another application of using the modified or unmodified siRNAs, shRNAs, and/or sfhRNAs of the invention, cells are transfected with pools of molecules of the invention or individual molecules of the invention that constitute the pools. In this way, a user is able to identify the most functional siRNAs, shRNAs, and/or sfhRNAs or combination of siRNAs against an individual target.

In yet another application, siRNA and/or sfhRNAs can be directed against a particular family of genes (e.g., kinases), genes associated with a particular pathway(s) (e.g., cell cycle regulation), or entire genomes (e.g., the human, rat, mouse, C. elegans, or Drosophila genome). Knockdown of each gene of the collection with molecules of the invention would enable researchers to assess quickly the contribution of each member of a family of genes, or each member of a pathway, or each gene in a genome, to a particular biological function or event. As one example of this sort of application, individuals who are interested in identifying one or more host (e.g., human) genes that contribute to the ability of e.g., the HIV virus to infect human cells, can plate molecules of the invention directed against the entire human genome in a RTF format. Following lipoplex formation, cells that are susceptible to HIV infection (e.g., JC53 cells) are added to each well for transfection. After culturing the cells for a period of 24-48 hours, the cells in each well could be subjected to a lethal titer of the HIV virus. Following an appropriate incubation period necessary for infection, plates could be examined to identify which wells contain living cells. Wells that contain living cells (or a substantially larger number of living cells than controls) can be used to identify a host gene that is necessary for viral infection, replication, and/or release. In this way, one is able to identify host genes that play a role in pathogen infection.

In yet another application, cells transfected with molecules of the invention are used to assess a particular gene's (e.g., target's) contribution to exclusion of a drug from cells. In one non-limiting example, cells are reverse transfected on RTF plates that contain shRNA and fractured hairpins directed against all known members of the human genome, shRNA and fractured hairpins directed against a particular family of genes (e.g., kinases), or siRNA, shRNAs, and/or sfhRNAs directed against genes of a particular pathway (e.g., the ADME-tox pathways). Subsequently, cells are treated with a particular compound (e.g., a potential therapeutic compound) and the ability of cells to, e.g., retain, excrete, metabolize, or adsorb that compound can be measured and compared with, cells that have not been treated with the molecule(s) of the invention. In this way, a researcher can identify one or more host genes that play a role in the pharmacokinetics of the compound under study.

In yet another application, cells transfected with molecules of the invention are used to validate the target of one or more biologically relevant agents (e.g., a drug). For instance, if a particular drug is believed to target a particular protein and induce a particular phenotype, the action of the drug can be validated by targeting that protein with a molecule of the invention. If the siRNA, shRNAs, and/or sfhRNAs induces the same phenotype as the drug, then the target is validated. If the molecule of the invention fails to induce the same phenotype, then these experiments would question the validity of the proposed protein as the drug target.

In yet another application, two or more molecules of the invention and targeting two or more distinct targets can be used to identify and study synthetic lethal pairs.

In yet another application, shRNA and/or sfhRNA of the invention can be used to target transcripts containing single nucleotide polymorphisms (“SNPs”) to facilitate and assess the contribution of a particular SNP to a phenotype, a biological function, a disease state, or event.

In yet another application, molecules of the invention can be used to target a gene(s) whose knockdown is known to induce a particular disease state. In this way, it is possible to facilitate study of that particular disease without: (1) the risk of knocking down the expression of additional genes; or (2) costly generation of e.g., knockout animals.

In all of the applications described above, the applications can be employed in such a way as to knock down one or multiple genes in a single well.

The present invention may also be used in RNA interference applications that induce transient or permanent states of disease or disorder in an organism by, for example, attenuating the activity of a target nucleic acid of interest believed to be a cause or factor in the disease or disorder of interest. Increased activity of the target nucleic acid of interest may render the disease or disorder worse, or tend to ameliorate or to cure the disease or disorder of interest, as the case may be. Likewise, decreased activity of the target nucleic acid of interest may cause the disease or disorder, render it worse, or tend to ameliorate or cure it, as the case may be. Target nucleic acids of interest can comprise genomic or chromosomal nucleic acids or extrachromosomal nucleic acids, such as viral nucleic acids.

Still further, the present invention may be used in RNA interference applications, such as diagnostics, prophylactics, and therapeutics including use of the compositions in the manufacture of a medicament in animals, preferably mammals, more preferably humans in the treatment of diseases, or over or under expression of a target. Preferably, the disease or disorder is one that arises from the malfunction of one or more proteins, the disease or disorder of which is related to the expression of the gene product of the one or more proteins. For example, it is widely recognized that certain cancers of the human breast are related to the malfunction of a protein expressed from a gene commonly known as the “bcl-2” gene. A medicament can be manufactured in accordance with the compositions and teachings of the present invention, employing one or more siRNAs directed against the bcl-2 gene, and optionally combined with a pharmaceutically acceptable-carrier, diluent and/or adjuvant, which medicament can be used for the treatment of breast cancer. Applicants have established the utility of the methods and compositions in cellular models. Methods of delivery of polynucleotides, such as siRNAs/shRNAs/sfhRNAs, to cells within animals, including humans, are well known in the art. Any delivery vehicle now known in the art, or that comes to be known, and has utility for introducing polynucleotides, such as siRNAs/shRNAs/sfhRNAs, to animals, including humans, is expected to be useful in the manufacture of a medicament in accordance with the present invention, so long as the delivery vehicle is not incompatible with any modifications that may be present a composition made according to the present invention. A delivery vehicle that is not compatible with a composition made according to the present invention is one that reduces the efficacy of the composition by greater than 95% as measured against efficacy in cell culture.

Animal models exist for many, many disorders, including, for example, cancers, diseases of the vascular system, inborn errors or metabolism, and the like. It is within ordinary skill in the art to administer nucleic acids to animals in dosing regimens to arrive at an optimal dosing regimen for particular disease or disorder in an animal such as a mammal, for example, a mouse, rat or non-human primate. Once efficacy is established in the mammal by routine experimentation by one of ordinary skill, dosing regimens for the commencement of human trials can be arrived at based on data arrived at in such studies.

Dosages of medicaments manufactured in accordance with the present invention may vary from micrograms per kilogram to hundreds of milligrams per kilogram of a subject. As is known in the art, dosage will vary according to the mass of the mammal receiving the dose, the nature of the mammal receiving the dose, the severity of the disease or disorder, and the stability of the medicament in the serum of the subject, among other factors well known to persons of ordinary skill in the art.

For these applications, an organism suspected of having a disease or disorder that is amenable to modulation by manipulation of a particular target nucleic acid of interest is treated by administering shRNAs or sfhRNAs of the invention. Results of the shRNA or sfhRNA treatment may be ameliorative, palliative, prophylactic, and/or diagnostic of a particular disease or disorder. Preferably, the siRNAs, shRNAs, or sfhRNAs are administered in a pharmaceutically acceptable manner with a pharmaceutically acceptable carrier or diluent.

Therapeutic applications of the present invention can be performed with a variety of therapeutic compositions and methods of administration. Pharmaceutically acceptable carriers and diluents are known to persons skilled in the art Methods of administration to cells and organisms are also known to persons skilled in the art. Dosing regimens, for example, are known to depend on the severity and degree of responsiveness of the disease or disorder to be treated, with a course of treatment spanning from days to months, or until the desired effect on the disorder or disease state is achieved. Chronic administration of shRNAs or fractured shRNAs of the invention may be required for lasting desired effects with some diseases or disorders. Suitable dosing regimens can be determined by, for example, administering varying amounts of one or more shRNA or fractured shRNA of the invention in a pharmaceutically acceptable carrier or diluent by a pharmaceutically acceptable delivery route, and amount of drug accumulated in the body of the recipient organism can be determined at various times following administration. Similarly, the desired effect (for example, degree of suppression of expression of a gene product or gene activity) can be measured at various times following administration of the nucleic acid, and this data can be correlated with other pharmacokinetic data, such as body or organ accumulation. Those of ordinary skill can determine optimum dosages, dosing regimens, and the like. Those of ordinary skill may employ EC50 data from in vivo and in vitro animal models as guides for human studies.

Further, the shRNA and/or sfhRNA can be administered in a cream or ointment topically, an oral preparation such as a capsule or tablet or suspension or solution, and the like. The route of administration may be intravenous, intramuscular, dermal, subdermal, cutaneous, subcutaneous, intranasal, oral, rectal, by eye drops, or by tissue implantation of a device that releases the nucleic acid at an advantageous location, such as near an organ or tissue or cell type harboring a target nucleic acid of interest.

Having described the invention with a degree of particularity, examples will now be provided. These examples are not intended to and should not be construed to limit the scope of the claims in any way. Although the invention may be more readily understood through reference to the following examples, they are provided by way of illustration and are not intended to limit the present invention unless specified.

### EXAMPLES

### Example 1

### RNA Synthesis

The polynucleotides of the present invention may be synthesized by any method that is now known or that comes to be known, and that from reading this disclosure a person of ordinary skill in the art would appreciate would be useful for synthesizing the molecules of the present invention. For example, shRNA and or shRNA may be chemically synthesized using compositions of matter and methods described in Scaringe, S. A. (2000) “Advanced 5′-silyl-2′-orthoester approach to RNA oligonucleotide synthesis,” Methods Enzymol. 317, 3-18; Scaringe, S. A. (2001) “RNA oligonucleotide synthesis via 5′-silyl-2′-orthoester chemistry,” Methods 23, 206-217; Scaringe, S. and Caruthers, M. H. (1999) U.S. Pat. No. 5,889,136; Scaringe, S. and Caruthers, M. H. (1999) U.S. Pat. No. 6,008,400; Scaringe, S. (2000) U.S. Pat. No. 6,111,086; Scaringe, S. (2003) U.S. Pat. No. 6,590,093; which are all incorporated herein by reference. The synthesis method utilizes nucleoside base-protected 5′-O-silyl-2′-O-orthoester-3′-O-phosphoramidites to assemble the desired unmodified siRNA sequence on a solid support in the 3′ to 5′ direction. Briefly, synthesis of the required phosphoramidites begins from standard base-protected ribonucleosides (e.g., uridine, N4-acetylcytidine, N2-isobutyrylguanosine and N6-isobutyryladenosine). Introduction of the 5′-O-silyl and 2′-O-orthoester protecting groups, as well as the reactive 3′-O-phosphoramidite moiety is then accomplished in five steps, including: Simultaneous transient blocking of the 5′- and 3′-hydroxyl groups of the nucleoside sugar with Markiewicz reagent (e.g., 1,3-dichloro-1,1,3,3,-tetraisopropyldisiloxane [TIPS-Cl₂]) in pyridine solution {Markiewicz, W. T. (1979) “Tetraisopropyldisiloxane-1,3-diyl, a Group for Simultaneous Protection of 3′- and 5′-Hydroxy Functions of Nucleosides,” J. Chem. Research(S), 24-25}, followed by chromatographic purification; Regiospecific conversion of the 2′-hydroxyl of the TIPS-nucleoside sugar to the bis(acetoxyethyl)orthoester [ACE derivative] using tris(acetoxyethyl)-orthoformate in dichloromethane with pyridinium p-toluenesulfonate as catalyst, followed by chromatographic purification; Liberation of the 5′- and 3′-hydroxyl groups of the nucleoside sugar by specific removal of the TIPS-protecting group using hydrogen fluoride and N,N,N″N′-tetramethylethylene diamine in acetonitrile, followed chromatographic purification; Protection of the 5′-hydroxyl as a 5′-O-silyl ether using benzhydroxy-bis(trimethylsilyloxy)silyl chloride [BzH-Cl] in dichloromethane, followed by chromatographic purification; and Conversion to the 3′-O-phosphoramidite derivative using bis(N,N-diisopropylamino)methoxyphosphine and 5-ethylthio-1H-tetrazole in dichloromethane/acetonitrile, followed by chromatographic purification.

The phosphoramidite derivatives are typically thick, colorless to pale yellow syrups. For compatibility with automated RNA synthesis instrumentation, each of the products is dissolved in a pre-determined volume of anhydrous acetonitrile, and this solution is aliquoted into the appropriate number of serum vials to yield a 1.0-mmole quantity of phosphoramidite in each vial. The vials are then placed in a suitable vacuum desiccator and the solvent removed under high vacuum overnight. The atmosphere is then replaced with dry argon, the vials are capped with rubber septa, and the packaged phosphoramidites are stored at −20° C. until needed. Each phosphoramidite is dissolved in sufficient anhydrous acetonitrile to give the desired concentration prior to installation on the synthesis instrument.

The synthesis of the desired oligoribonucleotide is carried out using automated synthesis instrumentation. It begins with the 3′-terminal nucleotide covalently bound via its 3′-hydroxyl to a solid beaded polystyrene support through a cleavable linkage. The appropriate quantity of support for the desired synthesis scale is measured into a reaction cartridge, which is then affixed to synthesis instrument. The bound nucleoside is protected with a 5′-O-dimethoxytrityl moiety, which is removed with anhydrous acid (e.g., 3% [v/v] dichloroacetic acid in dichloromethane) in order to free the 5′-hydroxyl for chain assembly.

Subsequent nucleosides in the sequence to be assembled are sequentially added to the growing chain on the solid support using a four-step cycle, consisting of the following general reactions: coupling; oxidation; capping; and/or de-silylation. Coupling can be described when the appropriate phosphoramidite is activated with 5-ethylthio-1H-tetrazole and allowed to react with the free 5′-hydroxyl of the support bound nucleoside or oligonucleotide. This can also include optimization of the concentrations and molar excesses of these two reagents, as well as of the reaction time, results in coupling yields generally in excess of 98% per cycle.

Oxidation can be described when the internucleotide linkage is formed in the coupling step leaves the phosphorous atom in its P(III) [phosphite] oxidation state. The biologically-relevant oxidation state is P(V) [phosphate]. The phosphorous is therefore oxidized from P(III) to P(V) using a solution of tert-butylhydroperoxide in toluene.

Capping can be described when the small quantity of residual unreacted 5′-hydroxyl groups must be blocked from participation in subsequent coupling cycles in order to prevent the formation of deletion-containing sequences. This is accomplished by treating the support with a large excess of acetic anhydride and 1-methylimidazole in acetonitrile, which efficiently blocks residual 5′-hydroxyl groups as acetate esters.

De-silylation can be described when the silyl-protected 5′-hydroxyl must be deprotected prior to the next coupling reaction. This is accomplished through treatment with triethylamine trihydrogen fluoride in N,N-dimethylformamide, which rapidly and specifically liberates the 5′-hydroxyl without concomitant removal of other protecting groups (2′-O-ACE, N-acyl base-protecting groups, or phosphate methyl).

It should be noted that in between the above four reaction steps are several washes with acetonitrile, which are employed to remove the excess of reagents and solvents prior to the next reaction step. The above cycle is repeated the necessary number of times until the unmodified portion of the oligoribonucleotide has been assembled. The above synthesis method is only exemplary and should not be construed as limited the means by which the molecules may be made. Any method that is now known or that comes to be known for synthesizing siRNA and that from reading this disclosure one skilled in the art would conclude would be useful in connection with the present invention may be employed.

The shRNA and/or sfhRNAs of certain embodiments may include modified nucleosides (e.g., 2′-O-methyl derivatives). The 5′-O-silyl-2′-O-methyl-3′-O-phosphoramidite derivatives required for the introduction of these modified nucleosides are prepared using procedures similar to those described previously (e.g., steps 4 and 5 above), starting from base-protected 2′-O-methyl nucleosides (e.g., 2′-O-methyl-uridine, 2′-O-methyl-N-4-acetylcytidine, 2′-O-methyl-N2-isobutyrylguanosine and 2′-O-methyl-N-6-isobutyryladenosine). The absence of the 2′-hydroxyl in these modified nucleosides eliminates the need for ACE protection of these compounds. As such, introduction of the 5′-O-silyl and the reactive 3′-O-phosphoramidite moiety is accomplished in two steps, including: Protection of the 5′-hydroxy as a 5′-O-silyl ether using benzhydroxy-bis(trimethylsilyloxy)silyl chloride (e.g., BzH-Cl) in N,N-dimethylformamide, followed by chromatographic purification; and Conversion to the 3′ -O-phosphoramidite derivative using bis(N,N-diisopropylamino)methoxyphosphine and 5-ethylthio-1H-tetrazole in dichloromethane/acetonitrile, followed by chromatographic purification.

Post-purification packaging of the phosphoramidites is carried out using the procedures described previously for the standard nucleoside phosphoramidites. Similarly, the incorporation of the two 5′-O-silyl-2′-O-methyl nucleosides via their phosphoramidite derivatives is accomplished by twice applying the same four-step cycle described previously for the standard nucleoside phosphoramidites.

The shRNA and/or sfhRNAs of certain embodiments of this invention include a phosphate moiety at the 5′-end of a strand. This phosphate is introduced chemically as the final coupling to the antisense sequence. The required phosphoramidite derivative (e.g., bis(cyanoethyl)-N,N-diisopropylamino phosphoramidite) is synthesized as follows in brief: phosphorous trichloride is treated with one equivalent of N,N-diisopropylamine in anhydrous tetrahydrofuran in the presence of excess triethylamine. Then, two equivalents of 3-hydroxypropionitrile are added and allowed to react completely. Finally, the product is purified by chromatography. Post-purification packaging of the phosphoramidite is carried out using the procedures described previously for the standard nucleoside phosphoramidites. Similarly, the incorporation of the phosphoramidite at the 5′-end of the antisense strand is accomplished by applying the same four-step cycle described previously for the standard nucleoside phosphoramidites.

The modified, protected oligoribonucleotide remains linked to the solid support at the finish of chain assembly. A two-step rapid cleavage/deprotection procedure is used to remove the phosphate methyl protecting groups, cleave the oligoribonucleotide from the solid support, and remove the N-acyl base-protecting groups. It should be noted that this procedure also removes the cyanoethyl protecting groups from the 5′-phosphate on the antisense strand. Additionally, the procedure removes the acetyl functionalities from the ACE orthoester, converting the 2′-O-ACE protecting group into the bis(e.g., 2-hydroxyethyl)orthoester. This new orthoester is significantly more labile to mild acid, as well as more hydrophilic than the parent ACE group. The two-step procedure is briefly as follows the support-bound oligoribonucleotide is treated with a solution of disodium 2-carbamoyl-2-cyanoethylene-1,1-dithiolate trihydrate in N,N-dimethylformamide, where the reagent rapidly and efficiently removes the methyl protecting groups from the internucleotide phosphate linkages without cleaving the oligoribonucleotide from the solid support., and the support is then washed with water to remove excess dithiolate; and the oligoribonucleotide is cleaved from the solid support with 40% (w/v) aqueous methylamine at room temperature, where the methylamine solution containing the crude oligoribonucleotide is then heated to 55° C. to remove the protecting groups from the nucleoside bases, and the crude orthoester-protected oligoribonucleotide is obtained following solvent removal in vacuo.

Removal of the 2′-orthoesters is the final step in the synthesis process. This is accomplished by treating the crude oligoribonucleotide with an aqueous solution of acetic acid and N,N,N′,N′-tetramethyl ethylene diamine, pH 3.8, at 55° C. for 35 minutes. The completely deprotected oligoribonucleotide is then desalted by ethanol precipitation and isolated by centrifugation.

In addition, incorporation of fluorescent labels at the 5′-terminus of a polynucleotide is a common and well-understood manipulation for those skilled in the art. In general, there are two methods that are employed to accomplish this incorporation, and the necessary materials are available from several commercial sources (e.g., Glen Research Inc., Sterling, Va., USA; Molecular Probes Inc., Eugene, Oreg., USA; TriLink BioTechnologies Inc., San Diego, Calif., USA, and others). The first method utilizes a fluorescent molecule that has been derivatized with a phosphoramidite moiety similar to the phosphoramidite derivatives of the nucleosides described previously. In such case, the fluorescent dye is appended to the support-bound polynucleotide in the final cycle of chain assembly. The fluorophore-modified polynucleotide is then cleaved from the solid support and deprotected using the standard procedures described above. This method has been termed “direct labeling.” Alternatively, the second method utilizes a linker molecule derivatized with a phosphoramidite moiety that contains a protected reactive functional group (e.g., amino, sulfhydryl, carbonyl, carboxyl, and others). This linker molecule is appended to the support-bound polynucleotide in the final cycle of chain assembly. The linker-modified polynucleotide is then cleaved from the solid support and deprotected using the standard procedures described above. The functional group on the linker is deprotected either during the standard deprotection procedure, or by utilizing a subsequent group-specific treatment. The crude linker-modified polynucleotide is then reacted with an appropriate fluorophore derivative that will result in formation of a covalent bond between a site on the fluorophore and the functional group of the linker. This method has been termed “indirect labeling.”

Once synthesized, the polynucleotides of the present invention may immediately be used or be stored for future use. Preferably, the polynucleotides of the invention are stored in a suitable buffer. Many buffers are known in the art suitable for storing siRNAs and can be used for shRNA and/or sfhRNA. For example, the buffer may be comprised of 100 mM KCl, 30 mM HEPES-pH 7.5, and 1 mM Mg Cl₂. Preferably, the shRNA and/or sfhRNA of the present invention retain 30% to 100% of their activity when stored in such a buffer at 4° C. for one year. More preferably, they retain 80% to 100% of their biological activity when stored in such a buffer at 4° C. for one year. Alternatively, the compositions may be stored at −20° C. in such a buffer for at least a year or more. Preferably, storage for a year or more at −20° C. results in less than a 50% decrease in biological activity. More preferably, storage for a year or more at −20° C. results in less than a 20% decrease in biological activity after a year or more. Most preferably, storage for a year or more at −20° C. results in less than a 10% decrease in biological activity.

In order to ensure stability of the shRNA and/or sfhRNA prior to usage, they may be retained in dried-down form at −20° C. until they are ready for use. Prior to usage, they should be resuspended; however, once resuspended, for example, in the aforementioned buffer, they should be kept at −20° C. until used. The aforementioned buffer, prior to use, may be stored at approximately 4° C. or room temperature. In order to anneal shRNA and/or sfhRNA, deprotected RNA is diluted to 100 nM a standard Universal Buffer (e.g., 1× Universal Buffer composition is 20 mM KCl, 6 mM HEPES-KOH (pH 7.5), and 0.2 mM MgCl₂). Samples are then heated to 95° C. for five minutes, and then immediately transferred to an ice bath to ensure formation of the unimolecular molecule. In order to form fractured hairpins, equa-molar quantities of each strand are mixed. Samples are heated to 95° C. for one to five minutes, and then allowed to cool slowly to room temperature.

### Example 2

### Transfection

The transfections are performed according to the generalized protocol described below. A standardized transfection protocol in a 96 well can include the following: Protocols for all cells are fairly similar; Cells are plated at densities between 5,000 and 25,000 cells per well on the day before transfection; SuperRNAsin (Ambion) is added to transfection mixture for protection against RNAses; and All solutions and handling have to be carried out in RNAse free conditions.

The collection of cells can be described as follows: Add 2 ml of 0.05% trypsin-EDTA to a medium flask (e.g., T150, 50-70% confluent) or 6 ml to a large flask (e.g., T225), incubate 5 min at 37 degrees C.; Add 8 ml (e.g., T150) or 14 ml (e.g., T225) of regular media and pipette 10 times back and forth to re-suspend cells; Take 25 microliters of the cell suspension from step 2 and 75 microliters of trypan blue stain (e.g., 1:4) and place 10 microliters in a cell counter; Count number of cells in a standard hemocytometer; Average number of cells×4×10000 is number of cells per ml; Dilute with regular media to have 350,000/ml; Plate 5,000-25,000 cells per well in a 96 well plate; and Culture overnight.

A transfection protocol for 96 well plates can be described as follows: OPTI-MEM 2 ml+80 microliters Lipofectamine 2000 (e.g., 1:25)+15 microliters SuperRNAsin (AMBION); Transfer siRNA aliquots (e.g., 0.8 microliters of 100 micromolar to screen (e.g., total dilution factor is 1:750, 0.8 microliters of 100 micromolar solution will give 100 nanomolar final)) to the deep dish in a desired order (e.g., usually 3 columns×6 for 60 well format or four columns by 8 for 96 well); Transfer 100 microliters of OPTI-MEM; Transfer 100 microliters of OPTI-MEM with Lipofectamine 2000 and SuperRNAsin to each well; Leave for 20-30 min RT; Add 0.55 ml of regular media to each well; Cover plate with film and mix; and Array out 100×3×2 directly to the cells (e.g., sufficient for two plates). The mRNA or protein levels are measured 24, 48, 72, and/or 96 hours post transfection.

The level of siRNA-induced RNA interference, or gene silencing, was estimated by assaying the reduction in target mRNA levels or reduction in the corresponding protein levels. Assays of mRNA levels were carried out using B-DNA™ technology (Quantagene Corp.). Protein levels for fLUC and rLUC were assayed by STEADY GLO™ kits (Promega Corp.). Human alkaline phosphatase levels were assayed by Great EscAPe SEAP Fluorescence Detection Kits (#K2043-1), BD Biosciences, Clontech.

### Example 3

### Identifying a Preferred Loop Sequence and Performing a Walk miRNA Loop Design

In order to identify a preferred loop sequence, the sequences of 53 human miRNAs (see table below) were down-loaded from the miRNA data base (http://www.sanger.ac.uk/cgi-bin/Rfam/mirna/browse.pl). Sequences were then folded using MFOLD (http://www.bioinfo.rpi.edu/applications/mfold/old/rna/), and the number of nucleotides in each loop structure were determined. A tabulation of this data, including the name of the miRNA, and the loop size for each structure, are presented below in Table I.

TABLE I
Describing miRNA Sequences Utilized In This Study

| | | Loop | | | Loop |
|---|---|---|---|---|---|
| Number | Name | Size | Number | Name | Size |
| 1 | hsa-mir-101-1 | 12 | 27 | hsa-mir-139 | 11 |
| 2 | hsa-mir-101-2 | 9 | 28 | hsa-mir-142 | 17 |
| 3 | hsa-mir-103-1 | 12 | 29 | hsa-mir-145 | 14 |
| 4 | hsa-mir-103-2 | 7 | 30 | hsa-mir-15a | 13 |
| 5 | hsa-mir-107 | 5 | 31 | hsa-mir-15b | 7 |
| 6 | hsa-mir-1-1 | 5 | 32 | hsa-mir-16-1 | 9 |
| 7 | hsa-mir-1-2 | 14 | 33 | hsa-mir-17 | 7 |
| 8 | hsa-mir-141 | 17 | 34 | hsa-mir-16-2 | 17 |
| 9 | hsa-mir-143 | 6 | 35 | hsa-mir-18 | 16 |
| 10 | hsa-mir-147 | 20 | 36 | hsa-mir-186 | 14 |
| 11 | hsa-mir-148 | 7 | 37 | hsa-mir-194-1 | 13 |
| 12 | hsa-mir-17 | 7 | 38 | hsa-mir-196-1 | 18 |
| 13 | hsa-mir-197 | 19 | 39 | hsa-mir-198 | 14 |
| 14 | hsa-mir-199a-1 | 18 | 40 | hsa-mir-199a-1 | 20 |
| 15 | hsa-mir-199a-2 | 12 | 41 | hsa-mir-31 | 8 |
| 16 | hsa-mir-19a | 7 | 42 | hsa-mir-32 | 12 |
| 17 | hsa-mir-19b-1 | 13 | 43 | hsa-mir-33 | 13 |
| 18 | hsa-mir-19b-2 | 24 | 44 | hsa-mir-93 | 14 |
| 19 | hsa-mir-200b | 7 | 45 | hsa-mir-95 | 6 |
| 20 | hsa-mir-200c | 8 | 46 | hsa-mir-96 | 6 |
| 21 | hsa-mir-100 | 11 | 47 | hsa-mir-98 | 11 |
| 22 | hsa-mir-105-1 | 7 | 48 | hsa-mir-34a | 17 |
| 23 | hsa-mir-105-2 | 7 | 49 | hsa-mir-34b | 5 |
| 24 | hsa-mir-106a | 11 | 50 | hsa-mir-34c | 4 |
| 25 | hsa-mir-106b | 6 | 51 | hsa-mir-124a-1 | 13 |
| 26 | hsa-mir-129-2 | 10 | 52 | hsa-mir-124a-2 | 11 |
| 27 | hsa-mir-139 | 11 | 53 | hsa-mir-124a-3 | 12 |

Analysis of the loop structure of the 53 miRNA showed that two loop sizes, 7 nucleotides, and 13 nucleotides, were most prevalent. As the objective of this study was to choose a loop structure that made synthesis of larger hairpins more manageable, the 7 nucleotide loop was chosen for future design considerations. Furthermore, as an analysis of all available 7 nucleotide loop sequences from the collection failed to identify a consensus sequence or motif, the hsa-mir-17 sequence was chosen and tested by the inventors for hairpin design.

### Example 4

### Comparing siRNA and shRNA Over a Region of the DBI Gene

To understand the differences in functionality of 19 nt siRNA and shRNA having 31 bp stem regions and a loop (5′-AUAUGUG-3′, SEQ ID. NO. 1) derived from the hsa-mir-17 sequence, a “walk” covering 27 consecutive positions of a region of the DBI gene (Accession No.: NM_{—}020408, positions 220-249) was synthesized using 2′-ACE chemistry. shRNA were synthesized as both left-handed hairpins (e.g., 5′-AS-loop-S-3′) and right-handed hairpins (e.g., 5′-S-loop-AS-3′). Subsequently, each molecule was compared with the equivalent siRNA (i.e., siRNA having equivalent 5′ AS termini as left-handed hairpins by transfecting the molecules into HeLa cells at either 100 nM or 10 nM concentrations (Lipid=Dharmafect1; Dharmacon Inc., Lafayette, Colo.). Note: the cleavage product of right-handed hairpins has the same 5′ AS termini as the control siRNA for each position). Subsequently, the level of silencing of the DBI gene was assessed using a bDNA assay (Genospectra). The list of sequences used in this study are shown in Tables II-V below:

TABLE II

| | | |
|---|---|---|
| # | Name | Sequence of hairpins |

| | | |
|---|---|---|
| 1 | dbi19as_s | uuucagcucauuccaggcaucccacuuggccauaugugggccaagugggaugccuggaaugagcugaaauu |
| 2 | dbi20as_s | cuuucagcucauuccaggcaucccacuuggcauauguggccaagugggaugccuggaaugagcugaaaguu |
| 3 | dbi21as_s | ccuuucagcucauuccaggcaucccacuuggauaugugccaagugggaugccuggaaugagcugaaagguu |
| 4 | dbi22as_s | cccuuucagcucauuccaggcaucccacuugauaugugcaagugggaugccUggaaUgagcUgaaaggguu |
| S | dbi23as_s | ucccuuucagcucauuccaggcaucccacuuauaugugaagugggaugccUggaaUgagcUgaaagggauu |
| 6 | dbi24as_s | gucccuuucagcucauuccaggcaucccacuauaugugagugggaugccUggaaUgagcUgaaagggacuu |
| 7 | dbi25as_s | agucccuuucagcucauuccaggcaucccacauauguggugggaugccuggaaugagcugaaagggacuuu |
| 8 | dbi26as_s | aagucccuuucagcucauuccaggcaucccaauaugugugggaugccuggaaUgagcUgaaagggacUUuu |
| 9 | dbi27as_s | gaagucccuuucagcucauuccaggcaucccauauguggggaugccuggaaugagcugaaagggacuucuu |
| 10 | dbi28as_s | ggaagucccuuucagcucauuccaggcauccauaugugggaugccuggaaugagcugaaagggacuuccuu |
| 11 | dbi29as_s | uggaagucccuuucagcucauuccaggcaucauauguggaugccuggaaugagcugaaagggacuuccauu |
| 12 | dbi30as_s | uuggaagucccuuucagcucauuccaggcauauaugugaugccuggaaugagcugaaagggacuuccaauu |
| 13 | dbi31as_s | cuuggaagucccuuucagcucauuccaggcaauaugugugccuggaaugagcugaaagggacuuccaaguu |
| 14 | dbi32as_s | ccuuggaagucccuuucagcucauuccaggcauauguggccuggaaugagcugaaagggacuuccaagguu |
| 15 | dbi33as_s | uccuuggaagucccuuucagcucauuccaggauaugugccuggaaugagcugaaagggacuuccaaggauu |
| 16 | dbi34as_s | uuccuuggaagucccuuucagcucauuccagauaugugcuggaaugagcugaaagggacuuccaaggaauu |
| 17 | dbi35as_s | cuuccuuggaagucccuuucagcucauuccaauauguguggaaugagcugaaagggacuuccaaggaaguu |
| 18 | dbi36as_s | ucuuccuuggaagucccuuucagcucauuccauaugugggaaugagcugaaagggacuuccaaggaagauu |
| 19 | dbi37as_s | aucuuccuuggaagucccuuucagcucauucauauguggaaugagcugaaagggacuuccaaggaagauuu |
| 20 | dbi38as_s | caucuuccuuggaagucccuuucagcucauuauaugugaaugagcugaaagggacuuccaaggaagauguu |
| 21 | dbi39as_s | gcaucuuccuuggaagucccuuucagcucauauaugugaugagcugaaagggacuuccaaggaagaugcuu |
| 22 | dbi40as_s | ggcaucuuccuuggaagucccuuucagcucaauaugugugagcugaaagggacuuccaaggaagaugccuu |
| 23 | dbi41as_s | uggcaucuuccuuggaagucccuuucagcucauauguggagcugaaagggacuuccaaggaagaugccauu |
| 24 | dbi42as_s | auggcaucuuccuuggaagucccuuucagcuauaugugagcugaaagggacuuccaaggaagaugccauuu |
| 25 | dbi43as_s | cauggcaucuuccuuggaagucccuuucagcauauguggcugaaagggacuuccaaggaagaugccauguu |
| 26 | dbi44as_s | ucauggcaucuuccuuggaagucccuuucagauaugugcugaaagggacuuccaaggaagaugccaugauu |
| 27 | dbi45as_s | uucauggcaucuuccuuggaagucccuuucaauaugugugaaagggacuuccaaggaagaugccaugaauu |
| 28 | dbi46as_s | uuucauggcaucuuccuuggaagucccuuucauauguggaaagggacuuccaaggaagaugccaugaaauu |
| 29 | dbi47as_s | cuuucauggcaucuuccuuggaagucccuuuauaugugaaagggacuuccaaggaagaugccaugaaaguu |
| 30 | dbi48as_s | gcuuucauggcaucuuccuuggaagucccuuauaugugaagggacuuccaaggaagaugccaugaaagcuu |

TABLE III

| | | |
|---|---|---|
| 1 | dbi19s_as | gaugccuggaaugagcugaaagggacuuccaauauguguggaagucccuuucagcucauuccaggcaucuu |
| 2 | dbi20s_as | augccuggaaugagcugaaagggacuuccaaauauguguuggaagucccuuucagcucauuccaggcauuu |
| 3 | dbi21s_as | ugccuggaaugagcugaaagggacuuccaagauaugugcuuggaagucccuuucagcucauuccaggcauu |
| 4 | dbi22s_as | gccuggaaUgagcUgaaagggacuuccaaggauaugugccuuggaagucccuuucagcucauuccaggcuu |
| 5 | dbi23s_as | ccUggaaUgagcUgaaagggacuuccaaggaauauguguccuuggaagucccuuucagcucauuccagguu |
| 6 | dbi24s_as | cUggaaUgagcUgaaagggacuuccaaggaaauauguguuccuuggaagucccuuucagcucauuccaguu |
| 7 | dbi25s_as | uggaaugagcugaaagggacuuccaaggaagauaugugcuuccuuggaagucccuuucagcucauuccauu |
| 8 | dbi26s_as | ggaaUgagcUgaaagggacUUccaaggaagaauaugugucuuccuuggaagucccuuucagcucauuccuu |
| 9 | dbi27s_as | gaaugagcugaaagggacuuccaaggaagauauaugugaucuuccuuggaagucccuuucagcucauucuu |
| 10 | dbi28s_as | aaugagcugaaagggacuuccaaggaagaugauaugugcaucuuccuuggaagucccuuucagcucauuuu |
| 11 | dbi29s_as | augagcugaaagggacuuccaaggaagaugcauauguggcaucuuccuuggaagucccuuucagcucauuu |
| 12 | dbi30s_as | ugagcugaaagggacuuccaaggaagaugccauaugugggcaucuuccuuggaagucccuuucagcucauu |
| 13 | dbi31s_as | gagcugaaagggacuuccaaggaagaugccaauauguguggcaucuuccuuggaagucccuuucagcucuu |
| 14 | dbi32s_as | agcugaaagggacuuccaaggaagaugccauauaugugauggcaucuuccuuggaagucccuuucagcuuu |
| 15 | dbi33s_as | gcugaaagggacuuccaaggaagaugccaugauaugugcauggcaucuuccuuggaagucccuuucagcuu |
| 16 | dbi34s_as | cugaaagggacuuccaaggaagaugccaugaauaugugucauggcaucuuccuuggaagucccuuucaguu |
| 17 | dbi35s_as | ugaaagggacuuccaaggaagaugccaugaaauauguguucauggcaucuuccuuggaagucccuuucauu |
| 18 | dbi36s_as | gaaagggacuuccaaggaagaugccaugaaaauauguguuucauggcaucuuccuuggaagucccuuucuu |
| 19 | dbi37s_as | aaagggacuuccaaggaagaugccaugaaagauaugugcuuucauggcaucuuccuuggaagucccuuuuu |
| 20 | dbi38s_as | aagggacuuccaaggaagaugccaugaaagcauauguggcuuucauggcaucuuccuuggaagucccuuuu |
| 21 | dbi39s_as | agggacuuccaaggaagaugccaugaaagcuauaugugagcuuucauggcaucuuccuuggaagucccuuu |
| 22 | dbi40s_as | gggacuuccaaggaagaugccaugaaagcuuauaugugaagcuuucauggcaucuuccuuggaagucccuu |
| 23 | dbi41s_as | ggacuuccaaggaagaugccaugaaagcuuaauauguguaagcuuucauggcaucuuccuuggaaguccuu |
| 24 | dbi42s_as | gacuuccaaggaagaugccaugaaagcuuacauaugugguaagcuuucauggcaucuuccuuggaagucuu |
| 25 | dbi43s_as | acuuccaaggaagaugccaugaaagcuuacaauauguguguaagcuuucauggcaucuuccuuggaaguuu |
| 26 | dbi44s_as | cuuccaaggaagaugccaugaaagcuuacauauaugugauguaagcuuucauggcaucuuccuuggaaguu |
| 27 | dbi45s_as | uuccaaggaagaugccaugaaagcuuacaucauauguggauguaagcuuucauggcaucuuccuuggaauu |
| 28 | dbi46s_as | uccaaggaagaugccaugaaagcuuacaucaauaugugugauguaagcuuucauggcaucuuccuuggauu |
| 29 | dbi47s_as | ccaaggaagaugccaugaaagcuuacaucaaauauguguugauguaagcuuucauggcaucuuccuugguu |
| 30 | dbi48s_as | caaggaagaugccaugaaagcuuacaucaacauaugugguugauguaagcuuucauggcaucuuccuuguu |

TABLE IV

| siRNAs to | |
|---|---|
| DBI | |
| dbi19_S | gaugccuggaaugagcugauu |
| dbi20_S | augccuggaaugagcugaauu |
| dbi21_S | ugccuggaaugagcugaaauu |
| dbi22_S | gccuggaaugagcugaaaguu |
| dbi23_S | ccuggaaugagcugaaagguu |
| dbi24_S | cuggaaugagcugaaaggguu |
| dbi25_S | uggaaugagcugaaagggauu |
| dbi26_S | ggaaugagcugaaagggacuu |
| dbi27_S | gaaugagcugaaagggacuuu |
| dbi28_S | aaugagcugaaagggacuuuu |
| dbi29_S | augagcugaaagggacuucuu |
| dbi30_S | ugagcugaaagggacuuccuu |
| dbi31_S | gagcugaaagggacuuccauu |
| dbi32_S | agcugaaagggacuuccaauu |
| dbi33_S | gcugaaagggacuuccaaguu |
| dbi34_S | cugaaagggacuuccaagguu |
| dbi35_S | ugaaagggacuuccaaggauu |
| dbi36_S | gaaagggacuuccaaggaauu |
| dbi37_S | aaagggacuuccaaggaaguu |
| dbi38_S | aagggacuuccaaggaagauu |
| dbi39_S | agggacuuccaaggaagauuu |
| dbi40_S | gggacuuccaaggaagauguu |
| dbi41_S | ggacuuccaaggaagaugcuu |
| dbi42_S | gacuuccaaggaagaugccuu |
| dbi43_S | acuuccaaggaagaugccauu |
| dbi44_S | cuuccaaggaagaugccauuu |
| dbi45_S | uuccaaggaagaugccauguu |
| dbi46_S | uccaaggaagaugccaugauu |
| dbi47_S | ccaaggaagaugccaugaauu |
| dbi48_S | caaggaagaugccaugaaauu |
| dbi19_AS | ucagcucauuccaggcaucuu |
| dbi20_AS | uucagcucauuccaggcauuu |
| dbi21_AS | uuucagcucauuccaggcauu |
| dbi22_AS | cuuucagcucauuccaggcuu |
| dbi23_AS | ccuuucagcucauuccagguu |
| dbi24_AS | cccuuucagcucauuccaguu |
| dbi25_AS | ucccuuucagcucauuccauu |
| dbi26_AS | gucccuuucagcucauuccuu |
| dbi27_AS | agucccuuucagcucauucuu |
| dbi28_AS | aagucccuuucagcucauuuu |
| dbi29_AS | gaagucccuuucagcucauuu |
| dbi30_AS | ggaagucccuuucagcucauu |
| dbi31_AS | uggaagucccuuucagcucuu |
| dbi32_AS | uuggaagucccuuucagcuuu |
| dbi33_AS | cuuggaagucccuuucagcuu |
| dbi34_AS | ccuuggaagucccuuucaguu |
| dbi35_AS | uccuuggaagucccuuucauu |
| dbi36_AS | uuccuuggaagucccuuucuu |
| dbi37_AS | cuuccuuggaagucccuuuuu |
| dbi38_AS | ucuuccuuggaagucccuuuu |
| dbi39_AS | aucuuccuuggaagucccuuu |
| dbi40_AS | caucuuccuuggaagucccuu |
| dbi41_AS | gcaucuuccuuggaaguccuu |
| dbi42_AS | ggcaucuuccuuggaagucuu |
| dbi43_AS | uggcaucuuccuuggaaguuu |
| dbi44_AS | auggcaucuuccuuggaaguu |
| dbi45_AS | cauggcaucuuccuuggaauu |
| dbi46_AS | ucauggcaucuuccuuggauu |
| dbi47_AS | uucauggcaucuuccuugguu |
| dbi48_AS | uuucauggcaucuuccuuguu |

TABLE V

Sequences of Stems against DBI. Stem only
of the AS-loop-S hairpins (31 basepair
duplex with 2nt overhang on one end)

| | | |
|---|---|---|
| 1 | dbi19AS | uuucagcucauuccaggcaucccacuuggcc |
| 2 | dbi20AS | cuuucagcucauuccaggcaucccacuuggc |
| 3 | dbi21AS | ccuuucagcucauuccaggcaucccacuugg |
| 4 | dbi22AS | cccuuucagcucauuccaggcaucccacuug |
| 5 | dbi23AS | ucccuuucagcucauuccaggcaucccacuu |
| 6 | dbi24AS | gucccuuucagcucauuccaggcaucccacu |
| 7 | dbi25AS | agucccuuucagcucauuccaggcaucccac |
| 8 | dbi26AS | aagucccuuucagcucauuccaggcauccca |
| 9 | dbi27AS | gaagucccuuucagcucauuccaggcauccc |
| 10 | dbi28AS | ggaagucccuuucagcucauuccaggcaucc |
| 11 | dbi29AS | uggaagucccuuucagcucauuccaggcauc |
| 12 | dbi30AS | uuggaagucccuuucagcucauuccaggcau |
| 13 | dbi31AS | cuuggaagucccuuucagcucauuccaggca |
| 14 | dbi32AS | ccuuggaagucccuuucagcucauuccaggc |
| 15 | dbi33AS | uccuuggaagucccuuucagcucauuccagg |
| 16 | dbi34AS | uuccuuggaagucccuuucagcucauuccag |
| 17 | dbi35AS | cuuccuuggaagucccuuucagcucauucca |
| 18 | dbi36AS | ucuuccuuggaagucccuuucagcucauucc |
| 19 | dbi37AS | aucuuccuuggaagucccuuucagcucauuc |
| 20 | dbi38AS | caucuuccuuggaagucccuuucagcucauu |
| 21 | dbi39AS | gcaucuuccuuggaagucccuuucagcucau |
| 22 | dbi40AS | ggcaucuuccuuggaagucccuuucagcuca |
| 23 | dbi41AS | uggcaucuuccuuggaagucccuuucagcuc |
| 24 | dbi42AS | auggcaucuuccuuggaagucccuuucagcu |
| 25 | dbi43AS | cauggcaucuuccuuggaagucccuuucagc |
| 26 | dbi44AS | ucauggcaucuuccuuggaagucccuuucag |
| 27 | dbi45AS | uucauggcaucuuccuuggaagucccuuuca |
| 28 | dbi46AS | uuucauggcaucuuccuuggaagucccuuuc |
| 29 | dbi47AS | cuuucauggcaucuuccuuggaagucccuuu |
| 30 | dbi48AS | gcuuucauggcaucuuccuuggaagucccuu |
| 31 | dbi19S | ggccaagugggaugccuggaaugagcugaaauu |
| 32 | dbi20S | gccaagugggaugccuggaaugagcugaaaguu |
| 33 | dbi21S | ccaagugggaugccuggaaugagcugaaagguu |
| 34 | dbi22S | caagugggaugccuggaaugagcugaaaggguu |
| 35 | dbi23S | aagugggaugccuggaaugagcugaaagggauu |
| 36 | dbi24S | agugggaugccuggaaugagcugaaagggacuu |
| 37 | dbi25S | gugggaugccuggaaugagcugaaagggacuuu |
| 38 | dbi26S | ugggaugccuggaaugagcugaaagggacuuuu |
| 39 | dbi27S | gggaugccuggaaugagcugaaagggacuucuu |
| 40 | dbi28S | ggaugccuggaaugagcugaaagggacuuccuu |
| 41 | dbi29S | gaugccuggaaugagcugaaagggacuuccauu |
| 42 | dbi30S | augccuggaaugagcugaaagggacuuccaauu |
| 43 | dbi31S | ugccuggaaugagcugaaagggacuuccaaguu |
| 44 | dbi32S | gccuggaaugagcugaaagggacuuccaagguu |
| 45 | dbi33S | ccuggaaugagcugaaagggacuuccaaggauu |
| 46 | dbi34S | cuggaaugagcugaaagggacuuccaaggaauu |
| 47 | dbi35S | uggaaugagcugaaagggacuuccaaggaaguu |
| 48 | dbi36S | ggaaugagcugaaagggacuuccaaggaagauu |
| 49 | dbi37S | gaaugagcugaaagggacuuccaaggaagauuu |
| 50 | dbi38S | aaugagcugaaagggacuuccaaggaagauguu |
| 51 | dbi39S | augagcugaaagggacuuccaaggaagaugcuu |
| 52 | dbi40S | ugagcugaaagggacuuccaaggaagaugccuu |
| 53 | dbi41S | gagcugaaagggacuuccaaggaagaugccauu |
| 54 | dbi42S | agcugaaagggacuuccaaggaagaugccauuu |
| 55 | dbi43S | gcugaaagggacuuccaaggaagaugccauguu |
| 56 | dbi44S | cugaaagggacuuccaaggaagaugccaugauu |
| 57 | dbi45S | ugaaagggacuuccaaggaagaugccaugaauu |
| 58 | dbi46S | gaaagggacuuccaaggaagaugccaugaaauu |
| 59 | dbi47S | aaagggacuuccaaggaagaugccaugaaaguu |
| 60 | dbi48S | aagggacuuccaaggaagaugccaugaaagcuu |

Note:
“S” = sense, “AS” = antisense, “as_s” = antisense-loop-sense, “s_as” = sense-loop-antisense.

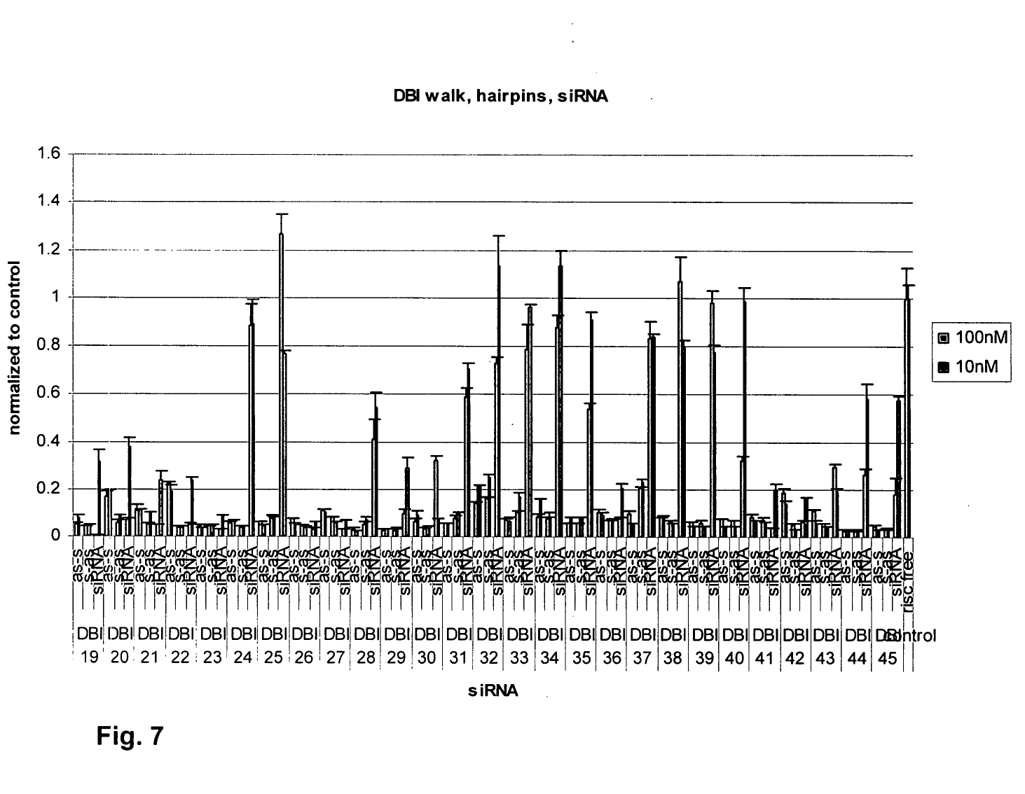

The results of these studies are shown in FIG. 7 and demonstrate the improved efficacy of the shRNA design of the invention. While the degree of silencing induced by 19 bp siRNA varies considerably over the region of the walk (see 100 nM concentration, roughly 0-20% silencing, DBI 24, 25, 32, 33, 34, 37, 38, 39; to >90% silencing for 100 nM concentrations for e.g., 20, 21, 22, and 23) the functionality of hairpins (both right-handed and left-handed molecules) designed along the guidelines of the invention consistently provided greater than 80% silencing. With few exceptions, the level of silencing produced by shRNA of the invention's design was consistent at both concentrations. This is in contrast to the performance of siRNAs at 100 and 10 nM concentrations (e.g., see DBI-20 siRNA, at 100 nM→90% silencing, at 10 nM→˜60% silencing). These result suggests that shRNAs of this design are more potent molecules.

### Example 5

### Performance of Hairpins Targeting Human Cyclophilins B and SEAP

To determine whether the superior performance of the hairpin of the invention was confined to a single gene (DBI, Example 3) or was applicable to silencing a wide range of genes, siRNA and shRNA targeting Human Cyclophilin B, and SEAP were designed and tested. The sequences used in these studies appear below in Tables VI-VII.

TABLE VI

| Cyclo and | |
|---|---|
| SEAP siRNAs | |
| cyclo195s | cggaaagacuguuccaaaauu |
| cyclo195as | uuuuggaacagucuuuccguu |
| cyclo247s | gagaaaggauuuggcuacauu |
| cyclo247as | uguagccaaauccuuucucuu |
| cyclo209s | caaaaacaguggauaauuuuu |
| cyclo209as | aaauuauccacuguuuuuguu |
| seap159s | gccaagaaccucaucaucuuu |
| seap159as | agaugaugagguucuuggcuu |
| seap1241s | cggaaacgguccaggcuauuu |
| seap1241as | auagccuggaccguuuccguu |
| seap806s | gaaccgcacugagcucauguu |
| seap806as | caugagcucagugcgguucuu |

TABLE VII

| | |
|---|---|
| Cyclo and SEAP | ShRNA |

| | |
|---|---|
| cyclo195as_s | guuuuuggaacagucuuuccgaagagaccaaauauguguuGGuCuCuucggaaagacuguuccaaaaacUU |
| cyclo195s_as | cggaaagacuguuccaaaaacAGuGGAuAAuauaugugauuauccacuguuuuuggaacagucuuuccgUU |
| cyclo247as_s | uuuguagccaaauccuuucucuccuguagcuauaugugAGCuACAGGAgagaaaggauuuggcuacaaaUU |
| cyclo247s_as | gagaaaggauuuggcuacaaaAACAGCAAAuauaugugauuugcuguuuuuguagccaaauccuuucucUU |
| cyclo209as_s | caaaauuauccacuguuuuuggaacagucuuauaugugAAGACuGuuCcaaaaacaguggauaauuuugUu |
| cyclo209s_as | caaaaacaguggauaauuuuguGGCCuuAGCauauguggcuaaggccacaaaauuauccacuguuuuugUU |
| seap159as_s | gaagaugaugagguucuuggcggcugucuguauaugugACAGACAGCCgccaagaaccucaucaucuucUU |
| seap159s_as | gccaagaaccucaucaucuucCuGGGCGAuGauaugugcaucgcccaggaagaugaugagguucuuggcUU |
| seap1241as_s | acauagccuggaccguuuccguauaggaggaauauguguCCuCCuAuAcggaaacgguccaggcuauguUU |
| seap1241s_as | cggaaacgguccaggcuauguGCuCAAGGACauaugugguccuugagcacauagccuggaccguuuccgUU |
| seapa806as_s | ugcaugagcucagugcgguuccacacauaccauaugugGGuAuGuGuGgaaccgcacugagcucaugcaUU |
| seapa806s_as | gaaccgcacugagcucaugcaGGCuuCCCuGauaugugcagggaagccugcaugagcucagugcgguucUU |

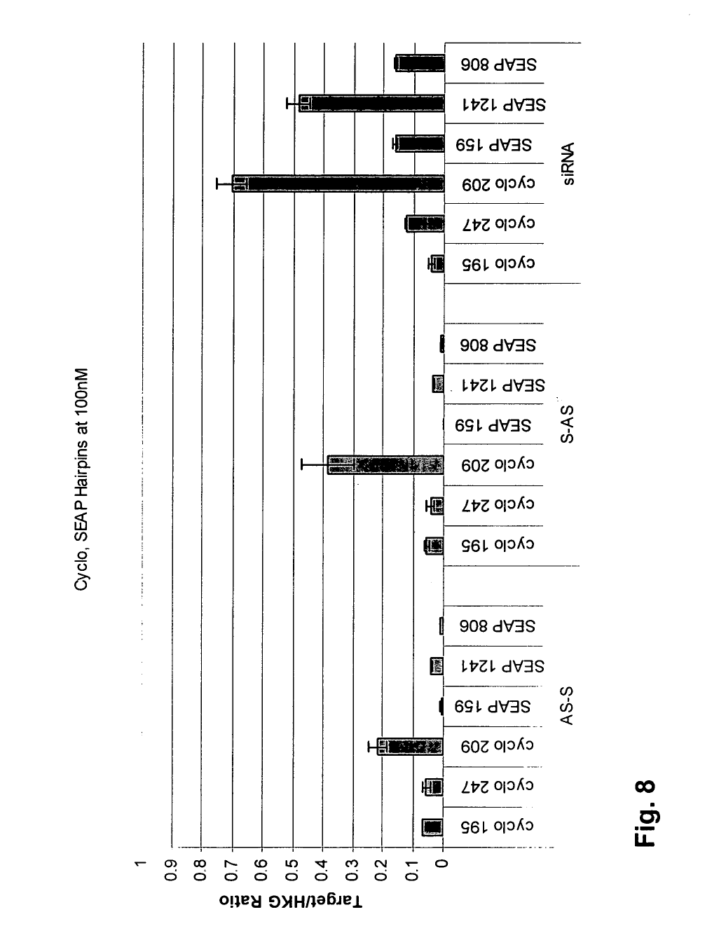

The data resulting from this study are presented in FIG. 8. For cyclophilin B, two of the three siRNA (e.g., targeting positions 195 and 247) induced 85% or better gene silencing and the third siRNA (e.g., targeting position 209) induced only 30% silencing. In comparison, the two shRNA targeting positions 195 and 247 performed equally, while the hairpin targeting position 209 induced ˜60% gene silencing (i.e., a 2× increase over the performance of the equivalent siRNA).

For SEAP, two of the three siRNA (e.g., targeting positions 159 and 806) induced approximately 85% silencing and the third siRNA (e.g., targeting position 1241) induced ˜50% silencing. In contrast, all shRNA targeting the SEAP gene performed exceptionally. Both right-handed and left-handed shRNA targeting all three positions (e.g., 159, 1241, and 806) induced greater than 95% silencing. These results strongly suggest that the functionality of the shRNA design of the invention is applicable to a wide range of genes.

### Example 6

### Performance of 31 mer siRNA

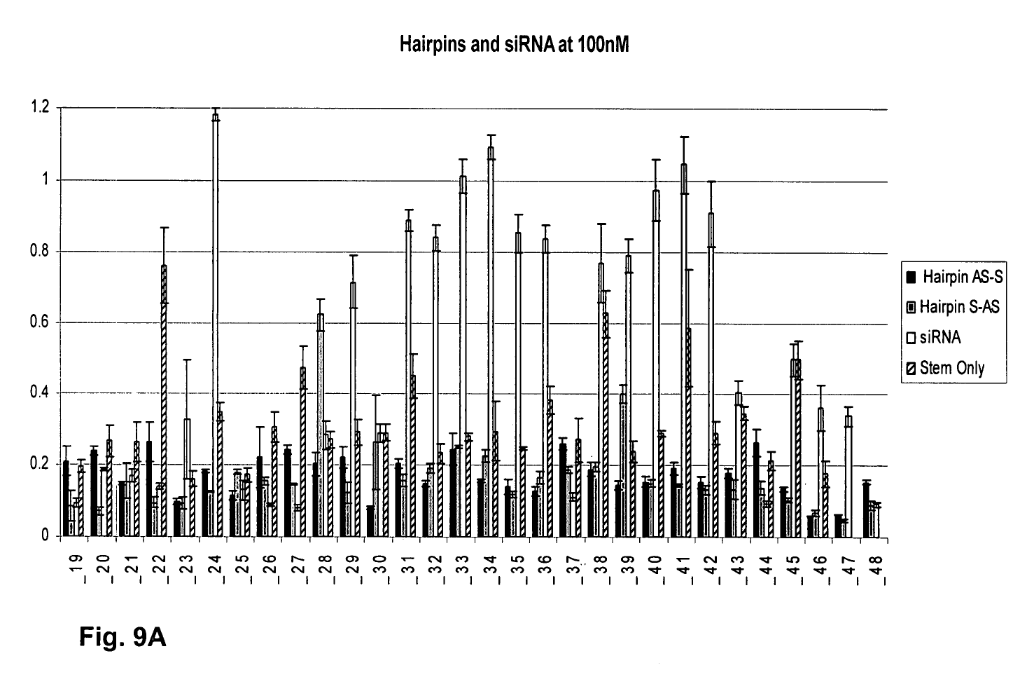

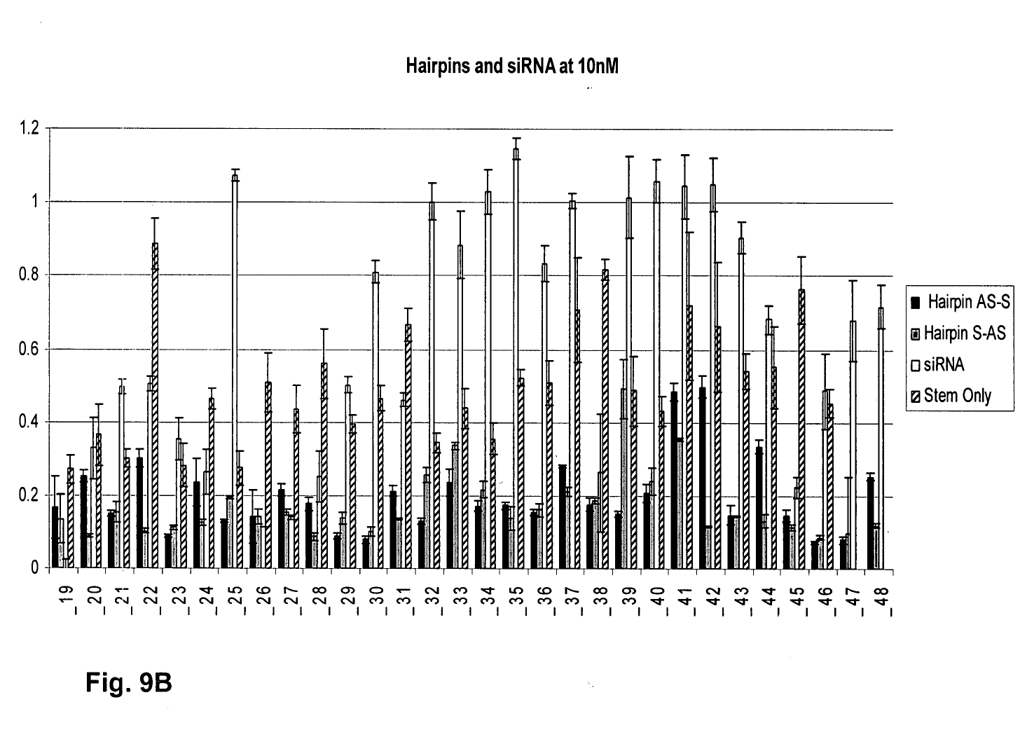

Recent reports in the literature have suggested that siRNA of 27 or more basepairs in length perform as well as equivalent hairpins (Kim, D. H. et al. (2004) Synthetic dsRNA Dicer substrates enhance RNAi potency and efficacy Nature Biotechnology, Advanced Online Publication). To test whether the improved performance observed with molecules of the invention was the result of the increased length of the duplex region (i.e., the stem) of the hairpin, the inventors compared the performance of shRNA having 31 bp stem sequences and a 5′-AUAUGUG-3′ loop (SEQ. ID NO. 1) derived from the hsa-mir-17 sequence, with 31 mer siRNA having the same sequence of the stem, at two concentrations (e.g., 100 nM and 10 nM). As shown in FIGS. 9A-9B, the functionality of hairpins and long 31 bp siRNA targeting the DBI walk are not equivalent, with hairpins having the design of the invention consistently inducing greater levels of silencing than the equivalent long duplexes (e.g., 31 bp stem). This is particularly obvious at the lower test concentrations (e.g., 10 nM). Thus, the high functionality of the hairpins of the invention are not simply the result of the increased length of the stem.

### Example 7

### Identifying Minimal Stem Length for Consistent Silencing Efficiency

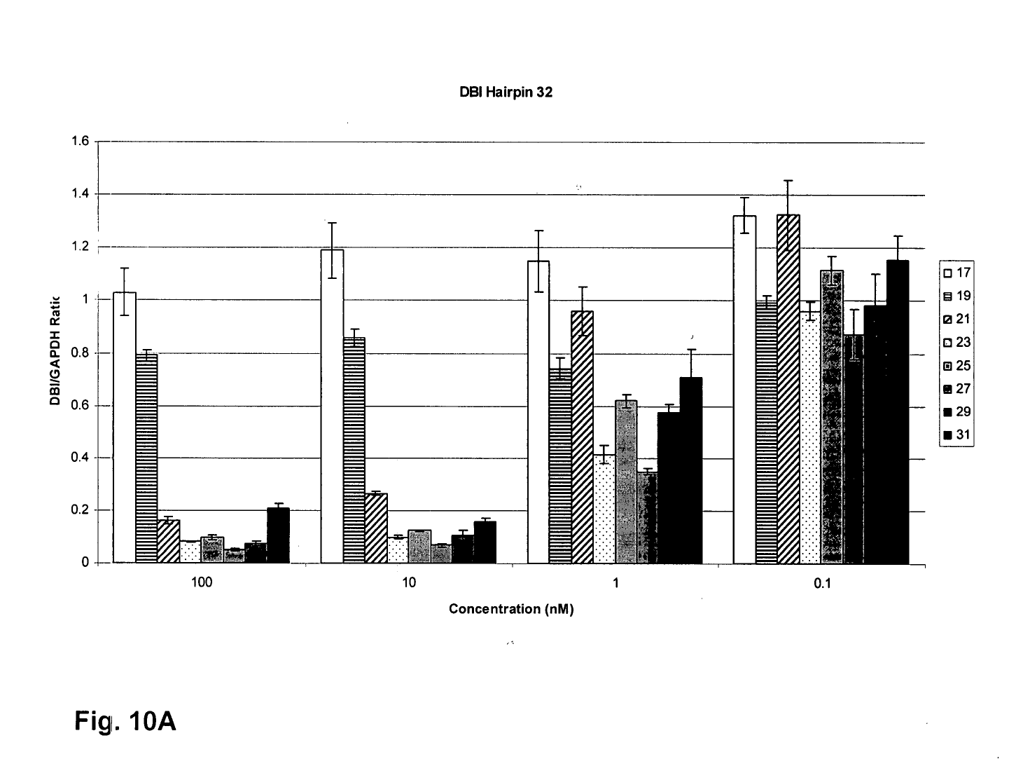

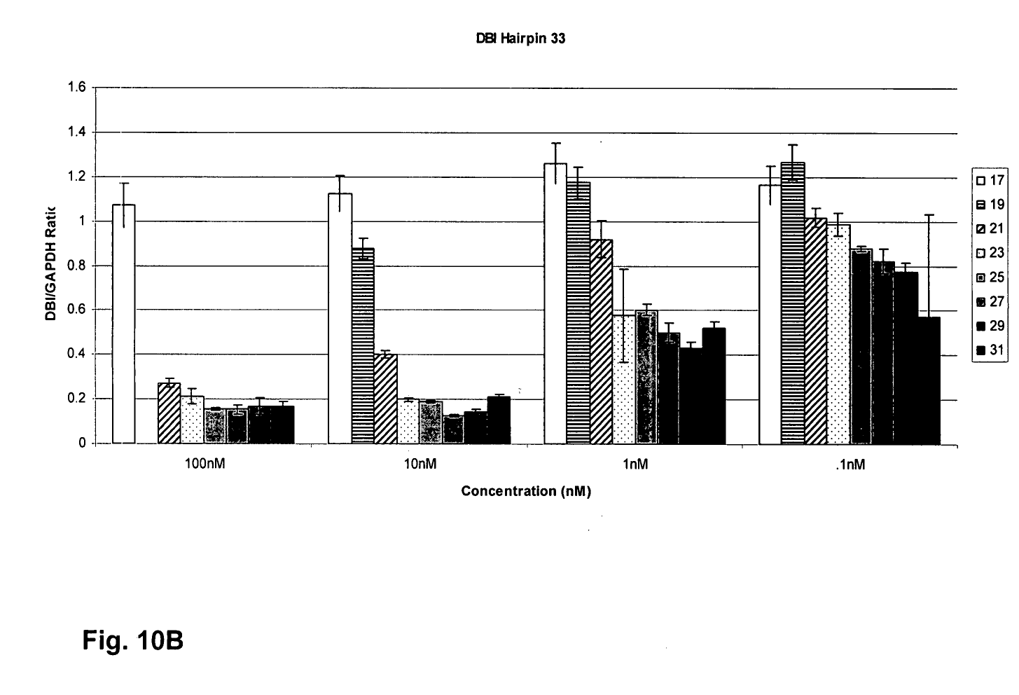

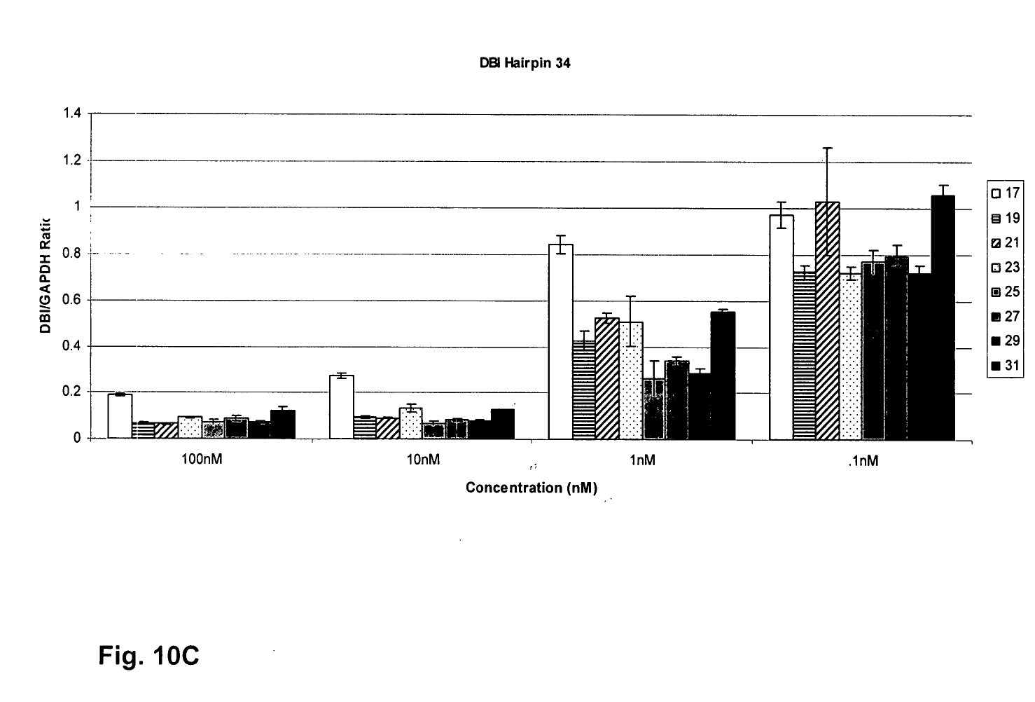

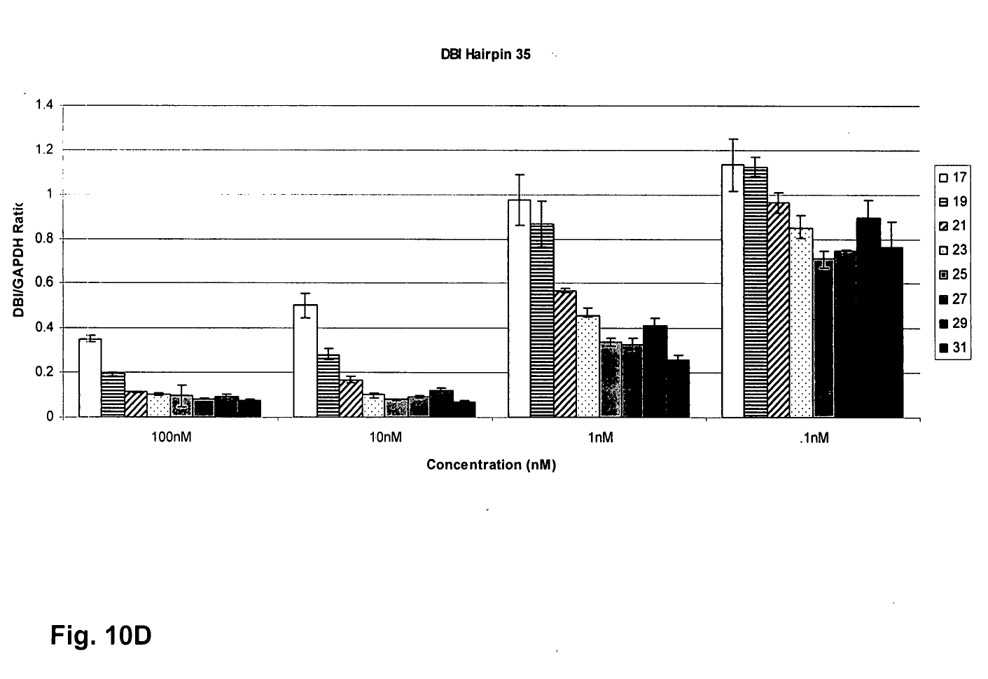

To determine what minimal stem length was necessary for hairpins of the invention to provide consistent silencing, four different hairpins targeting DBI (e.g., DBI 32, 33, 34, and 35) were tested with eight different stem lengths (31, 29, 27, 25, 23, 21, 19, and 17 bp) and four different concentrations (e.g., 100, 10, 1, and 0.1 nM). The results of these studies are presented in FIGS. 10A-10D and show the following: for DBI32, DBI33, and DBI 35, hairpins that had stems that were shorter than 26 bp in length silenced the intended target less efficiently than those that were 26 bp and longer. For DBI34, all hairpins that had stems that were 19 base pairs and longer performed similarly. Interestingly, in the case of DBI34, even the hairpin that had a 17 bp stem still functioned to silence gene expression by greater than 80% at 100 nM. This finding was somewhat surprising in that previous studies had suggested that siRNA shorter than 19 bp in length failed to enter RISC. The conclusions from these studies is that for consistent silencing, the stem length of the hairpin of the invention is preferably be 26 base pairs or longer.

### Example 8

### Performance of Fractured Hairpins Targeting DBI

To determine whether fractured hairpins performed similarly to shRNA, two hairpins targeting DBI (e.g., DBI25 and DBI34) were constructed in the configurations shown in FIGS. 10A-10B. Sequences used in these experiments are listed in the Table VII below. Note “S”=sense, “AS”=antisense, “//” indicates the position of the break relative to the sense, antisense, and loop structures, “shRNA”=short hairpin, and “f-shRNA”=fractured hairpin.

TABLE VII

| Target | Type of | Organiza- | Sequence(s) shown in |
|---|---|---|---|
| Site | Molecule | tion | 5′→-3′ orientation |
| DBI25 | shRNA | S-L-AS | UGGAAUGAGCUGAAAGGGACUUCCAA |
| | | | GGAAGAUAUGUGCUUCCUUGGAAGUC |
| | | | CCUUUCAGCUCAUUCCAUU |
| DBI25 | f-shRNA | S-//-L-AS | S: UGGAAUGAGCUGAAAGGGACUUC |
| | | | L-AS: CAAGGAAGAUAUGUGCUUCC |
| | | | UUGGAAGUCCCUUUCAGCUCAUUCCA |
| | | | UU |
| DBI25 | f-shRNA | S-L-//-AS | AS: AGUCCCUUUCAGCUCAUUCCA |
| | | | UU |
| | | | S-L: UGGAAUGAGCUGAAAGGGACU |
| | | | UCCAAGGAAGAUAUGUGCUUCCUUG |
| | | | GA |
| DBI25 | shRNA | AS-L-S | AGUCCCUUUCAGCUCAUUCCAGGCAU |
| | | | CCCACAUAUGUGGUGGGAUGCCUGGA |
| | | | AUGAGCUGAAAGGGACUUU |
| DBI25 | f-shRNA | AS-//-L-S | AS: AGUCCCUUUCAGCUCAUUCCA |
| | | | GG |
| | | | L-S: CAUCCCACAUAUGUGGUGGGA |
| | | | UGCCUGGAAUGAGCUGAAAGGGACU |
| | | | UU |
| DBI25 | f-shRNA | AS-L-//-S | S: UGGAAUGAGCUGAAAGGGACUUU |
| | | | AS-L: AGUCCCUUUCAGCUCAUUCC |
| | | | AGGCAUCCCACAUAUGUGGUGGGAUG |
| | | | GC |
| DBI34 | shRNA | S-L-AS | CUGAAAGGGACUUCCAAGGAAGAUGC |
| | | | CAUGAAUAUGUGUCAUGGCAUCUUCC |
| | | | UUGGAAGUCCCUUUCAGUU |
| DBI34 | f-shRNA | S-//-L-AS | S: CUGAAAGGGACUUCCAAGGAAGA |
| | | | L-AS: UGCCAUGAAUAUGUGUCAUG |
| | | | GCAUCUUCCUUGGAAGUCCCUUUCAG |
| | | | UU |
| DBI34 | f-shRNA | S-L-//-AS | AS:UUCCUUGGAAGUCCCUUUCAGUU |
| | | | S-L:CUGAAAGGGACUUCCAAGGAAG |
| | | | AUGCCAUGAAUAUGUGUCAUGGCAUC |
| DBI34 | shRNA | AS-L-S | UUCCUUGGAAGUCCCUUUCAGCUCAU |
| | | | UCCAGAUAUGUGCUGGAAUGAGCUGA |
| | | | AAGGGACUUCCAAGGAAUU |
| DBI34 | f-shRNA | AS-//-L-S | AS: UUCCUUGGAAGUCCCUUUCAG |
| | | | CU |
| | | | L-S: CAUUCCAGAUAUGUGCUGGAA |
| | | | UGAGCUGAAAGGGACUUCCAAGGAA |
| | | | UU |
| DBI34 | f-shRNA | AS-L-//-S | S: CUGAAAGGGACUUCCAAGGAAUU |
| | | | AS-L: UUCCUUGGAAGUCCCUUUCA |
| | | | GCUCAUUCCAGAUAUGUGCUGGAAUG |
| | | | AG |

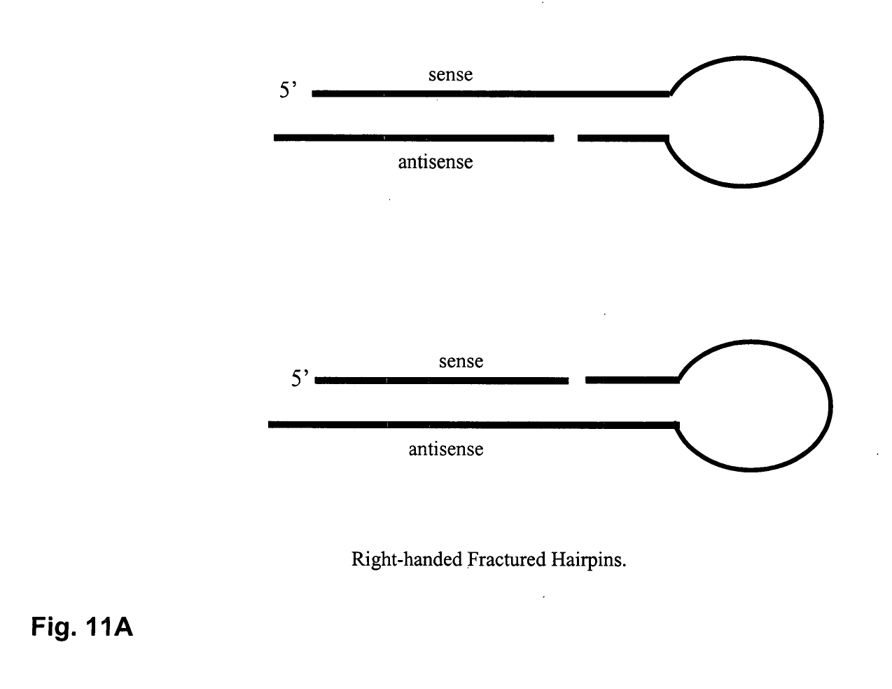

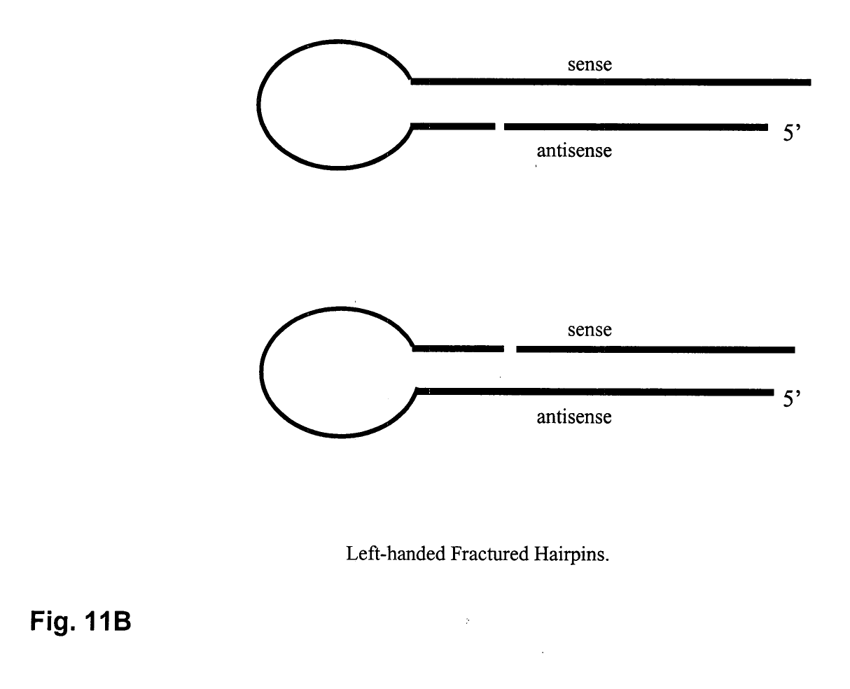

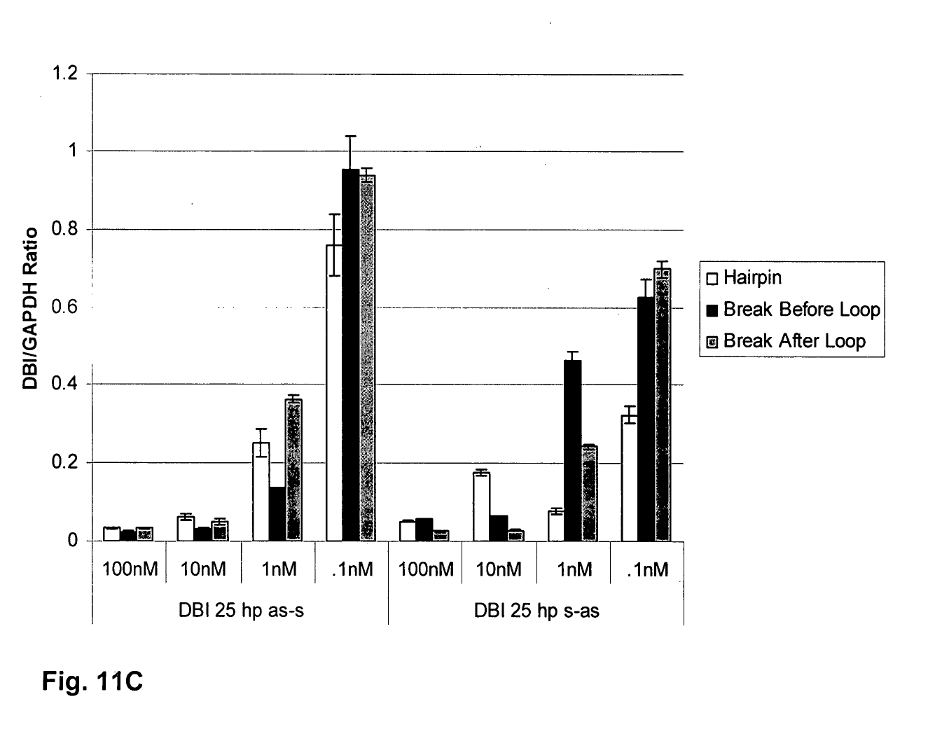

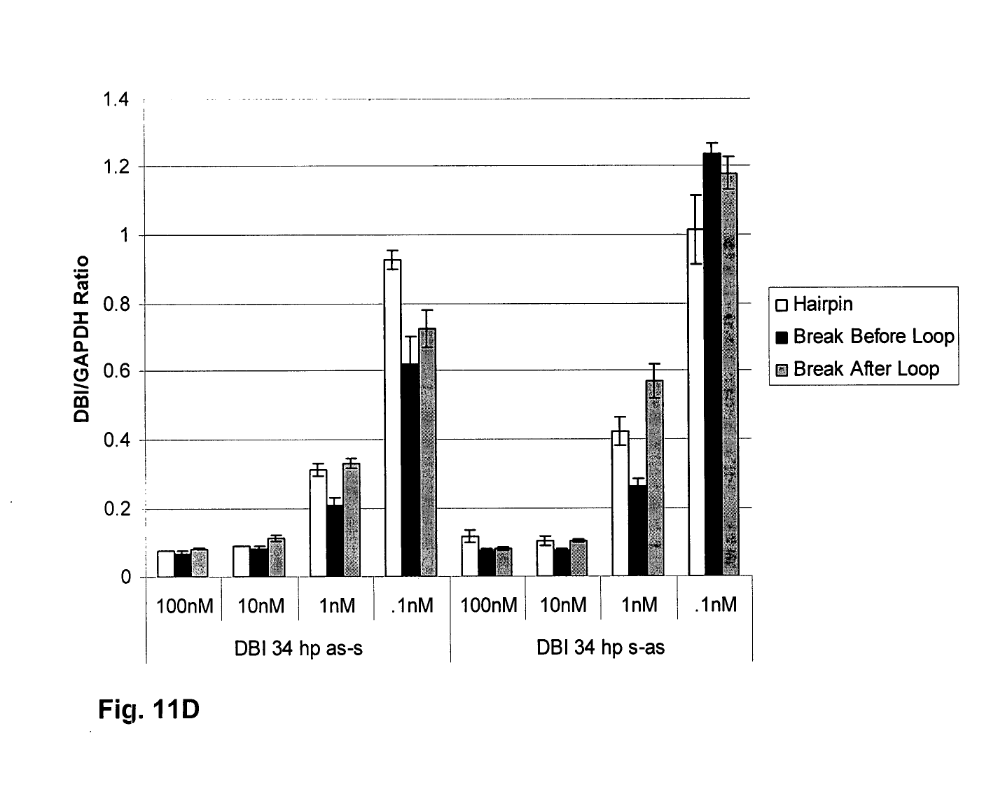

Synthetic shRNA and fractured shRNA were transfected into HeLa cells at four different concentrations (e.g., 100, 10, 1, and 0.1 nM) and tested for the ability to silence the DBI gene. The results of these studies are presented in FIGS. 11C-11D and 11d and show that the performance of fractured hairpins is similar to that of shRNA of the invention. At concentrations of 100 and 10 nM, both the shRNA and the fractured shRNA performed equivalently (e.g., >90% silencing). At lower concentrations, the level of DBI silencing was observed to drop off (<90%), but the relative degree of lost activity was (in most cases) roughly equivalent in all of the samples tested.

### Example 9

### Dicer Assays of Fractured Hairpins

To determine whether fractured shRNA gave Dicer digestion patterns that were equivalent to those obtained with e.g., equivalent dsRNA, an in vitro Dicer assay was performed on DBI25.

To accomplish this, RNAs were chemically synthesized using 2′ ACE chemistry and PAGE purified. RNA was then radioactively 5′ labeled using 32P-ATP (NEN) and polynucleotide kinase (Ambion) and purified by PAGE. Sequences corresponding to the Diazepam binding inhibitor (Accession number NM_{—}020548, DBI25) gene was used.

The Dicer assay was performed in 20 mM Tris-HCl pH7.5, 250 mM NaCl, 2.5 mM MgCl2. About 10 ul reactions were assembled using 0.05 units/ul human recombinant Dicer (Gene Therapy Systems or Stratagene or Invitrogen) and duplex RNA (containing 0.05-0.1 uM labeled strand RNA). Reactions were incubated for 0 to 3 hrs at 37° C. Reactions were stopped by adding 10 ul 80% Formamide/10 mM EDTA with 10 fold excess RNA complimentary to the unlabeled strand. After heating the samples for 5 min at 95° C., reactions were analyzed on 15% polyacrylamide/7M Urea gels. Gels were run for 3.5 hrs at 35 Watts and data were collected using the Storm PhosphorImager 860, and quantified and analyzed using Imagequant 5.2 (Molecular Dynamics).

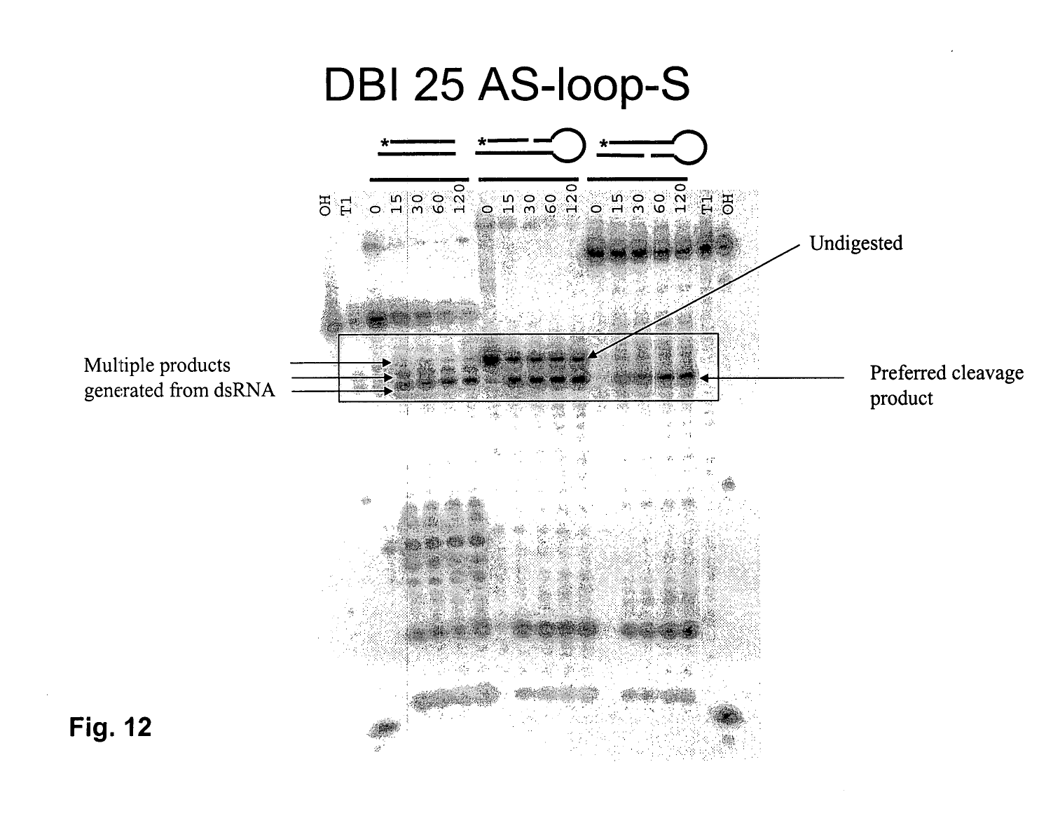

Previous studies have shown that the cleavage pattern generated by Dicer entering equivalent termini of dsRNA or shRNA having the same sequence, is comparable (see Vermeulen. A, et al. (2005) RNA 11(5)). To test whether this pattern is altered when fractured hairpins are used, the pattern generated by the fracture DBI25 hairpins (e.g., AS-//-loop-S, AS-loop-//-S) were compared with that of dsRNA having an equivalent sequence (see sequences above). The results of these studies are presented in FIG. 12. As shown in the boxed area, when Dicer enters dsRNA from the left hand side of the duplex, three distinct bands are generated, which demonstrates that while Dicer prefers the closest cleavage site, alternate sites can be utilized. Dicer digestion of the AS-//-loop-S fractured hairpin molecule generates a very different cleavage pattern. In this experiment, the smallest band is clearly the preferred substrate (e.g., the top band represents undigested material). Moreover, the efficiency at which the lower band is produced from this construct is a minimum of 2× greater than that observed in dsRNA, suggesting that fractured hairpins: (1) serve as more efficient substrates; and (2) produce a more limited (cleaner) set of products. A portion of these results are reiterated in the third substrate (e.g., AS-loop-//-S) where, again, the predominant band is the smallest band, and other alternative products are, for the most part, absent. Interestingly, the relative rate at which the product is generated using the AS-loop-//-S substrate is reduced compared to the AS-//-loop-S fractured hairpin, possibly suggesting that the two RNase domains of Dicer are not equivalent in their ability to cleave substrates. The results of these studies provide evidence that the use of fractured hairpins can be used to enhance Dicer cleavage and specificity.

What is claimed is:

1. A short hairpin polynucleotide for use in gene silencing, the polynucleotide comprising:
a polynucleotide having from about 42 nucleotides to about 106 nucleotides and being configured for being processed by Dicer, the polynucleotide comprising:
a first region having from about 19 to about 35 nucleotides;
a loop region coupled to the first region, the loop region having from about 4 to about 30 nucleotides;
a second region having from about 19 to about 35 nucleotides and having at least about 80% complementarity to the first region; and
optionally, an overhang region on one of the first region or second region and having less than about 6 nucleotides.

2. A polynucleotide as in claim 1, wherein the polynucleotide is comprised of about 71 nucleotides.

3. A polynucleotide as in claim 2, wherein the polynucleotide is comprised of at least one of the following:
the first region having about 31 nucleotides;
the loop region having about 7 nucleotides;
the second region having about 31 nucleotides; or
the overhang region having 2 nucleotides.

4. A polynucleotide as in claim 1, wherein the loop region comprises nucleotides having the sequence of SEQ. ID. NO. 1.

5. A polynucleotide as in claim 1, further comprising at least one of the following:
a sense region having a first 5′ sense nucleotide and a second 5′ sense nucleotide, the first and second 5′ sense nucleotides having a 2′ modification;
an antisense region having a first 5′ antisense nucleotide and a second 5′ antisense nucleotide, the first and second 5′ antisense nucleotides having a 2′ modification;
an antisense region having no antisense nucleotides with a 2′ modification; or
an antisense region having a second 5′ antisense nucleotide with a 2′ modification.

6. A polynucleotide as in claim 5, wherein the 2′ modification is a 2′-O-alkyl modification.

7. A polynucleotide as in claim 6, wherein the 2′-O-alkyl modification is a 2′-O-methyl modification.

8. A polynucleotide as in claim 5, wherein the polynucleotide is processed into a sense strand and an antisense strand by Dicer to obtain at least one of the following:
a sense strand having a first 5′ sense nucleotide at a first terminal nucleotide position and a second 5′ sense nucleotide at a second nucleotide position adjacent to the terminal nucleotide position, the first and second 5′ sense nucleotides having a 2′-O-alkyl modification;
an antisense strand having a first 5′ antisense nucleotide at a first terminal nucleotide position and a second 5′ antisense nucleotide at a second nucleotide position adjacent to the terminal nucleotide position, the first and second 5′ antisense nucleotides having a 2′-O-alkyl modification;
an antisense strand having no antisense nucleotides with a 2′ modification; or
an antisense strand having a first 5′ antisense nucleotide at a first terminal nucleotide position and a second 5′ antisense nucleotide at a second nucleotide position adjacent to the terminal nucleotide position, the second 5′ antisense nucleotide with a 2′-O-alkyl modification.

9. A polynucleotide as in claim 8, wherein the first 5′ antisense nucleotide includes a 5′ phosphate group.

10. A fractured hairpin for use in gene silencing, the hairpin comprising:
a first polynucleotide strand; and
a second polynucleotide strand capable of forming a hairpin structure with the first polynucleotide that is capable of being processed by Dicer, the hairpin structure having from about 42 to about 106 nucleotides, the second polynucleotide strand comprising:
a first region having at least 80% complementarity with the first strand and being capable of forming a first duplex region with the first strand;
a second region coupled to the first region;
a third region coupled to the second region; and
a fourth region coupled to the third region and having at least 80% complementarity with the second region, the fourth region being capable of forming a second duplex region with the second region such that the third region forms a loop adjacent to the second duplex region.

11. A hairpin as in claim 10, further comprising an overhang region having less than about 6 nucleotides on one of the first polynucleotide strand or first region of the second polynucleotide strand.

12. A hairpin as in claim 10, wherein the third region comprises nucleotides having the sequence of SEQ. ID. NO. 1.

13. A hairpin as in claim 10, wherein the fractured hairpin is a right-handed fractured hairpin by the first strand being an antisense strand and the first region of the second strand being a sense region.

14. A hairpin as in claim 10, wherein the fractured hairpin is a right-handed fractured hairpin by the first strand being a sense strand and the first region of the second strand being an antisense region.

15. A hairpin as in claim 10, wherein the fractured hairpin is a left-handed fractured hairpin by the first strand being an antisense strand and the first region of the second strand being a sense region.

16. A hairpin as in claim 10, wherein the fractured hairpin is a left-handed fractured hairpin by the first strand being a sense strand and the first region of the second strand being an antisense region.

17. A hairpin as in claim 10, wherein the first polynucleotide strand and second polynucleotide strand are processed into a sense strand and an antisense strand by Dicer to obtain at least one of the following:
a sense strand having a first 5′ sense nucleotide at a first terminal nucleotide position and a second 5′ sense nucleotide at a second nucleotide position adjacent to the terminal nucleotide position, the first and second 5′ sense nucleotides having a 2′-O-alkyl modification;
an antisense strand having a first 5′ antisense nucleotide at a first terminal nucleotide position and a second 5′ antisense nucleotide at a second nucleotide position adjacent to the terminal nucleotide position, the first and second 5′ antisense nucleotides having a 2′-O-alkyl modification;
an antisense strand having no antisense nucleotides with a 2′ modification; or
an antisense strand having a first 5′ antisense nucleotide at a first terminal nucleotide position and a second 5′ antisense nucleotide at a second nucleotide position adjacent to the terminal nucleotide position, the second 5′ antisense nucleotide with a 2′-O-alkyl modification.

18. A short hairpin RNA for use in gene silencing, the short hairpin comprising:
at least one polynucleotide forming the short hairpin RNA having from about 42 nucleotides to about 106 nucleotides and being configured for being processed by Dicer, the hairpin RNA comprising:
a first region;
a loop region coupled to the first region; and
a second region coupled to the loop region and being capable of forming a first duplex region with the first region;
wherein at least one of the first or second regions includes at least two tandem nucleotides each having a 2′ modification such that processing by Dicer results in a sense strand having a first 5′ sense nucleotide at a first terminal nucleotide position and a second 5′ sense nucleotide at a second nucleotide position adjacent to the terminal nucleotide position, the first and second 5′ sense nucleotides having the 2′-modification.

19. A hairpin RNA as in claim 18, wherein the other of the first or second region includes a nucleotide having a 2′ modification such that processing by Dicer results in an antisense strand having a first 5′ antisense nucleotide at a first terminal nucleotide position and a second 5′ antisense nucleotide at a second nucleotide position adjacent to the terminal nucleotide position, the second 5′ antisense nucleotide having the 2′-modification.

20. A hairpin RNA as in claim 18, wherein the other of the first or second region includes a nucleotide having a 2′ modification such that processing by Dicer results in an antisense strand having a first 5′ antisense nucleotide at a first terminal nucleotide position and a second 5′ antisense nucleotide at a second nucleotide position adjacent to the terminal nucleotide position, the first and second 5′ antisense nucleotides having the 2′-modification.

21. A hairpin RNA as in claim 18, wherein the 2′ modification is a 2′-O-alkyl modification.

22. A hairpin RNA as in claim 21, wherein the 2′-O-alkyl modification is a 2′-O-methyl modification.

23. A hairpin RNA as in claim 19, wherein processing by Dicer results in an antisense strand substantially devoid of nucleotides having a 2′ modification.

24. A hairpin RNA as in claim 18, wherein the hairpin RNA is comprised of about 71 nucleotides.

25. A polynucleotide as in claim 24, wherein the hairpin RNA is comprised of at least one of the following:
the first region having about 31 nucleotides;
the loop region having about 7 nucleotides;
the second region having about 31 nucleotides; or
an overhang region having 2 nucleotides.

26. A polynucleotide as in claim 18, wherein the loop region comprises nucleotides having the sequence of SEQ. ID. NO. 1.


SEQUENCE LISTING (205 sequences)

SEQ ID NO: 1 - RNA, 7 nt, Homo sapiens
auaugug

SEQ ID NO: 2 - RNA, 71 nt, Homo sapiens
uuucagcucauuccaggcaucccacuuggccauaugugggccaagugggaugccuggaaugagcugaaauu

SEQ ID NO: 3 - RNA, 71 nt, Homo sapiens
cuuucagcucauuccaggcaucccacuuggcauauguggccaagugggaugccuggaaugagcugaaaguu

SEQ ID NO: 4 - RNA, 71 nt, Homo sapiens
ccuuucagcucauuccaggcaucccacuuggauaugugccaagugggaugccuggaaugagcugaaagguu

SEQ ID NO: 5 - RNA, 71 nt, Homo sapiens
cccuuucagcucauuccaggcaucccacuugauaugugcaagugggaugccuggaaugagcugaaaggguu

SEQ ID NO: 6 - RNA, 71 nt, Homo sapiens
ucccuuucagcucauuccaggcaucccacuuauaugugaagugggaugccuggaaugagcugaaagggauu

SEQ ID NO: 7 - RNA, 71 nt, Homo sapiens
gucccuuucagcucauuccaggcaucccacuauaugugagugggaugccuggaaugagcugaaagggacuu

SEQ ID NO: 8 - RNA, 71 nt, Homo sapiens
agucccuuucagcucauuccaggcaucccacauauguggugggaugccuggaaugagcugaaagggacuuu

SEQ ID NO: 9 - RNA, 71 nt, Homo sapiens
aagucccuuucagcucauuccaggcaucccaauaugugugggaugccuggaaugagcugaaagggacuuuu

SEQ ID NO: 10 - RNA, 71 nt, Homo sapiens
gaagucccuuucagcucauuccaggcaucccauauguggggaugccuggaaugagcugaaagggacuucuu

SEQ ID NO: 11 - RNA, 71 nt, Homo sapiens
ggaagucccuuucagcucauuccaggcauccauaugugggaugccuggaaugagcugaaagggacuuccuu

SEQ ID NO: 12 - RNA, 71 nt, Homo sapiens
uggaagucccuuucagcucauuccaggcaucauauguggaugccuggaaugagcugaaagggacuuccauu

SEQ ID NO: 13 - RNA, 71 nt, Homo sapiens
uuggaagucccuuucagcucauuccaggcauauaugugaugccuggaaugagcugaaagggacuuccaauu

SEQ ID NO: 14 - RNA, 71 nt, Homo sapiens
cuuggaagucccuuucagcucauuccaggcaauaugugugccuggaaugagcugaaagggacuuccaaguu

SEQ ID NO: 15 - RNA, 71 nt, Homo sapiens
ccuuggaagucccuuucagcucauuccaggcauauguggccuggaaugagcugaaagggacuuccaagguu

SEQ ID NO: 16 - RNA, 71 nt, Homo sapiens
uccuuggaagucccuuucagcucauuccaggauaugugccuggaaugagcugaaagggacuuccaaggauu

SEQ ID NO: 17 - RNA, 71 nt, Homo sapiens
uuccuuggaagucccuuucagcucauuccagauaugugcuggaaugagcugaaagggacuuccaaggaauu

SEQ ID NO: 18 - RNA, 71 nt, Homo sapiens
cuuccuuggaagucccuuucagcucauuccaauauguguggaaugagcugaaagggacuuccaaggaaguu

SEQ ID NO: 19 - RNA, 71 nt, Homo sapiens
ucuuccuuggaagucccuuucagcucauuccauaugugggaaugagcugaaagggacuuccaaggaagauu

SEQ ID NO: 20 - RNA, 71 nt, Homo sapiens
aucuuccuuggaagucccuuucagcucauucauauguggaaugagcugaaagggacuuccaaggaagauuu

SEQ ID NO: 21 - RNA, 71 nt, Homo sapiens
caucuuccuuggaagucccuuucagcucauuauaugugaaugagcugaaagggacuuccaaggaagauguu

SEQ ID NO: 22 - RNA, 71 nt, Homo sapiens
gcaucuuccuuggaagucccuuucagcucauauaugugaugagcugaaagggacuuccaaggaagaugcuu

SEQ ID NO: 23 - RNA, 71 nt, Homo sapiens
ggcaucuuccuuggaagucccuuucagcucaauaugugugagcugaaagggacuuccaaggaagaugccuu

SEQ ID NO: 24 - RNA, 71 nt, Homo sapiens
uggcaucuuccuuggaagucccuuucagcucauauguggagcugaaagggacuuccaaggaagaugccauu

SEQ ID NO: 25 - RNA, 71 nt, Homo sapiens
auggcaucuuccuuggaagucccuuucagcuauaugugagcugaaagggacuuccaaggaagaugccauuu

SEQ ID NO: 26 - RNA, 71 nt, Homo sapiens
cauggcaucuuccuuggaagucccuuucagcauauguggcugaaagggacuuccaaggaagaugccauguu

SEQ ID NO: 27 - RNA, 71 nt, Homo sapiens
ucauggcaucuuccuuggaagucccuuucagauaugugcugaaagggacuuccaaggaagaugccaugauu

SEQ ID NO: 28 - RNA, 71 nt, Homo sapiens
uucauggcaucuuccuuggaagucccuuucaauaugugugaaagggacuuccaaggaagaugccaugaauu

SEQ ID NO: 29 - RNA, 71 nt, Homo sapiens
uuucauggcaucuuccuuggaagucccuuucauauguggaaagggacuuccaaggaagaugccaugaaauu

SEQ ID NO: 30 - RNA, 71 nt, Homo sapiens
cuuucauggcaucuuccuuggaagucccuuuauaugugaaagggacuuccaaggaagaugccaugaaaguu

SEQ ID NO: 31 - RNA, 71 nt, Homo sapiens
gcuuucauggcaucuuccuuggaagucccuuauaugugaagggacuuccaaggaagaugccaugaaagcuu

SEQ ID NO: 32 - RNA, 71 nt, Homo sapiens
gaugccuggaaugagcugaaagggacuuccaauauguguggaagucccuuucagcucauuccaggcaucuu

SEQ ID NO: 33 - RNA, 71 nt, Homo sapiens
augccuggaaugagcugaaagggacuuccaaauauguguuggaagucccuuucagcucauuccaggcauuu

SEQ ID NO: 34 - RNA, 71 nt, Homo sapiens
ugccuggaaugagcugaaagggacuuccaagauaugugcuuggaagucccuuucagcucauuccaggcauu

SEQ ID NO: 35 - RNA, 71 nt, Homo sapiens
gccuggaaugagcugaaagggacuuccaaggauaugugccuuggaagucccuuucagcucauuccaggcuu

SEQ ID NO: 36 - RNA, 71 nt, Homo sapiens
ccuggaaugagcugaaagggacuuccaaggaauauguguccuuggaagucccuuucagcucauuccagguu

SEQ ID NO: 37 - RNA, 71 nt, Homo sapiens
cuggaaugagcugaaagggacuuccaaggaaauauguguuccuuggaagucccuuucagcucauuccaguu

SEQ ID NO: 38 - RNA, 71 nt, Homo sapiens
uggaaugagcugaaagggacuuccaaggaagauaugugcuuccuuggaagucccuuucagcucauuccauu

SEQ ID NO: 39 - RNA, 71 nt, Homo sapiens
ggaaugagcugaaagggacuuccaaggaagaauaugugucuuccuuggaagucccuuucagcucauuccuu

SEQ ID NO: 40 - RNA, 71 nt, Homo sapiens
gaaugagcugaaagggacuuccaaggaagauauaugugaucuuccuuggaagucccuuucagcucauucuu

SEQ ID NO: 41 - RNA, 71 nt, Homo sapiens
aaugagcugaaagggacuuccaaggaagaugauaugugcaucuuccuuggaagucccuuucagcucauuuu

SEQ ID NO: 42 - RNA, 71 nt, Homo sapiens
augagcugaaagggacuuccaaggaagaugcauauguggcaucuuccuuggaagucccuuucagcucauuu

SEQ ID NO: 43 - RNA, 71 nt, Homo sapiens
ugagcugaaagggacuuccaaggaagaugccauaugugggcaucuuccuuggaagucccuuucagcucauu

SEQ ID NO: 44 - RNA, 71 nt, Homo sapiens
gagcugaaagggacuuccaaggaagaugccaauauguguggcaucuuccuuggaagucccuuucagcucuu

SEQ ID NO: 45 - RNA, 71 nt, Homo sapiens
agcugaaagggacuuccaaggaagaugccauauaugugauggcaucuuccuuggaagucccuuucagcuuu

SEQ ID NO: 46 - RNA, 71 nt, Homo sapiens
gcugaaagggacuuccaaggaagaugccaugauaugugcauggcaucuuccuuggaagucccuuucagcuu

SEQ ID NO: 47 - RNA, 71 nt, Homo sapiens
cugaaagggacuuccaaggaagaugccaugaauaugugucauggcaucuuccuuggaagucccuuucaguu

SEQ ID NO: 48 - RNA, 71 nt, Homo sapiens
ugaaagggacuuccaaggaagaugccaugaaauauguguucauggcaucuuccuuggaagucccuuucauu

SEQ ID NO: 49 - RNA, 71 nt, Homo sapiens
gaaagggacuuccaaggaagaugccaugaaaauauguguuucauggcaucuuccuuggaagucccuuucuu

SEQ ID NO: 50 - RNA, 71 nt, Homo sapiens
aaagggacuuccaaggaagaugccaugaaagauaugugcuuucauggcaucuuccuuggaagucccuuuuu

SEQ ID NO: 51 - RNA, 71 nt, Homo sapiens
aagggacuuccaaggaagaugccaugaaagcauauguggcuuucauggcaucuuccuuggaagucccuuuu

SEQ ID NO: 52 - RNA, 71 nt, Homo sapiens
agggacuuccaaggaagaugccaugaaagcuauaugugagcuuucauggcaucuuccuuggaagucccuuu

SEQ ID NO: 53 - RNA, 71 nt, Homo sapiens
gggacuuccaaggaagaugccaugaaagcuuauaugugaagcuuucauggcaucuuccuuggaagucccuu

SEQ ID NO: 54 - RNA, 71 nt, Homo sapiens
ggacuuccaaggaagaugccaugaaagcuuaauauguguaagcuuucauggcaucuuccuuggaaguccuu

SEQ ID NO: 55 - RNA, 71 nt, Homo sapiens
gacuuccaaggaagaugccaugaaagcuuacauaugugguaagcuuucauggcaucuuccuuggaagucuu

SEQ ID NO: 56 - RNA, 71 nt, Homo sapiens
acuuccaaggaagaugccaugaaagcuuacaauauguguguaagcuuucauggcaucuuccuuggaaguuu

SEQ ID NO: 57 - RNA, 71 nt, Homo sapiens
cuuccaaggaagaugccaugaaagcuuacauauaugugauguaagcuuucauggcaucuuccuuggaaguu

SEQ ID NO: 58 - RNA, 71 nt, Homo sapiens
uuccaaggaagaugccaugaaagcuuacaucauauguggauguaagcuuucauggcaucuuccuuggaauu

SEQ ID NO: 59 - RNA, 71 nt, Homo sapiens
uccaaggaagaugccaugaaagcuuacaucaauaugugugauguaagcuuucauggcaucuuccuuggauu

SEQ ID NO: 60 - RNA, 71 nt, Homo sapiens
ccaaggaagaugccaugaaagcuuacaucaaauauguguugauguaagcuuucauggcaucuuccuugguu

SEQ ID NO: 61 - RNA, 71 nt, Homo sapiens
caaggaagaugccaugaaagcuuacaucaacauaugugguugauguaagcuuucauggcaucuuccuuguu

SEQ ID NO: 62 - RNA, 21 nt, Homo sapiens
gaugccuggaaugagcugauu

SEQ ID NO: 63 - RNA, 21 nt, Homo sapiens
augccuggaaugagcugaauu

SEQ ID NO: 64 - RNA, 21 nt, Homo sapiens
ugccuggaaugagcugaaauu

SEQ ID NO: 65 - RNA, 21 nt, Homo sapiens
gccuggaaugagcugaaaguu

SEQ ID NO: 66 - RNA, 21 nt, Homo sapiens
ccuggaaugagcugaaagguu

SEQ ID NO: 67 - RNA, 21 nt, Homo sapiens
cuggaaugagcugaaaggguu

SEQ ID NO: 68 - RNA, 21 nt, Homo sapiens
uggaaugagcugaaagggauu

SEQ ID NO: 69 - RNA, 21 nt, Homo sapiens
ggaaugagcugaaagggacuu

SEQ ID NO: 70 - RNA, 21 nt, Homo sapiens
gaaugagcugaaagggacuuu

SEQ ID NO: 71 - RNA, 21 nt, Homo sapiens
aaugagcugaaagggacuuuu

SEQ ID NO: 72 - RNA, 21 nt, Homo sapiens
augagcugaaagggacuucuu

SEQ ID NO: 73 - RNA, 21 nt, Homo sapiens
ugagcugaaagggacuuccuu

SEQ ID NO: 74 - RNA, 21 nt, Homo sapiens
gagcugaaagggacuuccauu

SEQ ID NO: 75 - RNA, 21 nt, Homo sapiens
agcugaaagggacuuccaauu

SEQ ID NO: 76 - RNA, 21 nt, Homo sapiens
gcugaaagggacuuccaaguu

SEQ ID NO: 77 - RNA, 21 nt, Homo sapiens
cugaaagggacuuccaagguu

SEQ ID NO: 78 - RNA, 21 nt, Homo sapiens
ugaaagggacuuccaaggauu

SEQ ID NO: 79 - RNA, 21 nt, Homo sapiens
gaaagggacuuccaaggaauu

SEQ ID NO: 80 - RNA, 21 nt, Homo sapiens
aaagggacuuccaaggaaguu

SEQ ID NO: 81 - RNA, 21 nt, Homo sapiens
aagggacuuccaaggaagauu

SEQ ID NO: 82 - RNA, 21 nt, Homo sapiens
agggacuuccaaggaagauuu

SEQ ID NO: 83 - RNA, 21 nt, Homo sapiens
gggacuuccaaggaagauguu

SEQ ID NO: 84 - RNA, 21 nt, Homo sapiens
ggacuuccaaggaagaugcuu

SEQ ID NO: 85 - RNA, 21 nt, Homo sapiens
gacuuccaaggaagaugccuu

SEQ ID NO: 86 - RNA, 21 nt, Homo sapiens
acuuccaaggaagaugccauu

SEQ ID NO: 87 - RNA, 21 nt, Homo sapiens
cuuccaaggaagaugccauuu

SEQ ID NO: 88 - RNA, 21 nt, Homo sapiens
uuccaaggaagaugccauguu

SEQ ID NO: 89 - RNA, 21 nt, Homo sapiens
uccaaggaagaugccaugauu

SEQ ID NO: 90 - RNA, 21 nt, Homo sapiens
ccaaggaagaugccaugaauu

SEQ ID NO: 91 - RNA, 21 nt, Homo sapiens
caaggaagaugccaugaaauu

SEQ ID NO: 92 - RNA, 21 nt, Homo sapiens
ucagcucauuccaggcaucuu

SEQ ID NO: 93 - RNA, 21 nt, Homo sapiens
uucagcucauuccaggcauuu

SEQ ID NO: 94 - RNA, 21 nt, Homo sapiens
uuucagcucauuccaggcauu

SEQ ID NO: 95 - RNA, 21 nt, Homo sapiens
cuuucagcucauuccaggcuu

SEQ ID NO: 96 - RNA, 21 nt, Homo sapiens
ccuuucagcucauuccagguu

SEQ ID NO: 97 - RNA, 21 nt, Homo sapiens
cccuuucagcucauuccaguu

SEQ ID NO: 98 - RNA, 21 nt, Homo sapiens
ucccuuucagcucauuccauu

SEQ ID NO: 99 - RNA, 21 nt, Homo sapiens
gucccuuucagcucauuccuu

SEQ ID NO: 100 - RNA, 21 nt, Homo sapiens
agucccuuucagcucauucuu

SEQ ID NO: 101 - RNA, 21 nt, Homo sapiens
aagucccuuucagcucauuuu

SEQ ID NO: 102 - RNA, 21 nt, Homo sapiens
gaagucccuuucagcucauuu

SEQ ID NO: 103 - RNA, 21 nt, Homo sapiens
ggaagucccuuucagcucauu

SEQ ID NO: 104 - RNA, 21 nt, Homo sapiens
uggaagucccuuucagcucuu

SEQ ID NO: 105 - RNA, 21 nt, Homo sapiens
uuggaagucccuuucagcuuu

SEQ ID NO: 106 - RNA, 21 nt, Homo sapiens
cuuggaagucccuuucagcuu

SEQ ID NO: 107 - RNA, 21 nt, Homo sapiens
ccuuggaagucccuuucaguu

SEQ ID NO: 108 - RNA, 21 nt, Homo sapiens
uccuuggaagucccuuucauu

SEQ ID NO: 109 - RNA, 21 nt, Homo sapiens
uuccuuggaagucccuuucuu

SEQ ID NO: 110 - RNA, 21 nt, Homo sapiens
cuuccuuggaagucccuuuuu

SEQ ID NO: 111 - RNA, 21 nt, Homo sapiens
ucuuccuuggaagucccuuuu

SEQ ID NO: 112 - RNA, 21 nt, Homo sapiens
aucuuccuuggaagucccuuu

SEQ ID NO: 113 - RNA, 21 nt, Homo sapiens
caucuuccuuggaagucccuu

SEQ ID NO: 114 - RNA, 21 nt, Homo sapiens
gcaucuuccuuggaaguccuu

SEQ ID NO: 115 - RNA, 21 nt, Homo sapiens
ggcaucuuccuuggaagucuu

SEQ ID NO: 116 - RNA, 21 nt, Homo sapiens
uggcaucuuccuuggaaguuu

SEQ ID NO: 117 - RNA, 21 nt, Homo sapiens
auggcaucuuccuuggaaguu

SEQ ID NO: 118 - RNA, 21 nt, Homo sapiens
cauggcaucuuccuuggaauu

SEQ ID NO: 119 - RNA, 21 nt, Homo sapiens
ucauggcaucuuccuuggauu

SEQ ID NO: 120 - RNA, 21 nt, Homo sapiens
uucauggcaucuuccuugguu

SEQ ID NO: 121 - RNA, 21 nt, Homo sapiens
uuucauggcaucuuccuuguu

SEQ ID NO: 122 - RNA, 31 nt, Homo sapiens
uuucagcucauuccaggcaucccacuuggcc

SEQ ID NO: 123 - RNA, 31 nt, Homo sapiens
cuuucagcucauuccaggcaucccacuuggc

SEQ ID NO: 124 - RNA, 31 nt, Homo sapiens
ccuuucagcucauuccaggcaucccacuugg

SEQ ID NO: 125 - RNA, 31 nt, Homo sapiens
cccuuucagcucauuccaggcaucccacuug

SEQ ID NO: 126 - RNA, 31 nt, Homo sapiens
ucccuuucagcucauuccaggcaucccacuu

SEQ ID NO: 127 - RNA, 31 nt, Homo sapiens
gucccuuucagcucauuccaggcaucccacu

SEQ ID NO: 128 - RNA, 31 nt, Homo sapiens
agucccuuucagcucauuccaggcaucccac

SEQ ID NO: 129 - RNA, 31 nt, Homo sapiens
aagucccuuucagcucauuccaggcauccca

SEQ ID NO: 130 - RNA, 31 nt, Homo sapiens
gaagucccuuucagcucauuccaggcauccc

SEQ ID NO: 131 - RNA, 31 nt, Homo sapiens
ggaagucccuuucagcucauuccaggcaucc

SEQ ID NO: 132 - RNA, 31 nt, Homo sapiens
uggaagucccuuucagcucauuccaggcauc

SEQ ID NO: 133 - RNA, 31 nt, Homo sapiens
uuggaagucccuuucagcucauuccaggcau

SEQ ID NO: 134 - RNA, 31 nt, Homo sapiens
cuuggaagucccuuucagcucauuccaggca

SEQ ID NO: 135 - RNA, 31 nt, Homo sapiens
ccuuggaagucccuuucagcucauuccaggc

SEQ ID NO: 136 - RNA, 31 nt, Homo sapiens
uccuuggaagucccuuucagcucauuccagg

SEQ ID NO: 137 - RNA, 31 nt, Homo sapiens
uuccuuggaagucccuuucagcucauuccag

SEQ ID NO: 138 - RNA, 31 nt, Homo sapiens
cuuccuuggaagucccuuucagcucauucca

SEQ ID NO: 139 - RNA, 31 nt, Homo sapiens
ucuuccuuggaagucccuuucagcucauucc

SEQ ID NO: 140 - RNA, 31 nt, Homo sapiens
aucuuccuuggaagucccuuucagcucauuc

SEQ ID NO: 141 - RNA, 31 nt, Homo sapiens
caucuuccuuggaagucccuuucagcucauu

SEQ ID NO: 142 - RNA, 31 nt, Homo sapiens
gcaucuuccuuggaagucccuuucagcucau

SEQ ID NO: 143 - RNA, 31 nt, Homo sapiens
ggcaucuuccuuggaagucccuuucagcuca

SEQ ID NO: 144 - RNA, 31 nt, Homo sapiens
uggcaucuuccuuggaagucccuuucagcuc

SEQ ID NO: 145 - RNA, 31 nt, Homo sapiens
auggcaucuuccuuggaagucccuuucagcu

SEQ ID NO: 146 - RNA, 31 nt, Homo sapiens
cauggcaucuuccuuggaagucccuuucagc

SEQ ID NO: 147 - RNA, 31 nt, Homo sapiens
ucauggcaucuuccuuggaagucccuuucag

SEQ ID NO: 148 - RNA, 31 nt, Homo sapiens
uucauggcaucuuccuuggaagucccuuuca

SEQ ID NO: 149 - RNA, 31 nt, Homo sapiens
uuucauggcaucuuccuuggaagucccuuuc

SEQ ID NO: 150 - RNA, 31 nt, Homo sapiens
cuuucauggcaucuuccuuggaagucccuuu

SEQ ID NO: 151 - RNA, 31 nt, Homo sapiens
gcuuucauggcaucuuccuuggaagucccuu

SEQ ID NO: 152 - RNA, 33 nt, Homo sapiens
ggccaagugggaugccuggaaugagcugaaauu

SEQ ID NO: 153 - RNA, 33 nt, Homo sapiens
gccaagugggaugccuggaaugagcugaaaguu

SEQ ID NO: 154 - RNA, 33 nt, Homo sapiens
ccaagugggaugccuggaaugagcugaaagguu

SEQ ID NO: 155 - RNA, 33 nt, Homo sapiens
caagugggaugccuggaaugagcugaaaggguu

SEQ ID NO: 156 - RNA, 33 nt, Homo sapiens
aagugggaugccuggaaugagcugaaagggauu

SEQ ID NO: 157 - RNA, 33 nt, Homo sapiens
agugggaugccuggaaugagcugaaagggacuu

SEQ ID NO: 158 - RNA, 33 nt, Homo sapiens
gugggaugccuggaaugagcugaaagggacuuu

SEQ ID NO: 159 - RNA, 33 nt, Homo sapiens
ugggaugccuggaaugagcugaaagggacuuuu

SEQ ID NO: 160 - RNA, 33 nt, Homo sapiens
gggaugccuggaaugagcugaaagggacuucuu

SEQ ID NO: 161 - RNA, 33 nt, Homo sapiens
ggaugccuggaaugagcugaaagggacuuccuu

SEQ ID NO: 162 - RNA, 33 nt, Homo sapiens
gaugccuggaaugagcugaaagggacuuccauu

SEQ ID NO: 163 - RNA, 33 nt, Homo sapiens
augccuggaaugagcugaaagggacuuccaauu

SEQ ID NO: 164 - RNA, 33 nt, Homo sapiens
ugccuggaaugagcugaaagggacuuccaaguu

SEQ ID NO: 165 - RNA, 33 nt, Homo sapiens
gccuggaaugagcugaaagggacuuccaagguu

SEQ ID NO: 166 - RNA, 33 nt, Homo sapiens
ccuggaaugagcugaaagggacuuccaaggauu

SEQ ID NO: 167 - RNA, 33 nt, Homo sapiens
cuggaaugagcugaaagggacuuccaaggaauu

SEQ ID NO: 168 - RNA, 33 nt, Homo sapiens
uggaaugagcugaaagggacuuccaaggaaguu

SEQ ID NO: 169 - RNA, 33 nt, Homo sapiens
ggaaugagcugaaagggacuuccaaggaagauu

SEQ ID NO: 170 - RNA, 33 nt, Homo sapiens
gaaugagcugaaagggacuuccaaggaagauuu

SEQ ID NO: 171 - RNA, 33 nt, Homo sapiens
aaugagcugaaagggacuuccaaggaagauguu

SEQ ID NO: 172 - RNA, 33 nt, Homo sapiens
augagcugaaagggacuuccaaggaagaugcuu

SEQ ID NO: 173 - RNA, 33 nt, Homo sapiens
ugagcugaaagggacuuccaaggaagaugccuu

SEQ ID NO: 174 - RNA, 33 nt, Homo sapiens
gagcugaaagggacuuccaaggaagaugccauu

SEQ ID NO: 175 - RNA, 33 nt, Homo sapiens
agcugaaagggacuuccaaggaagaugccauuu

SEQ ID NO: 176 - RNA, 33 nt, Homo sapiens
gcugaaagggacuuccaaggaagaugccauguu

SEQ ID NO: 177 - RNA, 33 nt, Homo sapiens
cugaaagggacuuccaaggaagaugccaugauu

SEQ ID NO: 178 - RNA, 33 nt, Homo sapiens
ugaaagggacuuccaaggaagaugccaugaauu

SEQ ID NO: 179 - RNA, 33 nt, Homo sapiens
gaaagggacuuccaaggaagaugccaugaaauu

SEQ ID NO: 180 - RNA, 33 nt, Homo sapiens
aaagggacuuccaaggaagaugccaugaaaguu

SEQ ID NO: 181 - RNA, 33 nt, Homo sapiens
aagggacuuccaaggaagaugccaugaaagcuu

SEQ ID NO: 182 - RNA, 21 nt, Homo sapiens
cggaaagacuguuccaaaauu

SEQ ID NO: 183 - RNA, 21 nt, Homo sapiens
uuuuggaacagucuuuccguu

SEQ ID NO: 184 - RNA, 21 nt, Homo sapiens
gagaaaggauuuggcuacauu

SEQ ID NO: 185 - RNA, 21 nt, Homo sapiens
uguagccaaauccuuucucuu

SEQ ID NO: 186 - RNA, 21 nt, Homo sapiens
caaaaacaguggauaauuuuu

SEQ ID NO: 187 - RNA, 21 nt, Homo sapiens
aaauuauccacuguuuuuguu

SEQ ID NO: 188 - RNA, 21 nt, Homo sapiens
gccaagaaccucaucaucuuu

SEQ ID NO: 189 - RNA, 21 nt, Homo sapiens
agaugaugagguucuuggcuu

SEQ ID NO: 190 - RNA, 21 nt, Homo sapiens
cggaaacgguccaggcuauuu

SEQ ID NO: 191 - RNA, 21 nt, Homo sapiens
auagccuggaccguuuccguu

SEQ ID NO: 192 - RNA, 21 nt, Homo sapiens
gaaccgcacugagcucauguu

SEQ ID NO: 193 - RNA, 21 nt, Homo sapiens
caugagcucagugcgguucuu

SEQ ID NO: 194 - RNA, 71 nt, Homo sapiens
guuuuuggaacagucuuuccgaagagaccaaauauguguuggucucuucggaaagacuguuccaaaaacuu

SEQ ID NO: 195 - RNA, 71 nt, Homo sapiens
cggaaagacuguuccaaaaacaguggauaauauaugugauuauccacuguuuuuggaacagucuuuccguu

SEQ ID NO: 196 - RNA, 71 nt, Homo sapiens
uuuguagccaaauccuuucucuccuguagcuauaugugagcuacaggagagaaaggauuuggcuacaaauu

SEQ ID NO: 197 - RNA, 71 nt, Homo sapiens
gagaaaggauuuggcuacaaaaacagcaaauauaugugauuugcuguuuuuguagccaaauccuuucucuu

SEQ ID NO: 198 - RNA, 71 nt, Homo sapiens
caaaauuauccacuguuuuuggaacagucuuauaugugaagacuguuccaaaaacaguggauaauuuuguu

SEQ ID NO: 199 - RNA, 71 nt, Homo sapiens
caaaaacaguggauaauuuuguggccuuagcauauguggcuaaggccacaaaauuauccacuguuuuuguu

SEQ ID NO: 200 - RNA, 71 nt, Homo sapiens
gaagaugaugagguucuuggcggcugucuguauaugugacagacagccgccaagaaccucaucaucuucuu

SEQ ID NO: 201 - RNA, 71 nt, Homo sapiens
gccaagaaccucaucaucuuccugggcgaugauaugugcaucgcccaggaagaugaugagguucuuggcuu

SEQ ID NO: 202 - RNA, 71 nt, Homo sapiens
acauagccuggaccguuuccguauaggaggaauauguguccuccuauacggaaacgguccaggcuauguuu

SEQ ID NO: 203 - RNA, 71 nt, Homo sapiens
cggaaacgguccaggcuaugugcucaaggacauaugugguccuugagcacauagccuggaccguuuccguu

SEQ ID NO: 204 - RNA, 71 nt, Homo sapiens
ugcaugagcucagugcgguuccacacauaccauauguggguauguguggaaccgcacugagcucaugcauu

SEQ ID NO: 205 - RNA, 71 nt, Homo sapiens
gaaccgcacugagcucaugcaggcuucccugauaugugcagggaagccugcaugagcucagugcgguucuu